(19) United States
(12) Patent Application Publication
(10) Pub. No.: US 2026/0315780 A1
(43) Pub. Date: Oct. 15, 2026

(54) FILTER SYSTEM FOR INTRACORPOREAL DEVICE

(71) Applicant: MAGENTA MEDICAL LTD, Kadima (IL)

(72) Inventors: Yosi TUVAL, Even Yehuda (IL); Gad LUBINSKY, Ein Vered (IL); Yuri SUDIN, Modiin-Makkabim-Reut (IL); Avi ROZENFELD, Haifa (IL); Shaul MUSTACCHI, Rosh Haayin (IL); Ran TAMIR, Ramat Gan (IL); Yuval ZIPORY, Modiin (IL); Rinat FRIEDMAN, Hadera (IL); Hadas KUSHNIR, Tel Aviv (IL); Tomer LARK, Herzliya (IL)

(21) Appl. No.: 19/463,581

(22) Filed: Jan. 29, 2026

Related U.S. Application Data
(63) Continuation of application No. PCT/IB2024/063109, filed on Dec. 23, 2024.
(60) Provisional application No. 63/615,377, filed on Dec. 28, 2023.
(60) Provisional application No. 63/566,681, filed on Mar. 18, 2024.
(60) Provisional application No. 63/692,734, filed on Sep. 10, 2024.

(51) Int. Cl. A61M 60/829; A61M 60/13; A61M 60/174; A61M 60/216; A61M 60/414; A61M 60/818; A61M 60/90

(52) U.S. Cl. CPC A61M 60/829; A61M 60/13; A61M 60/174; A61M 60/216; A61M 60/414; A61M 60/818; A61M 60/90; A61M 2205/3334; A61M 2205/7527; A61M 2209/06; A61M 2210/125; A61M 2210/127

(57) ABSTRACT

Apparatus and methods are described including connecting a proximal end of a purging-fluid tube to a purging-fluid bag, which contains purging fluid, via a proximal air-eliminating filter configured to remove air from the purging fluid, and connecting a distal end of the purging-fluid tube to an inlet port of an intracorporeal device via a distal air-eliminating filter configured to remove air from the purging fluid, such that the purging fluid flows from the purging-fluid bag, via the proximal air-eliminating filter, purging-fluid tube, and distal air-eliminating filter, into the inlet port. Other applications are also described.

### CROSS-REFERENCE TO RELATED APPLICATIONS

The present application is a continuation of PCT Application No. PCT/IB2024/063109 to Tuval et al. (published as WO 25/141457), entitled “Packaging for blood pumps,” filed Dec. 23, 2024, which claims priority from U.S. Provisional Application No. 63/615,377 to Tuval et al., entitled “Inlet guards for blood pumps,” filed Dec. 28, 2023, U.S. Provisional Application No. 63/566,681 to Tuval et al., entitled “Inlet guards for blood pumps,” filed Mar. 18, 2024, and U.S. Provisional Application No. 63/692,734 to Tuval et al., entitled “Inlet guards for blood pumps,” filed Sep. 10, 2024. All of the aforementioned applications are incorporated herein by reference.

### FIELD OF EMBODIMENTS

Some embodiments relate generally to medical devices, and specifically to blood pumps, e.g., for ventricular assist devices.

### BACKGROUND

Ventricular assist devices are mechanical circulatory support devices designed to assist and unload cardiac chambers in order to maintain or augment cardiac output. They are used in patients suffering from a failing heart and in patients at risk for deterioration of cardiac function during percutaneous coronary interventions. Most commonly, a left-ventricular assist device is applied to a defective heart in order to assist left-ventricular functioning. In some cases, a right-ventricular assist device is used in order to assist right-ventricular functioning. Such ventricular assist devices are either designed to be permanently implanted or mounted on a catheter for temporary placement.

### SUMMARY

Some embodiments of the present disclosure include packaging for an intracorporeal device, such as a ventricular assist device. The device includes a delivery catheter, a proximal element, which is disposed proximally to the delivery catheter and is wider than the delivery catheter, an elongate element passing through the proximal element and through the delivery catheter, and a self-expandable element coupled to the elongate element distally to the elongate element and configured for percutaneous delivery to a portion of a body of a subject while the self-expandable element is in a radially-constrained configuration within the delivery catheter. The packaging includes a tray shaped to define a chamber in which the self-expandable element is packageable in a non-radially-constrained configuration. The tray is configured to stabilize the proximal element while the self-expandable element is retracted into the delivery catheter via retraction of the elongate element. The packaging further includes a securement piece coupled to the tray adjacently to the chamber, and configured to secure the distal end of the delivery catheter while the self-expandable element is retracted into the delivery catheter.

Other embodiments include a cartridge for facilitating the setup of an intracorporeal device, the device including an inlet port, an outlet port, and a pressure-sensing port. The cartridge, which in some embodiments is packaged with the device in the aforementioned packaging, includes a first port, a second port, and a third port, and holds multiple tubes. The tubes include (a) a purging-fluid tube, configured to connect a purging-fluid bag, which contains purging fluid, to the inlet port, (b) a waste tube, configured to connect a waste bag to the outlet port, (c) a flushing tube, configured to connect a flushing-fluid bag, which contains flushing fluid, to a first sensor port of a pressure sensor, and (d) a pressure-sensing tube, configured to connect a second sensor port of the pressure sensor to the pressure-sensing port of the intracorporeal device such that the flushing fluid flows, via the pressure sensor, into the pressure-sensing port.

Typically, to facilitate properly connecting the tubes, the cartridge holds the tubes such that respective proximal portions of the purging-fluid tube, the waste tube, and the flushing tube pass through the first port, respective distal portions of the purging-fluid tube, the waste tube, and the pressure-sensing tube pass through the second port, and a distal portion of the flushing tube and a proximal portion of the pressure-sensing tube pass through the third port.

Alternatively or additionally, the cartridge is configured for insertion into a console such that the cartridge and the console interact with one another. For example, in some embodiments, the cartridge comprises pumps configured to pump the purging fluid through the purging-fluid tube and waste tube, and the console comprises motors configured to drive the pumps.

Other embodiments include a blood pump including an inlet guard. The inlet guard includes a main body, which is typically frustoconical and is shaped to define one or more blood-inlet openings configured to allow passage of blood therethrough. The inlet guard further includes multiple proximal flaps extending proximally from the main body. The blood pump further includes a frame including a proximal portion, a central portion, and a distal portion, an inner lining that lines at least part of the central portion of the frame, an impeller, which is configured to pump blood proximally, disposed within the frame, and a pump-outlet tube, which is fixed over the proximal portion of the frame and at least part of the central portion of the frame, and which is heat welded to the inner lining. The inlet guard is distal to the impeller, with the proximal flaps of the inlet guard being disposed between the pump-outlet tube and the inner lining where the pump-outlet tube and the inner lining are heat welded to one another. To facilitate coupling the inlet guard in this manner, the inlet guard is typically made of a material having a glass-transition temperature that is higher than respective glass-transition temperatures of each of the inner lining and the pump-outlet tube. Typically, the frame defines struts and the proximal flaps do not overlap any of the struts of the frame.

In some embodiments, to manufacture a frustoconical inlet guard, the blood-inlet openings are formed in a sheet of material, and the sheet of material is then rolled into the frustoconical shape. The inlet guard is coupled to the pump-outlet tube, e.g., via proximal flaps as described above, and is fixed over the distal portion of the frame.

Yet other embodiments include a left-ventricular assist device including an inflatable element, various embodiments of which are described herein. The device further includes a pump-outlet tube, which in some embodiments includes a lateral wall shaped to define one or more blood-outlet openings and is configured for insertion, through an aorta of a subject, into a left ventricle of a heart of the subject such that the blood-outlet openings are disposed within the aorta and a distal portion of the pump-outlet tube is disposed within the left ventricle. The device further includes an impeller configured to pump blood of the subject proximally through the pump-outlet tube, such that the blood exits the pump-outlet tube via the blood-outlet openings. The device further includes a delivery tube configured to extend, from outside the body of the subject, through the pump-outlet tube to the distal portion, and a drive cable passing through the delivery tube and configured to rotate the impeller. The inflatable element surrounds the delivery tube proximally to the blood-outlet openings. Advantageously, the inflatable element helps prevent injury to the aortic wall and/or centers the proximal portion of the pump-outlet tube in the aorta.

Yet other embodiments include a guide for inserting the distal end of a guidewire, which is typically soft and atraumatic, through a valve at the proximal end of an intracorporeal device, such as a ventricular assist device. The guide includes a tube, configured to radially constrain the guidewire, and a tube shell, which contains the tube. The tube shell is shaped to define a proximal shell opening, which is in communication with the proximal end of the tube, and includes a hollow distal shell portion, which contains the distal end of the tube and is configured for placement over the proximal end of the device until the distal end of the tube passes through the valve. The distal end of the guidewire is inserted into the proximal end of the intracorporeal device via the proximal shell opening and tube.

There is therefore provided, in accordance with some embodiments, an apparatus for use with a device that includes a delivery catheter, a proximal element, which is disposed proximally to the delivery catheter and is wider than the delivery catheter, an elongate element passing through the proximal element and through the delivery catheter, and a self-expandable element coupled to the elongate element distally to the elongate element and configured for percutaneous delivery to a portion of a body of a subject while the self-expandable element is in a radially-constrained configuration within the delivery catheter. The apparatus includes packaging. The packaging includes a tray shaped to define a chamber in which the self-expandable element is packageable in a non-radially-constrained configuration, and configured to stabilize the proximal element while the self-expandable element is retracted into the delivery catheter via retraction of the elongate element. The packaging further includes a securement piece coupled to the tray adjacently to the chamber, and configured to secure a distal end of the delivery catheter while the self-expandable element is retracted into the delivery catheter.

In some embodiments:
the delivery catheter of the device is coupled to an inlet port and the device additionally includes a purging-fluid bag containing purging fluid, and a purging-fluid tube, configured to connect the purging-fluid bag to the inlet port via an air-eliminating filter configured to remove air from the purging fluid; and
the tray includes a compartment configured to hold the air-eliminating filter in an upright position while the purging fluid flows from the purging-fluid bag, via the purging-fluid tube and air-eliminating filter, into the inlet port.

In some embodiments, the proximal element includes a fixation unit configured to fix a position of the elongate element relative to the delivery catheter.

In some embodiments, a portion of the tray underneath the securement piece slopes downwardly in a direction of the chamber.

In some embodiments,
the device further includes an inlet port,
the delivery catheter is coupled to the inlet port,
the device is for use with:
   a purging-fluid bag containing purging fluid, and
   a purging-fluid tube, configured to connect the purging-fluid bag to the inlet port via an air-eliminating filter configured to remove air from the purging fluid, and
the tray includes a compartment configured to hold the air-eliminating filter in an upright position while the purging fluid flows from the purging-fluid bag, via the purging-fluid tube and air-eliminating filter, into the inlet port.

In some embodiments, the tray is further shaped to define a track in which the delivery catheter is packageable.

In some embodiments, the packaging further includes a detachable element reversibly coupled to the tray over the track and configured to stabilize the proximal element when the proximal element is pushed against the detachable element.

In some embodiments, the track includes a widened portion configured to stabilize the proximal element when the proximal element is pushed against a wall of the widened portion.

There is further provided, in accordance with some embodiments, a method including removing a cover from a tray in which an intracorporeal device is packaged. The device includes a delivery catheter, a proximal element, which is disposed proximally to the delivery catheter and is wider than the delivery catheter, an elongate element passing through the proximal element and through the delivery catheter, and a self-expandable element coupled to the elongate element distally to the elongate element, packaged in a non-radially-constrained configuration, and configured for percutaneous delivery to a portion of a body of a subject while the self-expandable element is in a radially-constrained configuration within the delivery catheter. The method further includes stabilizing the proximal element using the tray, while retracting the elongate element so as to retract the self-expandable element into the delivery catheter.

In some embodiments, a detachable element is reversibly coupled to the tray over the track, and stabilizing the proximal element includes stabilizing the proximal element by pushing the proximal element against the detachable element.

In some embodiments, the track includes a widened portion, and stabilizing the proximal element includes stabilizing the proximal element by pushing the proximal element against a wall of the widened portion.

In some embodiments,
the device further includes a first component and a second component, and
the method further includes, prior to retracting the elongate element, purging an interface between the first component and the second component by passing a purging fluid through the device.

In some embodiments,
the device further includes an inflatable element, and
the method further includes inflating the inflatable element with the purging fluid by passing the purging fluid through the device.

In some embodiments, the method further includes, subsequently to passing the purging fluid through the device and prior to retracting the elongate element, deflating the inflatable element by suctioning the purging fluid from the device.

In some embodiments, the inflatable element surrounds the elongate element.

In some embodiments, a wall of the elongate element is shaped to define one or more inflation-fluid openings, the inflatable element surrounds the inflation-fluid openings, and inflating the inflatable element includes inflating the inflatable element via the inflation-fluid openings.

In some embodiments,
the self-expandable element includes:
   a pump-outlet tube, which is configured for insertion, through an aorta of the subject, into a left ventricle of a heart of the subject such that a proximal portion of the pump-outlet tube is disposed within the aorta and a distal portion of the pump-outlet tube is disposed within the left ventricle, and
   an impeller configured to pump blood of the subject proximally through the pump-outlet tube, such that the blood exits the proximal portion of the pump-outlet tube, the first component of the intracorporeal device includes an axial shaft coupled to the impeller and configured to rotate such that the impeller pumps the blood, and
the second component of the intracorporeal device includes at least one bearing configured not to rotate with the axial shaft.

In some embodiments,
the device defines a pressure-sensing channel, and
the method further includes, prior to retracting the elongate element, flushing the pressure-sensing channel, with a flushing fluid, at a flow rate higher than a usual flow rate at which the flushing fluid flows through the pressure-sensing channel while the device is in use within the body of the subject.

In some embodiments, the pressure-sensing channel is between the delivery catheter and the elongate element.

In some embodiments,
the tray is shaped to define a chamber,
the self-expandable element is packaged in the chamber, and
the method further includes, prior to flushing the pressure-sensing channel, at least partly filling the chamber with a liquid.

There is further provided, in accordance with some embodiments, an apparatus for use with a device. The device includes a delivery catheter coupled to an inlet port, an elongate element passing through the delivery catheter, and a self-expandable element coupled to the elongate element distally to the elongate element and configured for percutaneous delivery to a portion of a body of a subject while the self-expandable element is in a radially-constrained configuration within the delivery catheter, a purging-fluid bag containing purging fluid, and a purging-fluid tube, configured to connect the purging-fluid bag to the inlet port via an air-eliminating filter configured to remove air from the purging fluid. The apparatus includes packaging. The packaging includes a tray shaped to define a chamber in which the self-expandable element is packageable in a non-radially-constrained configuration, and including a compartment configured to hold the air-eliminating filter in an upright position while the purging fluid flows from the purging-fluid bag, via the purging-fluid tube and air-eliminating filter, into the inlet port.

In some embodiments, the packaging further includes a securement piece coupled to the tray adjacently to the chamber, and configured to secure a distal end of the delivery catheter while the self-expandable element is retracted into the delivery catheter via retraction of the elongate element.

In some embodiments, a portion of the tray underneath the securement piece slopes downwardly in a direction of the chamber.

In some embodiments, the tray is further shaped to define a track in which the delivery catheter is packageable.

In some embodiments,
the device further includes a proximal element, which is disposed proximally to the delivery catheter and is wider than the delivery catheter,
the elongate element passes through the proximal element, and
the tray is configured to stabilize the proximal element while the self-expandable element is retracted into the delivery catheter.

In some embodiments, the proximal element includes a fixation unit configured to fix a position of the elongate element relative to the delivery catheter.

In some embodiments,
the tray is further shaped to define a track in which the delivery catheter is packageable, and
the packaging includes a detachable element reversibly coupled to the tray over the track and configured to stabilize the proximal element when the proximal element is pushed against the detachable element.

In some embodiments,
the tray is further shaped to define a track in which the delivery catheter is packageable, and
the track includes a widened portion configured to stabilize the proximal element when the proximal element is pushed against a wall of the widened portion.

There is further provided, in accordance with some embodiments, a method including removing a cover from a tray in which an intracorporeal device is packaged, the tray including a compartment configured to hold an air-eliminating filter, which is configured to remove air from purging fluid, in an upright position. The device includes a delivery catheter coupled to an inlet port, an elongate element passing through the delivery catheter, and a self-expandable element coupled to the elongate element distally to the elongate element, packaged in a non-radially-constrained configuration, and configured for percutaneous delivery to a portion of a body of a subject while the self-expandable element is in a radially-constrained configuration within the delivery catheter. The method includes, using a purging-fluid tube, connecting a purging-fluid bag, which contains the purging fluid, to the inlet port via the air-eliminating filter while the air-eliminating filter is held within the compartment, such that the purging fluid flows from the purging-fluid bag, via the purging-fluid tube and air-eliminating filter, into the inlet port. The method further includes, subsequently to connecting the purging-fluid bag to the inlet port, retracting the self-expandable element into the delivery catheter by retracting the elongate element.

In some embodiments,
the device further includes a first component and a second component, and
the purging fluid purges an interface between the first component and the second component by flowing through the device via the inlet port.

In some embodiments,
the device further includes an inflatable element, and
the purging fluid inflates the inflatable element by flowing through the device via the inlet port.

In some embodiments, the method further includes, prior to retracting the elongate element, deflating the inflatable element by suctioning the purging fluid from the device.

In some embodiments, the inflatable element surrounds the elongate element.

In some embodiments, a wall of the elongate element is shaped to define one or more inflation-fluid openings, the inflatable element surrounds the inflation-fluid openings, and the purging fluid inflates the inflatable element via the inflation-fluid openings.

In some embodiments,
the self-expandable element includes:
   a pump-outlet tube, which is configured for insertion, through an aorta of the subject, into a left ventricle of a heart of the subject such that a proximal portion of the pump-outlet tube is disposed within the aorta and a distal portion of the pump-outlet tube is disposed within the left ventricle, and
   an impeller configured to pump blood of the subject proximally through the pump-outlet tube, such that the blood exits the proximal portion of the pump-outlet tube,
the first component of the intracorporeal device includes an axial shaft coupled to the impeller and configured to rotate such that the impeller pumps the blood, and
the second component of the intracorporeal device includes at least one bearing configured not to rotate with the axial shaft.

In some embodiments,
the device defines a pressure-sensing channel, and
the method further includes, prior to retracting the elongate element, flushing the pressure-sensing channel, with a flushing fluid, at a flow rate higher than a usual flow rate at which the flushing fluid flows through the pressure-sensing channel while the device is in use within the body of the subject.

In some embodiments, the pressure-sensing channel is between the delivery catheter and the elongate element.

In some embodiments,
the tray is shaped to define a chamber,
the self-expandable element is packaged in the chamber, and
the method further includes, prior to flushing the pressure-sensing channel, at least partly filling the chamber with a liquid.

There is further provided, in accordance with some embodiments, a method including, by passing a purging fluid through an intracorporeal device, which includes a first component, a second component, an inflatable element, and a self-expandable element, inflating the inflatable element with the purging fluid, and purging an interface between the first component and the second component. The method further includes, by suctioning the purging fluid from the intracorporeal device, deflating the inflatable element. The method further includes, subsequently to deflating the inflatable element, crimping the self-expandable element.

In some embodiments, passing the purging fluid through the intracorporeal device, deflating the inflatable element, and crimping the self-expandable element includes passing the purging fluid through the intracorporeal device, deflating the inflatable element, and crimping the self-expandable element prior to inserting the intracorporeal device into a body of a subject.

In some embodiments,
the device defines a pressure-sensing channel, and
the method further includes, prior to crimping the self-expandable element, flushing the pressure-sensing channel, with a flushing fluid, at a flow rate higher than a usual flow rate at which the flushing fluid flows through the pressure-sensing channel while the device is in use within the body of the subject.

In some embodiments, the method further includes, prior to flushing the pressure-sensing channel:
removing a cover from a tray in which the intracorporeal device is packaged, the tray being shaped to define a chamber in which the self-expandable element is packaged in a non-radially-constrained configuration; and
at least partly filling the chamber with a liquid.

In some embodiments,
the device further includes:
   a delivery catheter coupled to an inlet port, and
   an elongate element passing through the delivery catheter,
the self-expandable element is coupled to the elongate element distally to the elongate element,
crimping the self-expandable element includes crimping the self-expandable element by retracting the elongate element, thereby retracting the self-expandable element into the delivery catheter,
the method further includes removing a cover from a tray in which the intracorporeal device is packaged, the tray including a compartment configured to hold an air-eliminating filter, which is configured to remove air from the purging fluid, in an upright position, and
passing the purging fluid through the intracorporeal device includes passing the purging fluid through the intracorporeal device by, using a purging-fluid tube, connecting a purging-fluid bag, which contains the purging fluid, to the inlet port via the air-eliminating filter while the air-eliminating filter is held within the compartment, such that the purging fluid flows from the purging-fluid bag, via the purging-fluid tube and air-eliminating filter, into the inlet port.

In some embodiments,
the air-eliminating filter is a distal air-eliminating filter, and
connecting the purging-fluid bag to the inlet port includes:
   connecting the purging-fluid tube to the inlet port via the distal air-eliminating filter; and
   connecting the purging-fluid tube to the purging-fluid bag via a proximal air-eliminating filter, which is configured to remove air from the purging fluid.

In some embodiments, the proximal air-eliminating filter includes a proximal air-filtering membrane shaped to define proximal pores, and the distal air-eliminating filter includes a distal air-filtering membrane shaped to define distal pores, which are smaller than the proximal pores.

In some embodiments, the method further includes, prior to passing the purging fluid through the intracorporeal device, removing a cover from a tray in which the intracorporeal device is packaged, the tray being shaped to define a chamber in which the self-expandable element is packaged in a non-radially-constrained configuration,
the device further includes:
   a delivery catheter,
   a proximal element, which is disposed proximally to the delivery catheter and is wider than the delivery catheter, and
   an elongate element passing through the proximal element and through the delivery catheter, and
crimping the self-expandable element includes crimping the self-expandable element by retracting the elongate element so as to retract the self-expandable element into the delivery catheter, while stabilizing the proximal element using the tray.

In some embodiments, the proximal element includes a fixation unit configured to fix a position of the elongate element relative to the delivery catheter.

In some embodiments, the tray is shaped to define a track in which the delivery catheter is packaged.

In some embodiments, a detachable element is reversibly coupled to the tray over the track, and stabilizing the proximal element includes stabilizing the proximal element by pushing the proximal element against the detachable element.

In some embodiments, the track includes a widened portion, and stabilizing the proximal element includes stabilizing the proximal element by pushing the proximal element against a wall of the widened portion.

In some embodiments, passing the fluid through the intracorporeal device, deflating the inflatable element, and crimping the self-expandable element includes passing the fluid through the intracorporeal device, deflating the inflatable element, and crimping the self-expandable element after inserting the intracorporeal device into a body of a subject.

In some embodiments,
the self-expandable element includes:
   a pump-outlet tube, which is configured for insertion, through an aorta of a subject, into a left ventricle of a heart of the subject such that a proximal portion of the pump-outlet tube is disposed within the aorta and a distal portion of the pump-outlet tube is disposed within the left ventricle, and
   an impeller configured to pump blood of the subject proximally through the pump-outlet tube, such that the blood exits the proximal portion of the pump-outlet tube, the intracorporeal device further includes:
   a delivery tube configured to extend, from outside a body of the subject, through the pump-outlet tube to the distal portion, and
   a drive cable passing through the delivery tube and configured to rotate the impeller, and
the inflatable element surrounds the delivery tube.

In some embodiments, a wall of the delivery tube is shaped to define one or more inflation-fluid openings, the inflatable element surrounds the inflation-fluid openings, and inflating the inflatable element includes inflating the inflatable element via the inflation-fluid openings.

In some embodiments,
the first component of the intracorporeal device includes an axial shaft coupled to the impeller and configured to rotate such that the impeller pumps the blood, and
the second component of the intracorporeal device includes at least one bearing configured not to rotate with the axial shaft.

There is further provided, in accordance with some embodiments, an apparatus for use with a purging-fluid bag containing purging fluid and an intracorporeal device including an inlet port. The apparatus includes a proximal air-eliminating filter, configured to remove air from the purging fluid, and a distal air-eliminating filter, configured to remove air from the purging fluid. The apparatus further includes a purging-fluid tube, configured to connect to the purging-fluid bag via the proximal air-eliminating filter, and to connect to the inlet port via the distal air-eliminating filter, such that the purging fluid flows from the purging-fluid bag, via the proximal air-eliminating filter, purging-fluid tube, and distal air-eliminating filter, into the inlet port.

In some embodiments, the proximal air-eliminating filter includes a proximal air-filtering membrane shaped to define proximal pores, and the distal air-eliminating filter includes a distal air-filtering membrane shaped to define distal pores, which are smaller than the proximal pores.

In some embodiments,
the intracorporeal device further includes a self-expandable element,
the apparatus further includes packaging including a tray shaped to define a chamber in which the self-expandable element is packageable in a non-radially-constrained configuration, and
the tray includes a compartment configured to hold the distal air-eliminating filter in an upright position while the purging fluid flows from the purging-fluid bag, via the purging-fluid tube and distal air-eliminating filter, into the inlet port.

In some embodiments,
the intracorporeal device further includes a delivery catheter coupled to the inlet port and an elongate element passing through the delivery catheter,
the self-expandable element is coupled to the elongate element distally to the elongate element and is configured for percutaneous delivery to a portion of a body of a subject while the self-expandable element is in a radially-constrained configuration within the delivery catheter, and
the packaging further includes a securement piece coupled to the tray adjacently to the chamber, and configured to secure a distal end of the delivery catheter while the self-expandable element is retracted into the delivery catheter via retraction of the elongate element.

In some embodiments, a portion of the tray underneath the securement piece slopes downwardly in a direction of the chamber.

In some embodiments, the tray is further shaped to define a track in which the delivery catheter is packageable.

In some embodiments,
the device further includes a proximal element, which is disposed proximally to the delivery catheter and is wider than the delivery catheter,
the elongate element passes through the proximal element, and
the tray is configured to stabilize the proximal element while the self-expandable element is retracted into the delivery catheter.

In some embodiments, the proximal element includes a fixation unit configured to fix a position of the elongate element relative to the delivery catheter.

In some embodiments,
the tray is further shaped to define a track in which the delivery catheter is packageable, and
the packaging further includes a detachable element reversibly coupled to the tray over the track and configured to stabilize the proximal element when the proximal element is pushed against the detachable element.

In some embodiments,
the tray is further shaped to define a track in which the delivery catheter is packageable, and
the track includes a widened portion configured to stabilize the proximal element when the proximal element is pushed against a wall of the widened portion.

There is further provided, in accordance with some embodiments, a method including connecting a proximal end of a purging-fluid tube to a purging-fluid bag, which contains purging fluid, via a proximal air-eliminating filter configured to remove air from the purging fluid. The method further includes connecting a distal end of the purging-fluid tube to an inlet port of an intracorporeal device via a distal air-eliminating filter configured to remove air from the purging fluid, such that the purging fluid flows from the purging-fluid bag, via the proximal air-eliminating filter, purging-fluid tube, and distal air-eliminating filter, into the inlet port.

In some embodiments,
the device includes:
   a delivery catheter coupled to the inlet port,
   an elongate element passing through the delivery catheter, and
   a self-expandable element coupled to the elongate element distally to the elongate element and configured for percutaneous delivery to a portion of a body of a subject while the self-expandable element is in a radially-constrained configuration within the delivery catheter, and
the method further includes, subsequently to connecting the purging-fluid tube to the purging-fluid bag and to the inlet port, retracting the self-expandable element into the delivery catheter by retracting the elongate element.

In some embodiments,
the device further includes a first component and a second component, and
the purging fluid purges an interface between the first component and the second component by flowing through the device via the inlet port.

In some embodiments,
the device further includes an inflatable element, and
the purging fluid inflates the inflatable element by flowing through the device via the inlet port.

In some embodiments, the method further includes, prior to retracting the elongate element, deflating the inflatable element by suctioning the purging fluid from the device.

In some embodiments, the inflatable element surrounds the elongate element.

In some embodiments, a wall of the elongate element is shaped to define one or more inflation-fluid openings, the inflatable element surrounds the inflation-fluid openings, and the purging fluid inflates the inflatable element via the inflation-fluid openings.

In some embodiments,
the self-expandable element includes:
   a pump-outlet tube, which is configured for insertion, through an aorta of the subject, into a left ventricle of a heart of the subject such that a proximal portion of the pump-outlet tube is disposed within the aorta and a distal portion of the pump-outlet tube is disposed within the left ventricle, and
   an impeller configured to pump blood of the subject proximally through the pump-outlet tube, such that the blood exits the proximal portion of the pump-outlet tube,
the first component of the intracorporeal device includes an axial shaft coupled to the impeller and configured to rotate such that the impeller pumps the blood, and
the second component of the intracorporeal device includes at least one bearing configured not to rotate with the axial shaft.

In some embodiments,
the device defines a pressure-sensing channel, and
the method further includes, prior to retracting the elongate element, flushing the pressure-sensing channel, with a flushing fluid, at a flow rate higher than a usual flow rate at which the flushing fluid flows through the pressure-sensing channel while the device is in use within the body of the subject.

In some embodiments, the pressure-sensing channel is between the delivery catheter and the elongate element.

In some embodiments,
the device is packaged in a tray shaped to define a chamber, the self-expandable element being packaged in the chamber, and
the method further includes, prior to flushing the pressure-sensing channel, at least partly filling the chamber with a liquid.

There is further provided, in accordance with some embodiments, a method including inserting an apparatus, over a guidewire, into a left ventricle of a subject. The apparatus includes a pump-outlet tube, an impeller configured to pump blood of the subject proximally through the pump-outlet tube, a delivery tube that extends, from outside a body of the subject, through the pump-outlet tube, a drive cable passing through the delivery tube and configured to rotate the impeller, a distal-tip element that is distal to the pump-outlet tube, and a delivery catheter, which holds the pump-outlet tube in a radially-constrained configuration, the guidewire passing through the distal-tip element. The method further includes removing the pump-outlet tube from the delivery catheter within the left ventricle, such that the pump-outlet tube adopts a non-radially-constrained configuration. The method further includes, subsequently to removing the pump-outlet tube from the delivery catheter, positioning the distal-tip element at an apex of the left ventricle, and withdrawing the guidewire from the distal-tip element.

In some embodiments, positioning the distal-tip element at the apex includes positioning the distal-tip element at the apex by pushing the distal-tip element toward the apex while withdrawing the guidewire from the distal-tip element.

In some embodiments, the method further includes reinserting the pump-outlet tube into the delivery catheter within a descending aorta of the subject.

There is further provided, in accordance with some embodiments, a method including inserting an apparatus, over a guidewire, into a left ventricle of a subject. The apparatus includes a pump-outlet tube, an impeller configured to pump blood of the subject proximally through the pump-outlet tube, a delivery tube that extends, from outside a body of the subject, through the pump-outlet tube, a drive cable passing through the delivery tube and configured to rotate the impeller, and a distal-tip element that is distal to the pump-outlet tube, the guidewire passing through the distal-tip element. The method further includes positioning the distal-tip element at an apex of the left ventricle, by pushing the distal-tip element toward the apex while withdrawing the guidewire from the distal-tip element.

In some embodiments,
the apparatus further includes a delivery catheter, which holds the pump-outlet tube in a radially-constrained configuration, and
the method further includes removing the pump-outlet tube from the delivery catheter within the left ventricle, such that the pump-outlet tube adopts a non-radially-constrained configuration, before positioning the distal-tip element at the apex of the left ventricle.

In some embodiments, the method further includes reinserting the pump-outlet tube into the delivery catheter within a descending aorta of the subject.

There is further provided, in accordance with some embodiments, an apparatus for use with a guidewire and an intracorporeal device including a valve at a proximal end of the device. The apparatus includes a tube, configured to radially constrain the guidewire, and a tube shell, which contains the tube, is shaped to define a proximal shell opening, which is in communication with a proximal end of the tube, and includes a hollow distal shell portion, which contains a distal end of the tube and is configured for placement over the proximal end of the device until the distal end of the tube passes through the valve, such that a distal end of the guidewire is insertable into the proximal end of the intracorporeal device via the proximal shell opening and tube.

In some embodiments, an inner diameter of the tube is between 0.4 and 0.8 mm.

In some embodiments, the inner diameter of the tube is between 0.5 and 0.7 mm.

In some embodiments, the proximal shell opening is conical.

In some embodiments, the distal end of the guidewire is soft and atraumatic.

There is further provided, in accordance with some embodiments, a method for facilitating repositioning an intracorporeal device within a body of a subject. The method includes partially withdrawing an intracorporeal device, which includes a valve at a proximal end of the device, from the body of the subject. The method further includes placing a hollow distal shell portion of a tube shell, which contains a tube configured to radially constrain a guidewire and is shaped to define a proximal shell opening in communication with a proximal end of the tube, over the proximal end of the device until a distal end of the tube, which is disposed within the hollow distal shell portion, passes through the valve. The method further includes inserting a distal end of the guidewire into the proximal end of the intracorporeal device via the proximal shell opening and tube.

In some embodiments, the method further includes, prior to placing the hollow distal shell portion of the tube shell over the proximal end of the device, decoupling the proximal end of the device from a motor unit.

There is further provided, in accordance with some embodiments, an apparatus for use with a purging-fluid bag containing purging fluid, an intracorporeal device including an inlet port, an outlet port, and a pressure-sensing port, a waste bag, a flushing-fluid bag containing flushing fluid, and a pressure sensor including a first sensor port and a second sensor port. The apparatus includes multiple tubes, including a purging-fluid tube, configured to connect the purging-fluid bag to the inlet port of the intracorporeal device, a waste tube, configured to connect the waste bag to the outlet port of the intracorporeal device, a flushing tube, configured to connect the flushing-fluid bag to the first sensor port of the pressure sensor, and a pressure-sensing tube, configured to connect the second sensor port of the pressure sensor to the pressure-sensing port of the intracorporeal device such that the flushing fluid flows, via the pressure sensor, into the pressure-sensing port. The apparatus further includes a cartridge, which includes a first port, a second port, and a third port, and which holds the tubes such that respective proximal portions of the purging-fluid tube, the waste tube, and the flushing tube pass through the first port, respective distal portions of the purging-fluid tube, the waste tube, and the pressure-sensing tube pass through the second port, and a distal portion of the flushing tube and a proximal portion of the pressure-sensing tube pass through the third port.

In some embodiments, the cartridge further includes one or more pumps configured to pump the purging fluid from the purging-fluid bag, through the device, and into the waste bag.

In some embodiments, the pumps include:
respective barrels, which are connected to the purging-fluid tube and/or the waste tube; and
respective plungers or pistons configured to reciprocate within the barrels so as to pump the purging fluid.

In some embodiments, the pumps include respective rotors configured to squeeze the purging-fluid tube and/or the waste tube so as to pump the purging fluid.

In some embodiments,
the cartridge further includes an electrical interface configured to receive electrical power, and
the apparatus further includes an electric cable, which is connected to the electrical interface, exits the cartridge via the third port, and is configured to connect to the pressure sensor so as to deliver the electrical power to the pressure sensor.

In some embodiments, the apparatus further includes a console, and the cartridge is configured for insertion into the console.

In some embodiments,
the cartridge further includes one or more pumps configured to pump the purging fluid from the purging-fluid bag, through the device, and into the waste bag, and
the console includes one or more motors configured to drive the pumps following the insertion of the cartridge.

In some embodiments, the console includes one or more pumps configured to pump the purging fluid from the purging-fluid bag, through the device, and into the waste bag following the insertion of the cartridge.

In some embodiments,
the pumps include respective rotors,
the cartridge is shaped to define one or more openings, and
the cartridge is configured for insertion into the console such that during the insertion, the rotors pass through the openings, respectively, such that the rotors are positioned to squeeze the purging-fluid tube and/or the waste tube so as to pump the purging fluid.

In some embodiments, the cartridge further includes an electrical interface configured to receive electrical power from the console following the insertion of the cartridge.

In some embodiments,
the pressure sensor is a first pressure sensor, and
the console includes a second pressure sensor configured to sense a pressure in the purging-fluid tube following the insertion of the cartridge.

In some embodiments, the purging-fluid tube includes an expandable portion within the cartridge, and the pressure sensor is configured to sense the pressure by sensing an expansion of the expandable portion.

In some embodiments, the console further includes a latch configured to close on the cartridge upon the insertion of the cartridge, such that the expansion of the expandable portion does not cause the cartridge to exit the console.

In some embodiments, the console includes a cable interface, and the apparatus further includes a cable, configured to connect the console to the intracorporeal device via the cable interface.

In some embodiments, the apparatus further includes a cart configured to carry the console and shaped to define:
a first groove, configured to hold the cable,
a second groove, configured to hold the respective distal portions of the purging-fluid tube, the waste tube, and the pressure-sensing tube, and
a third groove, configured to hold the distal portion of the flushing tube and the proximal portion of the pressure-sensing tube.

In some embodiments, the cart is configured to carry the console such that, following the insertion of the cartridge:
the first groove is aligned with the cable interface,
the second groove is aligned with the second port, and
the third groove is aligned with the third port.

In some embodiments,
the console includes:
   a chassis, configured to connect to the intracorporeal device while the intracorporeal device is within a body of a subject;
   a processor disposed within the chassis and configured to control the intracorporeal device via the connection; and
   a console hook coupled to the chassis, and
the apparatus further includes a cart configured to carry the console,
   the console being removable from the cart and hangable, via the console hook, from a bedrail of a bed of the subject.

In some embodiments, the console hook is rotatably coupled to the chassis such that the console hook is rotatable from a closed position, in which the console hook does not protrude from the chassis, to an open position, in which the console hook protrudes from the chassis.

In some embodiments,
the chassis includes a ratchet and a release mechanism configured to release the ratchet, and
the console hook is coupled to the chassis via the ratchet such that:
   an activation of the release mechanism causes the console hook to rotate from the closed position to the open position, and
   following a rotation of the console hook from the open position to a partially-closed position in which the console hook secures the console on the bedrail, the ratchet maintains
the console hook in the partially-closed position.

In some embodiments, the release mechanism includes a handle coupled to the ratchet, and the activation of the release mechanism includes a lifting of the handle such that, when a user lifts the handle so as to carry the console, via the handle, from the cart to the bedrail, the console hook rotates to the open position.

In some embodiments, the chassis is shaped to define one or more indentations, and the cart includes one or more protrusions configured to fit into the indentations while the cart carries the console.

In some embodiments, the protrusions are at least partly magnetic, and the chassis further includes respective ferromagnetic elements adjacent to the indentations.

In some embodiments, the protrusions are at least partly ferromagnetic, and the chassis further includes respective magnetic elements adjacent to the indentations.

In some embodiments,
the cart includes a tray, which is configured to carry the console, and
the protrusions protrude upward from the tray.

In some embodiments,
the cart further includes a backstop, which is behind the tray,
the tray slants downward toward the backstop, and
the protrusions are spaced from the backstop so as to facilitate a backward tilt of the console while the console is on the tray.

There is further provided, in accordance with some embodiments, an apparatus for use with a purging-fluid bag containing purging fluid, an intracorporeal device including an inlet port, an outlet port, and a pressure-sensing port, a waste bag, a flushing-fluid bag containing flushing fluid, and a pressure sensor including a first sensor port and a second sensor port. The apparatus includes multiple tubes, including a purging-fluid tube, configured to connect the purging-fluid bag to the inlet port of the intracorporeal device, a waste tube, configured to connect the waste bag to the outlet port of the intracorporeal device, a flushing tube, configured to connect the flushing-fluid bag to the first sensor port of the pressure sensor, and a pressure-sensing tube, configured to connect the second sensor port of the pressure sensor to the pressure-sensing port of the intracorporeal device such that the flushing fluid flows, via the pressure sensor, into the pressure-sensing port. The apparatus further includes a console and a cartridge, which holds the tubes and is configured for insertion into the console such that the cartridge and the console interact with one another.

In some embodiments,
the cartridge includes one or more pumps configured to pump the purging fluid from the purging-fluid bag, through the device, and into the waste bag, and
the console includes one or more motors configured to drive the pumps following the insertion of the cartridge.

In some embodiments, the pumps include:
respective barrels, which are connected to the purging-fluid tube and/or the waste tube; and
respective plungers or pistons configured to reciprocate within the barrels, when driven by the motors, so as to pump the purging fluid.

In some embodiments, the pumps include respective rotors configured to squeeze the purging-fluid tube and/or the waste tube, when driven by the motors, so as to pump the purging fluid.

In some embodiments, the console includes one or more pumps configured to pump the purging fluid from the purging-fluid bag, through the device, and into the waste bag following the insertion of the cartridge.

In some embodiments,
the pumps include respective rotors,
the cartridge is shaped to define one or more openings, and
the cartridge is configured for insertion into the console such that during the insertion, the rotors pass through the openings, respectively, such that the rotors are positioned to squeeze the purging-fluid tube and/or the waste tube so as to pump the purging fluid.

In some embodiments, the cartridge includes an electrical interface configured to receive electrical power from the console following the insertion of the cartridge.

In some embodiments,
the pressure sensor is a first pressure sensor, and
the console includes a second pressure sensor configured to sense a pressure in the purging-fluid tube following the insertion of the cartridge.

In some embodiments, the purging-fluid tube includes an expandable portion within the cartridge, and the pressure sensor is configured to sense the pressure by sensing an expansion of the expandable portion.

In some embodiments, the console further includes a latch configured to close on the cartridge upon the insertion of the cartridge, such that the expansion of the expandable portion does not cause the cartridge to exit the console.

In some embodiments, the cartridge includes a first port, a second port, and a third port, and the cartridge holds the tubes such that:
respective proximal portions of the purging-fluid tube, the waste tube, and the flushing tube pass through the first port,
respective distal portions of the purging-fluid tube, the waste tube, and the pressure-sensing tube pass through the second port, and
a distal portion of the flushing tube and a proximal portion of the pressure-sensing tube pass through the third port.

In some embodiments,
the cartridge further includes an electrical interface configured to receive electrical power, and
the apparatus further includes an electric cable, which is connected to the electrical interface, exits the cartridge via the third port, and is configured to connect to the pressure sensor so as to deliver the electrical power to the pressure sensor.

In some embodiments, the console includes a cable interface, and the apparatus further includes a cable, configured to connect the console to the intracorporeal device via the cable interface.

In some embodiments, the apparatus further includes a cart configured to carry the console and shaped to define:
a first groove, configured to hold the cable,
a second groove, configured to hold respective distal portions of the purging-fluid tube, the waste tube, and the pressure-sensing tube, and
a third groove, configured to hold a distal portion of the flushing tube and a proximal portion of the pressure-sensing tube.

In some embodiments,
the console includes:
   a chassis, configured to connect to the intracorporeal device while the intracorporeal device is within a body of a subject;
   a processor disposed within the chassis and configured to control the intracorporeal device via the connection; and
   a console hook coupled to the chassis, and the apparatus further includes a cart configured to carry the console,
   the console being removable from the cart and hangable, via the console hook, from a bedrail of a bed of the subject.

In some embodiments, the console hook is rotatably coupled to the chassis such that the console hook is rotatable from a closed position, in which the console hook does not protrude from the chassis, to an open position, in which the console hook protrudes from the chassis.

In some embodiments,
the chassis includes a ratchet and a release mechanism configured to release the ratchet, and
the console hook is coupled to the chassis via the ratchet such that:
   an activation of the release mechanism causes the console hook to rotate from the closed position to the open position, and
   following a rotation of the console hook from the open position to a partially-closed position in which the console hook secures the console on the bedrail, the ratchet maintains the console hook in the partially-closed position.

In some embodiments, the release mechanism includes a handle coupled to the ratchet, and the activation of the release mechanism includes a lifting of the handle such that, when a user lifts the handle so as to carry the console, via the handle, from the cart to the bedrail, the console hook rotates to the open position.

In some embodiments, the chassis is shaped to define one or more indentations, and the cart includes one or more protrusions configured to fit into the indentations while the cart carries the console.

In some embodiments, the protrusions are at least partly magnetic, and the chassis further includes respective ferromagnetic elements adjacent to the indentations.

In some embodiments, the protrusions are at least partly ferromagnetic, and the chassis further includes respective magnetic elements adjacent to the indentations.

In some embodiments,
the cart includes a tray, which is configured to carry the console, and
the protrusions protrude upward from the tray.

In some embodiments,
the cart further includes a backstop, which is behind the tray,
the tray slants downward toward the backstop, and
the protrusions are spaced from the backstop so as to facilitate a backward tilt of the console while the console is on the tray.

There is further provided, in accordance with some embodiments, an apparatus for use with an intracorporeal device while the intracorporeal device is within a body of a subject. The apparatus includes a console, including a chassis, configured to connect to the intracorporeal device, a processor disposed within the chassis and configured to control the intracorporeal device via the connection, and a console hook coupled to the chassis. The apparatus further includes a cart configured to carry the console. The console is removable from the cart and is hangable, via the console hook, from a bedrail of a bed of the subject.

In some embodiments, the console hook is rotatably coupled to the chassis such that the console hook is rotatable from a closed position, in which the console hook does not protrude from the chassis, to an open position, in which the console hook protrudes from the chassis.

In some embodiments,
the chassis includes a ratchet and a release mechanism configured to release the ratchet, and
the console hook is coupled to the chassis via the ratchet such that:
   an activation of the release mechanism causes the console hook to rotate from the closed position to the open position, and
   following a rotation of the console hook from the open position to a partially-closed position in which the console hook secures the console on the bedrail, the ratchet maintains the console hook in the partially-closed position.

In some embodiments, the release mechanism includes a handle coupled to the ratchet, and the activation of the release mechanism includes a lifting of the handle such that, when a user lifts the handle so as to carry the console, via the handle, from the cart to the bedrail, the console hook rotates to the open position.

In some embodiments, the chassis is shaped to define one or more indentations, and the cart includes one or more protrusions configured to fit into the indentations while the cart carries the console.

In some embodiments, the protrusions are at least partly magnetic, and the chassis further includes respective ferromagnetic elements adjacent to the indentations.

In some embodiments, the protrusions are at least partly ferromagnetic, and the chassis further includes respective magnetic elements adjacent to the indentations.

In some embodiments,
the cart includes a tray, which is configured to carry the console, and
the protrusions protrude upward from the tray.

In some embodiments,
the cart further includes a backstop, which is behind the tray,
the tray slants downward toward the backstop, and
the protrusions are spaced from the backstop so as to facilitate a backward tilt of the console while the console is on the tray.

In some embodiments, the apparatus further includes a cartridge configured for insertion into the console such that the cartridge and the console interact with one another.

In some embodiments,
the intracorporeal device includes an inlet port, an outlet port, and a pressure-sensing port,
the apparatus is for use with:
   a purging-fluid bag containing purging fluid,
   a waste bag,
   a flushing-fluid bag containing flushing fluid, and
   a pressure sensor including a first sensor port and a second sensor port, the apparatus further includes:
   multiple tubes, including:
      a purging-fluid tube, configured to connect the purging-fluid bag to the inlet port of the intracorporeal device;
      a waste tube, configured to connect the waste bag to the outlet port of the intracorporeal device;
      a flushing tube, configured to connect the flushing-fluid bag to the first sensor port of the pressure sensor; and
      a pressure-sensing tube, configured to connect the second sensor port of the pressure sensor to the pressure-sensing port of the intracorporeal device such that
   the flushing fluid flows, via the pressure sensor, into the pressure-sensing port; and the cartridge holds the tubes.

In some embodiments,
the cartridge includes one or more pumps configured to pump the purging fluid from the purging-fluid bag, through the device, and into the waste bag, and
the console further includes one or more motors configured to drive the pumps following the insertion of the cartridge.

In some embodiments, the pumps include:
respective barrels, which are connected to the purging-fluid tube and/or the waste tube; and
respective plungers or pistons configured to reciprocate within the barrels, when driven by the motors, so as to pump the purging fluid.

In some embodiments, the pumps include respective rotors configured to squeeze the purging-fluid tube and/or the waste tube, when driven by the motors, so as to pump the purging fluid.

In some embodiments, the console further includes one or more pumps configured to pump the purging fluid from the purging-fluid bag, through the device, and into the waste bag following the insertion of the cartridge.

In some embodiments,
the pumps include respective rotors,
the cartridge is shaped to define one or more openings, and
the cartridge is configured for insertion into the console such that during the insertion, the rotors pass through the openings, respectively, such that the rotors are positioned to squeeze the purging-fluid tube and/or the waste tube so as to pump the purging fluid.

In some embodiments, the cartridge includes an electrical interface configured to receive electrical power from the console following the insertion of the cartridge.

In some embodiments,
the pressure sensor is a first pressure sensor, and
the console further includes a second pressure sensor configured to sense a pressure in the purging-fluid tube following the insertion of the cartridge.

In some embodiments, the purging-fluid tube includes an expandable portion within the cartridge, and the pressure sensor is configured to sense the pressure by sensing an expansion of the expandable portion.

In some embodiments, the console further includes a latch configured to close on the cartridge upon the insertion of the cartridge, such that the expansion of the expandable portion does not cause the cartridge to exit the console.

In some embodiments, the console includes a cable interface, and the apparatus further includes a cable, configured to connect the console to the intracorporeal device via the cable interface.

In some embodiments, the apparatus further includes a cart configured to carry the console and shaped to define:
a first groove, configured to hold the cable,
a second groove, configured to hold respective distal portions of the purging-fluid tube, the waste tube, and the pressure-sensing tube, and
a third groove, configured to hold a distal portion of the flushing tube and a proximal portion of the pressure-sensing tube.

In some embodiments, the cartridge includes a first port, a second port, and a third port, and the cartridge holds the tubes such that:
respective proximal portions of the purging-fluid tube, the waste tube, and the flushing tube pass through the first port,
the respective distal portions of the purging-fluid tube, the waste tube, and the pressure-sensing tube pass through the second port, and
the distal portion of the flushing tube and the proximal portion of the pressure-sensing tube pass through the third port.

In some embodiments,
the cartridge further includes an electrical interface configured to receive electrical power, and
the apparatus further includes an electric cable, which is connected to the electrical interface, exits the cartridge via the third port, and is configured to connect to the pressure sensor so as to deliver the electrical power to the pressure sensor.

In some embodiments, the cart is configured to carry the console such that, following the insertion of the cartridge:
the first groove is aligned with the cable interface,
the second groove is aligned with the second port, and
the third groove is aligned with the third port.

There is further provided, in accordance with some embodiments, an apparatus for use with one or more fluid bags configured to exchange fluid with a subject in a bed. The apparatus includes a cart and a cart adjunct, which is removably couplable to the cart. The cart adjunct includes one or more bag-holding appendages, configured to hold the fluid bags, and a coupling element. The cart adjunct is couplable, via the coupling element, to a bedrail of the bed.

In some embodiments, the coupling element includes a hook.

In some embodiments, the coupling element includes a clip.

In some embodiments,
the cart includes a post, shaped to define a post groove, and
the cart adjunct includes:
   a latch;
   a spring, configured to lock the cart adjunct to the post by pushing the latch into the post groove; and
   a latch release, configured to unlock the cart adjunct from the post by releasing the latch from the post groove.

In some embodiments, the latch release includes a slider coupled to the latch and configured to release the latch by sliding over the post.

In some embodiments, the slider is configured to release the latch by sliding upward, such that an upward sliding of the slider releases the latch and also lifts the cart adjunct from the cart.

In some embodiments,
the apparatus is for use with an intracorporeal device while the intracorporeal device is within a body of the subject,
the apparatus further includes a console, including:
   a chassis, configured to connect to the intracorporeal device;
   a processor disposed within the chassis and configured to control the intracorporeal device via the connection; and
   a console hook coupled to the chassis,
the cart is configured to carry the console, and
the console is removable from the cart and is hangable, via the console hook, from the bedrail.

In some embodiments, the chassis is shaped to define one or more indentations, and the cart includes one or more protrusions configured to fit into the indentations while the cart carries the console.

In some embodiments, the protrusions are at least partly magnetic, and the chassis further includes respective ferromagnetic elements adjacent to the indentations.

In some embodiments, the protrusions are at least partly ferromagnetic, and the chassis further includes respective magnetic elements adjacent to the indentations.

In some embodiments,
the cart includes a tray, which is configured to carry the console, and the protrusions protrude upward from the tray.

In some embodiments,
the cart further includes a backstop, which is behind the tray,
the tray slants downward toward the backstop, and
the protrusions are spaced from the backstop so as to facilitate a backward tilt of the console while the console is on the tray.

In some embodiments,
the intracorporeal device includes an inlet port, an outlet port, and a pressure-sensing port,
the fluid bags include a purging-fluid bag containing purging fluid, a waste bag, and a flushing-fluid bag containing flushing fluid,
the apparatus is for use with a pressure sensor including a first sensor port and a second sensor port, and
the apparatus further includes:
   multiple tubes, including:
      a purging-fluid tube, configured to connect the purging-fluid bag to the inlet port of the intracorporeal device;
      a waste tube, configured to connect the waste bag to the outlet port of the intracorporeal device;
      a flushing tube, configured to connect the flushing-fluid bag to the first sensor port of the pressure sensor; and
      a pressure-sensing tube, configured to connect the second sensor port of the pressure sensor to the pressure-sensing port of the intracorporeal device such that the flushing fluid flows, via the pressure sensor, into the pressure-sensing port; and a cartridge, which holds the tubes and is configured for insertion into the console such that the cartridge and the console interact with one another.

In some embodiments,
the cartridge includes one or more pumps configured to pump the purging fluid from the purging-fluid bag, through the device, and into the waste bag, and
the console includes one or more motors configured to drive the pumps following the insertion of the cartridge.

In some embodiments, the pumps include:
respective barrels, which are connected to the purging-fluid tube and/or the waste tube; and
respective plungers or pistons configured to reciprocate within the barrels, when driven by the motors, so as to pump the purging fluid.

In some embodiments, the pumps include respective rotors configured to squeeze the purging-fluid tube and/or the waste tube, when driven by the motors, so as to pump the purging fluid.

In some embodiments, the console further includes one or more pumps configured to pump the purging fluid from the purging-fluid bag, through the device, and into the waste bag following the insertion of the cartridge.

In some embodiments,
the pumps include respective rotors,
the cartridge is shaped to define one or more openings, and
the cartridge is configured for insertion into the console such that during the insertion, the rotors pass through the openings, respectively, such that the rotors are positioned to squeeze the purging-fluid tube and/or the waste tube so as to pump the purging fluid.

In some embodiments, the cartridge includes an electrical interface configured to receive electrical power from the console following the insertion of the cartridge.

In some embodiments,
the pressure sensor is a first pressure sensor, and
the console includes a second pressure sensor configured to sense a pressure in the purging-fluid tube following the insertion of the cartridge.

In some embodiments, the purging-fluid tube includes an expandable portion within the cartridge, and the pressure sensor is configured to sense the pressure by sensing an expansion of the expandable portion.

In some embodiments, the console further includes a latch configured to close on the cartridge upon the insertion of the cartridge, such that the expansion of the expandable portion does not cause the cartridge to exit the console.

In some embodiments, the console includes a cable interface, and the apparatus further includes a cable, configured to connect the console to the intracorporeal device via the cable interface.

In some embodiments, the cart is shaped to define:
a first groove, configured to hold the cable,
a second groove, configured to hold respective distal portions of the purging-fluid tube, the waste tube, and the pressure-sensing tube, and
a third groove, configured to hold a distal portion of the flushing tube and a proximal portion of the pressure-sensing tube.

In some embodiments, the cartridge includes a first port, a second port, and a third port, and the cartridge holds the tubes such that:
respective proximal portions of the purging-fluid tube, the waste tube, and the flushing tube pass through the first port,
the respective distal portions of the purging-fluid tube, the waste tube, and the pressure-sensing tube pass through the second port, and
the distal portion of the flushing tube and the proximal portion of the pressure-sensing tube pass through the third port.

In some embodiments,
the cartridge further includes an electrical interface configured to receive electrical power, and
the apparatus further includes an electric cable, which is connected to the electrical interface, exits the cartridge via the third port, and is configured to connect to the pressure sensor so as to deliver the electrical power to the pressure sensor.

In some embodiments, the cart is configured to carry the console such that, following the insertion of the cartridge:
the first groove is aligned with the cable interface,
the second groove is aligned with the second port, and
the third groove is aligned with the third port.

In some embodiments, the console hook is rotatably coupled to the chassis such that the console hook is rotatable from a closed position, in which the console hook does not protrude from the chassis, to an open position, in which the console hook protrudes from the chassis.

In some embodiments,
the chassis includes a ratchet and a release mechanism configured to release the ratchet, and
the console hook is coupled to the chassis via the ratchet such that:
   an activation of the release mechanism causes the console hook to rotate from the closed position to the open position, and
   following a rotation of the console hook from the open position to a partially-closed position in which the console hook secures the console on the bedrail, the ratchet maintains the console hook in the partially-closed position.

In some embodiments, the release mechanism includes a handle coupled to the ratchet, and the activation of the release mechanism includes a lifting of the handle such that, when a user lifts the handle so as to carry the console, via the handle, from the cart to the bedrail, the console hook rotates to the open position.

There is further provided, in accordance with some embodiments, an apparatus including a cart including a post shaped to define a post groove. The apparatus further includes a cart adjunct, which is removably couplable to the cart and includes a latch, a spring, configured to lock the cart adjunct to the post by pushing the latch into the post groove, and a slider, configured to release the latch from the post groove, thereby unlocking the cart adjunct from the post, by sliding upward over the post, such that an upward sliding of the slider releases the latch and also lifts the cart adjunct from the cart.

In some embodiments,
the apparatus is for use with one or more fluid bags configured to exchange fluid with a subject in a bed,
the cart adjunct further includes:
   one or more bag-holding appendages, configured to hold the fluid bags; and a coupling element, and
the cart adjunct is couplable, via the coupling element, on a bedrail of the bed.

In some embodiments,
the apparatus is for use with an intracorporeal device while the intracorporeal device is within a body of the subject,
the apparatus further includes a console, including:
   a chassis, configured to connect to the intracorporeal device;
   a processor disposed within the chassis and configured to control the intracorporeal device via the connection; and
   a console hook coupled to the chassis,
the cart is configured to carry the console, and
the console is removable from the cart and is hangable, via the console hook, from the bedrail.

There is further provided, in accordance with some embodiments, an apparatus including a left-ventricular assist device. The left-ventricular assist device includes a pump-outlet tube including a lateral wall shaped to define one or more blood-outlet openings and configured for insertion, through an aorta of a subject, into a left ventricle of a heart of the subject such that the blood-outlet openings are disposed within the aorta and a distal portion of the pump-outlet tube is disposed within the left ventricle. The left-ventricular assist device further includes an impeller configured to pump blood of the subject proximally through the pump-outlet tube, such that the blood exits the pump-outlet tube via the blood-outlet openings. The left-ventricular assist device further includes a delivery tube configured to extend, from outside a body of the subject, through the pump-outlet tube to the distal portion. The left-ventricular assist device further includes a drive cable passing through the delivery tube and configured to rotate the impeller. The left-ventricular assist device further includes an inflatable element that surrounds the delivery tube proximally to the blood-outlet openings and includes a distal inflatable-element portion, which is coupled to an inside of the lateral wall, and a proximal inflatable-element portion, which is wider than the pump-outlet tube and is disposed proximally to the pump-outlet tube.

In some embodiments, the distal inflatable-element portion is coupled to the inside of the lateral wall within 0.5-5 mm of the blood-outlet openings.

In some embodiments, the distal inflatable-element portion is coupled to the inside of the lateral wall within 1-3 mm of the blood-outlet openings.

In some embodiments, the proximal inflatable-element portion is configured to center the delivery tube within the aorta.

In some embodiments, the distal inflatable-element portion is at least partly cylindrical.

In some embodiments, the distal inflatable-element portion is shaped to direct the blood through the blood-outlet openings.

In some embodiments, a distal portion of the distal inflatable-element portion has a width that decreases moving distally.

In some embodiments, the distal portion of the distal inflatable-element portion is frustoconical.

In some embodiments, a wall of the delivery tube is shaped to define one or more inflation-fluid openings, and the inflatable element surrounds the inflation-fluid openings such that a fluid flowing, via the inflation-fluid openings, from the delivery tube into the inflatable element inflates the inflatable element.

In some embodiments,
the device further includes:
   an axial shaft coupled to the impeller and configured to rotate such that the impeller pumps the blood; and
   at least one bearing configured not to rotate with the axial shaft, and
distally to the inflation-fluid openings, the fluid purges an interface between the axial shaft and the bearing.

In some embodiments,
one or more flaps are cut in the lateral wall so as to define the blood-outlet openings, and
the distal inflatable-element portion is coupled to the flaps.

There is further provided, in accordance with some embodiments, an apparatus including a left-ventricular assist device. The left-ventricular assist device includes a pump-outlet tube including a lateral wall shaped to define one or more blood-outlet openings and configured for insertion, through an aorta of a subject, into a left ventricle of a heart of the subject such that the blood-outlet openings are disposed within the aorta and a distal portion of the pump-outlet tube is disposed within the left ventricle. The left-ventricular assist device further includes an impeller configured to pump blood of the subject proximally through the pump-outlet tube, such that the blood exits the pump-outlet tube via the blood-outlet openings. The left-ventricular assist device further includes a delivery tube configured to extend, from outside a body of the subject, through the pump-outlet tube to the distal portion. The left-ventricular assist device further includes a drive cable passing through the delivery tube and configured to rotate the impeller. The left-ventricular assist device further includes an inflatable element that surrounds the delivery tube proximally to the pump-outlet tube, a distal portion of the inflatable element being everted inwardly and coupled to the delivery tube.

In some embodiments, the inflatable element is configured to center the delivery tube within the aorta.

In some embodiments, a wall of the delivery tube is shaped to define one or more inflation-fluid openings, and the inflatable element surrounds the inflation-fluid openings such that a fluid flowing, via the inflation-fluid openings, from the delivery tube into the inflatable element inflates the inflatable element.

In some embodiments,
the device further includes:
   an axial shaft coupled to the impeller and configured to rotate such that the impeller pumps the blood; and
   at least one bearing configured not to rotate with the axial shaft, and
distally to the inflation-fluid openings, the fluid purges an interface between the axial shaft and the bearing.

In some embodiments, the pump-outlet tube includes a tubular coupling portion coupled to the delivery tube proximally to the blood-outlet openings, and the distal portion of the inflatable element is coupled to the delivery tube proximally to the tubular coupling portion at a distance of less than 20 mm from the tubular coupling portion.

In some embodiments, the distance is less than 10 mm.

There is further provided, in accordance with some embodiments, an apparatus including a left-ventricular assist device. The left-ventricular assist device includes a pump-outlet tube including a lateral wall shaped to define one or more blood-outlet openings and configured for insertion, through an aorta of a subject, into a left ventricle of a heart of the subject such that the blood-outlet openings are disposed within the aorta and a distal portion of the pump-outlet tube is disposed within the left ventricle. The left-ventricular assist device further includes an impeller configured to pump blood of the subject proximally through the pump-outlet tube, such that the blood exits the pump-outlet tube via the blood-outlet openings. The left-ventricular assist device further includes a delivery tube configured to extend, from outside a body of the subject, through the pump-outlet tube to the distal portion. The left-ventricular assist device further includes a drive cable passing through the delivery tube and configured to rotate the impeller. The left-ventricular assist device further includes an inflatable element that surrounds the delivery tube, is disposed at least partly within the pump-outlet tube proximally to the blood-outlet openings, and is coupled to the lateral wall within 0.5-5 mm of the blood-outlet openings.

In some embodiments, the inflatable element is coupled to the lateral wall within 1-3 mm of the blood-outlet openings.

In some embodiments, a distal portion of the inflatable element is shaped to direct the blood through the blood-outlet openings.

In some embodiments, the distal portion of the inflatable element has a width that decreases moving distally.

In some embodiments, the distal portion of the inflatable element is frustoconical.

In some embodiments, the inflatable element is configured to center the delivery tube within the aorta.

In some embodiments, a wall of the delivery tube is shaped to define one or more inflation-fluid openings, and the inflatable element surrounds the inflation-fluid openings such that a fluid flowing, via the inflation-fluid openings, from the delivery tube into the inflatable element inflates the inflatable element.

In some embodiments,
the device further includes:
   an axial shaft coupled to the impeller and configured to rotate such that the impeller pumps the blood; and
   at least one bearing configured not to rotate with the axial shaft, and
distally to the inflation-fluid openings, the fluid purges an interface between the axial shaft and the bearing.

In some embodiments, the inflatable element includes:
a distal inflatable-element portion, which is disposed within the pump-outlet tube proximally to the blood-outlet openings, and is coupled to the lateral wall within 0.5-5 mm of the blood-outlet openings; and
a proximal inflatable-element portion, which is wider than the pump-outlet tube and is disposed proximally to the pump-outlet tube.

In some embodiments, the distal inflatable-element portion is at least partly cylindrical.

In some embodiments,
one or more flaps are cut in the lateral wall so as to define the blood-outlet openings, and
the inflatable element is coupled to the flaps.

There is further provided, in accordance with some embodiments, an apparatus including a left-ventricular assist device. The left-ventricular assist device includes a pump-outlet tube, which includes a lateral wall in which flaps are cut so as to define one or more blood-outlet openings, and which is configured for insertion, through an aorta of a subject, into a left ventricle of a heart of the subject such that the blood-outlet openings are disposed within the aorta and a distal portion of the pump-outlet tube is disposed within the left ventricle. The left-ventricular assist device further includes an impeller configured to pump blood of the subject proximally through the pump-outlet tube, such that the blood exits the pump-outlet tube via the blood-outlet openings. The left-ventricular assist device further includes a delivery tube configured to extend, from outside a body of the subject, through the pump-outlet tube to the distal portion. The left-ventricular assist device further includes a drive cable passing through the delivery tube and configured to rotate the impeller. The left-ventricular assist device further includes an inflatable element that surrounds the delivery tube, is disposed at least partly within the pump-outlet tube proximally to the blood-outlet openings, and is coupled to the flaps.

In some embodiments, the inflatable element is shaped to direct the blood through the blood-outlet openings.

In some embodiments, the inflatable element is configured to center the delivery tube within the aorta.

In some embodiments, a wall of the delivery tube is shaped to define one or more inflation-fluid openings, and the inflatable element surrounds the inflation-fluid openings such that a fluid flowing, via the inflation-fluid openings, from the delivery tube into the inflatable element inflates the inflatable element.

In some embodiments,
the device further includes:
   an axial shaft coupled to the impeller and configured to rotate such that the impeller pumps the blood; and
   at least one bearing configured not to rotate with the axial shaft, and
distally to the inflation-fluid openings, the fluid purges an interface between the axial shaft and the bearing.

In some embodiments, the inflatable element is coupled to the lateral wall within 0.5-5 mm of the blood-outlet openings.

In some embodiments, the inflatable element is coupled to the lateral wall within 1-3 mm of the blood-outlet openings.

In some embodiments, the inflatable element includes:
a distal inflatable-element portion, which is coupled to an inside of the lateral wall; and
a proximal inflatable-element portion, which is wider than the pump-outlet tube and is disposed proximally to the pump-outlet tube.

In some embodiments, the distal inflatable-element portion is at least partly cylindrical.

In some embodiments,
the pump-outlet tube further includes multiple tabs extending proximally from a proximal portion of the pump-outlet tube,
the inflatable element is shaped to define one or more grooves, and
the tabs are coupled to the delivery tube proximally to the inflatable element.

In some embodiments, the inflatable element is shaped to define three grooves.

In some embodiments, the inflatable element is shaped to define four grooves.

In some embodiments, the inflatable element is shaped to define 6-10 grooves.

In some embodiments, the tabs pass through the grooves.

In some embodiments, the tabs are coupled to the delivery tube immediately proximally to the inflatable element.

In some embodiments, the inflatable element is disposed within the proximal portion of the pump-outlet tube, and at least some of the blood exits the proximal portion of the pump-outlet tube via the grooves.

In some embodiments, the tabs are coupled to the delivery tube 0.5-6 mm from the inflatable element.

In some embodiments, the tabs are coupled to the delivery tube 1-3 mm from the inflatable element.

There is further provided, in accordance with some embodiments, an apparatus including a left-ventricular assist device. The left-ventricular assist device includes a pump-outlet tube, including a lateral wall shaped to define one or more blood-outlet openings and multiple tabs extending proximally from a proximal portion of the pump-outlet tube. The pump-outlet tube is configured for insertion, through an aorta of a subject, into a left ventricle of a heart of the subject such that the blood-outlet openings are disposed within the aorta and a distal portion of the pump-outlet tube is disposed within the left ventricle. The left-ventricular assist device further includes an impeller configured to pump blood of the subject proximally through the pump-outlet tube, such that the blood exits the pump-outlet tube via the blood-outlet openings. The left-ventricular assist device further includes a delivery tube configured to extend, from outside a body of the subject, through the pump-outlet tube to the distal portion. The left-ventricular assist device further includes a drive cable passing through the delivery tube and configured to rotate the impeller. The left-ventricular assist device further includes an inflatable element that surrounds the delivery tube and is disposed at least partly within the pump-outlet tube proximally to the blood-outlet openings, the tabs being coupled to the delivery tube proximally to the inflatable element.

In some embodiments, the inflatable element is shaped to direct the blood through the blood-outlet openings.

In some embodiments, the inflatable element is configured to center the delivery tube within the aorta.

In some embodiments, a wall of the delivery tube is shaped to define one or more inflation-fluid openings, and the inflatable element surrounds the inflation-fluid openings such that a fluid flowing, via the inflation-fluid openings, from the delivery tube into the inflatable element inflates the inflatable element.

In some embodiments,
the device further includes:
   an axial shaft coupled to the impeller and configured to rotate such that the impeller pumps the blood; and
   at least one bearing configured not to rotate with the axial shaft, and
distally to the inflation-fluid openings, the fluid purges an interface between the axial shaft and the bearing.

In some embodiments, the inflatable element is coupled to the lateral wall within 0.5-5 mm of the blood-outlet openings.

In some embodiments, the inflatable element is coupled to the lateral wall within 1-3 mm of the blood-outlet openings.

In some embodiments, the inflatable element is shaped to define one or more grooves, and the tabs pass through the grooves.

In some embodiments, the inflatable element is shaped to define three grooves.

In some embodiments, the inflatable element is shaped to define four grooves.

In some embodiments, the inflatable element is shaped to define 6-10 grooves.

In some embodiments, the tabs are coupled to the delivery tube immediately proximally to the inflatable element.

There is further provided, in accordance with some embodiments, an apparatus including a left-ventricular assist device. The left-ventricular assist device includes a pump-outlet tube, which includes a distal portion, a proximal portion, and multiple tabs extending proximally from the proximal portion, and which is configured for insertion, through an aorta of a subject, into a left ventricle of a heart of the subject such that the proximal portion is disposed within the aorta and the distal portion is disposed within the left ventricle. The left-ventricular assist device further includes an impeller configured to pump blood of the subject proximally through the pump-outlet tube, such that the blood exits the proximal portion of the pump-outlet tube. The left-ventricular assist device further includes a delivery tube configured to extend, from outside a body of the subject, through the pump-outlet tube to the distal portion. The left-ventricular assist device further includes a drive cable passing through the delivery tube and configured to rotate the impeller. The left-ventricular assist device further includes an inflatable element that surrounds the delivery tube and is shaped to define one or more grooves, the tabs being coupled to the delivery tube proximally to the inflatable element.

In some embodiments, the inflatable element is shaped to define three grooves.

In some embodiments, the inflatable element is shaped to define four grooves.

In some embodiments, the inflatable element is shaped to define 6-10 grooves.

In some embodiments, the inflatable element is configured to center the delivery tube within the aorta.

In some embodiments, a wall of the delivery tube is shaped to define one or more inflation-fluid openings, and the inflatable element surrounds the inflation-fluid openings such that a fluid flowing, via the inflation-fluid openings, from the delivery tube into the inflatable element inflates the inflatable element.

In some embodiments,
the left-ventricular assist device further includes:
   an axial shaft coupled to the impeller and configured to rotate such that the impeller pumps the blood; and
   at least one bearing configured not to rotate with the axial shaft, and
distally to the inflation-fluid openings, the fluid purges an interface between the axial shaft and the bearing.

In some embodiments, the tabs pass through the grooves.

In some embodiments, the tabs are coupled to the delivery tube immediately proximally to the inflatable element.

In some embodiments, the proximal portion of the pump-outlet tube includes a lateral wall shaped to define one or more blood-outlet openings, and the blood exits the proximal portion of the pump-outlet tube via the blood-outlet openings.

In some embodiments, flaps are cut in the lateral wall so as to define the blood-outlet openings, and the flaps are coupled to the inflatable element.

In some embodiments, the inflatable element is shaped to direct the blood through the blood-outlet openings.

In some embodiments, the inflatable element contacts the proximal portion of the blood-outlet tube.

In some embodiments,
the inflatable element is offset proximally from the proximal portion of the pump-outlet tube so as to define multiple blood-outlet openings between the tabs and between the proximal portion of the pump-outlet tube and the inflatable element, and
the blood exits the proximal portion of the pump-outlet tube via the blood-outlet openings.

In some embodiments, the inflatable element is shaped to direct the blood through the blood-outlet openings.

In some embodiments, the inflatable element is disposed within the proximal portion of the pump-outlet tube, and at least some of the blood exits the proximal portion of the pump-outlet tube via the grooves.

In some embodiments, the tabs are coupled to the delivery tube 0.5-6 mm from the inflatable element.

In some embodiments, the tabs are coupled to the delivery tube 1-3 mm from the inflatable element.

In some embodiments, the proximal portion of the pump-outlet tube includes a lateral wall shaped to define one or more blood-outlet openings, and some of the blood exits the proximal portion of the pump-outlet tube via the blood-outlet openings.

In some embodiments, flaps are cut in the lateral wall so as to define the blood-outlet openings, and the flaps are coupled to the inflatable element.

There is further provided, in accordance with some embodiments, a method for manufacturing a blood pump. The method includes inserting an impeller, which is configured to pump blood of a subject, into a frame including a proximal portion, a central portion, and a frustoconical distal portion. The method further includes fixing a pump-outlet tube over the proximal portion of the frame and at least part of the central portion of the frame. The method further includes forming one or more blood-inlet openings in a sheet of material, and subsequently to forming the blood-inlet openings, rolling the sheet of material so as to form a frustoconical inlet guard. The method further includes coupling the inlet guard to the pump-outlet tube, and fixing the inlet guard over the distal portion of the frame.

In some embodiments,
one or more tabs extend from the sheet of material,
rolling the sheet of material includes rolling the sheet of material by pulling the tabs, and
the method further includes, subsequently to rolling the sheet of material, removing the tabs from the sheet of material.

In some embodiments, the pump-outlet tube is configured to traverse an aortic valve of the subject, and the impeller is configured to pump the blood from a left ventricle of a heart of the subject, through the pump-outlet tube, into an aorta of the subject.

In some embodiments, the blood-inlet openings are sized such that the inlet guard is configured to block chordae tendineae, trabeculae carneae, and papillary muscles of a left ventricle of a heart of the subject from entering the frame.

In some embodiments, for each of the blood-inlet openings, a span of the blood-inlet opening in at least one direction is less than 1 mm.

In some embodiments, an area of each of the blood-inlet openings is 0.05-5 mm².

In some embodiments, forming the blood-inlet openings includes forming the blood-inlet openings such that a porosity of the inlet guard is at least 40 percent.

In some embodiments, each of the blood-inlet openings is hexagonal.

In some embodiments, forming the blood-inlet openings includes forming the blood-inlet openings such that a distance between each pair of adjacent ones of the blood-inlet openings is 0.01-0.1 mm.

In some embodiments, fixing the inlet guard over the distal portion of the frame includes fixing the inlet guard over the distal portion of the frame by coupling the inlet guard to the distal portion of the frame.

In some embodiments, the method further includes forming multiple proximal flaps in the sheet of material, and coupling the inlet guard to the pump-outlet tube includes coupling the inlet guard to the pump-outlet tube by coupling the proximal flaps to the pump-outlet tube.

In some embodiments, fixing the inlet guard over the distal portion of the frame includes fixing the inlet guard over the distal portion of the frame by fixing the proximal flaps at least partly over the central portion of the frame.

In some embodiments, the central portion of the frame includes multiple struts, and forming the proximal flaps includes shaping the proximal flaps such that, when the proximal flaps are fixed at least partly over the central portion of the frame, the proximal flaps do not overlap any of the struts.

In some embodiments, shaping the proximal flaps includes shaping the proximal straps such that, when the proximal flaps are fixed at least partly over the central portion of the frame, the proximal flaps fit between the struts while abutting the struts.

In some embodiments, the method further includes:
lining at least part of the central portion of the frame with an inner lining; and
coupling the inlet guard to the inner lining.

In some embodiments, the method further includes forming multiple proximal flaps in the sheet of material,
coupling the inlet guard to the pump-outlet tube includes coupling the inlet guard to the pump-outlet tube by coupling the proximal flaps to the pump-outlet tube, and
coupling the inlet guard to the inner lining includes coupling the inlet guard to the inner lining by coupling the proximal flaps to the inner lining, such that the proximal flaps are between the pump-outlet tube and the inner lining.

In some embodiments, coupling the proximal flaps to the pump-outlet tube and to the inner lining includes coupling the proximal flaps to the pump-outlet tube and to the inner lining by heat welding the pump-outlet tube to the inner lining while the proximal flaps are between the pump-outlet tube and the inner lining.

In some embodiments, the method further includes forming multiple flap openings in the proximal flaps, and heat welding the pump-outlet tube to the inner lining includes heat welding the pump-outlet tube to the inner lining at least partly via the flap openings.

In some embodiments, a glass-transition temperature of the material is higher than respective glass-transition temperatures of each of the inner lining and the pump-outlet tube, and heat welding the pump-outlet tube to the inner lining includes heat welding the pump-outlet tube to the inner lining at a heat-welding temperature that is lower than the glass-transition temperature of the material but higher than the glass-transition temperatures of each of the inner lining and the pump-outlet tube.

In some embodiments,
the inner lining is made of a polyurethane,
the pump-outlet tube is made of a polyether block amide, and
the material is a polyether ether ketone.

In some embodiments,
the inner lining and the pump-outlet tube are made of the same type or different types of polyurethane, and
the material is a polyether ether ketone.

In some embodiments,
inserting the impeller into the frame includes inserting the impeller into the frame while the impeller is disposed over an axial shaft configured to rotate,
the method further includes coupling a bearing housing, which houses a radial bearing configured to radially stabilize the axial shaft while the axial shaft rotates, to the frame distally to the frame, and
fixing the inlet guard over the distal portion of the frame includes fixing the inlet guard over the distal portion of the frame by coupling the inlet guard to the bearing housing.

In some embodiments, the method further includes forming multiple distal flaps in the sheet of material, and coupling the inlet guard to the bearing housing includes coupling the inlet guard to the bearing housing by coupling the distal flaps to the bearing housing.

There is further provided, in accordance with some embodiments, an apparatus including a blood pump. The blood pump includes a frame including a proximal portion, a central portion, and a frustoconical distal portion. The blood pump further includes an impeller, which is configured to pump blood of a subject, disposed within the frame. The blood pump further includes a pump-outlet tube, which is fixed over the proximal portion of the frame and at least part of the central portion of the frame. The blood pump further includes a frustoconical inlet guard, which includes a rolled sheet of material shaped to define one or more blood-inlet openings, which is coupled to the pump-outlet tube, and which is fixed over the distal portion of the frame.

There is further provided, in accordance with some embodiments, an apparatus including a blood pump. The blood pump includes a frame including a proximal portion, a central portion, and a distal portion. The blood pump further includes an inner lining that lines at least part of the central portion of the frame, an impeller, which is configured to pump blood of a subject proximally, disposed within the frame, and a pump-outlet tube, which is fixed over the proximal portion of the frame and at least part of the central portion of the frame, and which is heat welded to the inner lining. The blood pump further includes an inlet guard, which is distal to the impeller, includes a main body shaped to define one or more blood-inlet openings configured to allow passage of the blood therethrough, includes multiple proximal flaps extending proximally from the main body and disposed between the pump-outlet tube and the inner lining where the pump-outlet tube and the inner lining are heat welded to one another, and is made of a material having a glass-transition temperature that is higher than respective glass-transition temperatures of each of the inner lining and the pump-outlet tube.

In some embodiments, the proximal flaps are shaped to define multiple flap openings, and the pump-outlet tube and the inner lining are heat welded to one another at least partly via the flap openings.

In some embodiments,
the inner lining is made of a polyurethane,
the pump-outlet tube is made of a polyether block amide, and
the material is a polyether ether ketone.

In some embodiments,
the inner lining and the pump-outlet tube are made of the same type or different types of polyurethane, and
the material is a polyether ether ketone.

In some embodiments, the pump-outlet tube is configured to traverse an aortic valve of the subject, and the impeller is configured to pump the blood from a left ventricle of a heart of the subject, through the pump-outlet tube, into an aorta of the subject.

In some embodiments, the blood-inlet openings are sized such that the inlet guard is configured to block chordae tendineae, trabeculae carneae, and papillary muscles of a left ventricle of a heart of the subject from entering the frame.

In some embodiments, for each of the blood-inlet openings, a span of the blood-inlet opening in at least one direction is less than 1 mm.

In some embodiments, an area of each of the blood-inlet openings is 0.05-5 mm².

In some embodiments, a porosity of the inlet guard is at least 40 percent.

In some embodiments, each of the blood-inlet openings is hexagonal.

In some embodiments, a distance between each pair of adjacent ones of the blood-inlet openings is 0.01-0.1 mm.

In some embodiments, the inlet guard is coupled to the distal portion of the frame.

In some embodiments, the proximal flaps are fixed at least partly over the central portion of the frame.

In some embodiments, the central portion of the frame includes multiple struts, and the proximal flaps do not overlap any of the struts.

In some embodiments, the proximal flaps fit between the struts while abutting the struts.

In some embodiments, the apparatus further includes:
an axial shaft configured to rotate;
a radial bearing configured to radially stabilize the axial shaft while the axial shaft rotates; and
a bearing housing, which houses the radial bearing and is coupled to the frame distally to the frame,
the impeller is disposed over the axial shaft, and
the inlet guard is coupled to the bearing housing.

In some embodiments, the inlet guard includes multiple distal flaps, which are coupled to the bearing housing.

In some embodiments,
the distal portion of the frame is frustoconical, and
the main body of the inlet guard is frustoconical and is fixed over the distal portion of the frame.

In some embodiments, the inlet guard includes a rolled sheet of the material.

In some embodiments, the main body of the inlet guard is flat and is disposed within the frame.

In some embodiments, the apparatus further includes an axial shaft configured to rotate,
the impeller is disposed over the axial shaft, and
the main body of the inlet guard is perpendicular to the axial shaft.

There is further provided, in accordance with some embodiments, an apparatus including a blood pump. The blood pump includes a frame including a proximal portion, a central portion including multiple struts, and a distal portion. The blood pump further includes an impeller, which is configured to pump blood of a subject proximally, disposed within the frame. The blood pump further includes a pump-outlet tube, which is fixed over the proximal portion of the frame and at least part of the central portion of the frame. The blood pump further includes an inlet guard, which is distal to the impeller, includes a main body shaped to define one or more blood-inlet openings configured to allow passage of the blood therethrough, and includes multiple proximal flaps, which are coupled to the pump-outlet tube and are fixed at least partly over the central portion of the frame without overlapping any of the struts.

In some embodiments, the proximal flaps fit between the struts while abutting the struts.

In some embodiments, the pump-outlet tube is configured to traverse an aortic valve of the subject, and the impeller is configured to pump the blood from a left ventricle of a heart of the subject, through the pump-outlet tube, into an aorta of the subject.

In some embodiments, the blood-inlet openings are sized such that the inlet guard is configured to block chordae tendineae, trabeculae carneae, and papillary muscles of a left ventricle of a heart of the subject from entering the frame.

In some embodiments, for each of the blood-inlet openings, a span of the blood-inlet opening in at least one direction is less than 1 mm.

In some embodiments, an area of each of the blood-inlet openings is 0.05-5 mm².

In some embodiments, a porosity of the inlet guard is at least 40 percent.

In some embodiments, each of the blood-inlet openings is hexagonal.

In some embodiments, a distance between each pair of adjacent ones of the blood-inlet openings is 0.01-0.1 mm.

In some embodiments, the inlet guard is coupled to the distal portion of the frame.

In some embodiments, the apparatus further includes an inner lining that lines at least part of the central portion of the frame and is coupled to the inlet guard.

In some embodiments, the proximal flaps are coupled to the inner lining.

In some embodiments, the pump-outlet tube and the inner lining are heat welded to one another with the proximal flaps being disposed between the pump-outlet tube and the inner lining.

In some embodiments, the proximal flaps are shaped to define multiple flap openings, and the pump-outlet tube and the inner lining are heat welded to one another at least partly via the flap openings.

In some embodiments, a glass-transition temperature of the inlet guard is higher than the respective glass-transition temperatures of each of the inner lining and the pump-outlet tube.

In some embodiments,
the inner lining is made of a polyurethane,
the pump-outlet tube is made of a polyether block amide, and
the inlet guard is made of a polyether ether ketone.

In some embodiments,
the inner lining and the pump-outlet tube are made of the same type or different types of polyurethane, and
the inlet guard is made of a polyether ether ketone.

In some embodiments, the apparatus further includes:
an axial shaft configured to rotate;
a radial bearing configured to radially stabilize the axial shaft while the axial shaft rotates; and
a bearing housing, which houses the radial bearing and is coupled to the frame distally to the frame,
the impeller is disposed over the axial shaft, and
the inlet guard is coupled to the bearing housing.

In some embodiments, the inlet guard includes multiple distal flaps, which are coupled to the bearing housing.

In some embodiments,
the distal portion of the frame is frustoconical, and
the main body of the inlet guard is frustoconical and is fixed over the distal portion of the frame.

In some embodiments, the inlet guard includes a rolled sheet of material.

In some embodiments, the main body of the inlet guard is flat and is disposed within the frame.

In some embodiments, the apparatus further includes an axial shaft configured to rotate,
the impeller is disposed over the axial shaft, and p1 the main body of the inlet guard is perpendicular to the axial shaft.

There is further provided, in accordance with some embodiments, apparatus including:
a blood pump, including:
   a frame;
   an inner lining, which has a flexural modulus of between 0.1GPa and 2 GPa and lines at least part of the frame;
   an impeller, which is configured to pump blood of a subject, disposed within the frame; and
   a pump-outlet tube, which has a flexural modulus of between 0.1 GPa and 0.8 GPa, is fixed over at least part of the frame, is heat welded to the inner lining,
      respective melting temperatures of the inner lining and the pump-outlet tube being within 20 °C of one another.

In some embodiments, the inner lining and the pump-outlet tube are made of the same material, such that the melting temperatures are the same as one another.

In some embodiments, the flexural modulus of the inner lining is between 0.1 GPa and 0.8 GPa.

In some embodiments, the flexural modulus of the inner lining is between 0.8 GPa and 2 GPa.

In some embodiments, the flexural modulus of the pump-outlet tube is between 0.1 GPa and 0.5 GPa.

In some embodiments, the inner lining is made of a first polymer and the pump-outlet tube is made of a second polymer and the first and second polymers belong to a single class of polymer.

In some embodiments, the first polymer and the second polymer are the same as each other.

In some embodiments, the first polymer and the second polymer are different polymers from each other.

In some embodiments, the first and second polymers are both polyurethanes.

In some embodiments, the respective melting temperatures of the inner lining and the pump-outlet tube are within 10 °C of one another.

In some embodiments, the respective melting temperatures of the inner lining and the pump-outlet tube are within 5 °C of one another.

In some embodiments, the respective melting temperatures of the inner lining and the pump-outlet tube are the same as one another.

In some embodiments, respective glass-transition temperatures of the inner lining and the pump-outlet tube are within 20 °C of one another.

In some embodiments, the respective glass-transition temperatures of the inner lining and the pump-outlet tube are within 10 °C of one another.

In some embodiments, the respective glass-transition temperatures of the inner lining and the pump-outlet tube are within 5 °C of one another.

In some embodiments, the respective glass-transition temperatures of the inner lining and the pump-outlet tube are the same as one another.

There is further provided, in accordance with some embodiments, including:
a blood pump, including:
   a frame;
   an inner lining, made of a first polymer that has a flexural modulus of between 0.1GPa and 2 GPa and lines at least part of the frame;
   an impeller, which is configured to pump blood of a subject, disposed within the frame; and
   a pump-outlet tube, which is made of a second polymer that has a flexural modulus of between 0.1 GPa and 0.8 GPa, is fixed over at least part of the frame, is heat welded to the inner lining,
      the first and second polymers belong to a single class of polymer.

In some embodiments, the flexural modulus of the inner lining is between 0.1 GPa and 0.8 GPa.

In some embodiments, the flexural modulus of the inner lining is between 0.8 GPa and 2 GPa.

In some embodiments, the flexural modulus of the pump-outlet tube is between 0.1 GPa and 0.5 GPa.

In some embodiments, the first polymer and the second polymer are the same as each other.

In some embodiments, the first polymer and the second polymer are different polymers from each other.

In some embodiments, the first and second polymers are both polyurethanes.

In some embodiments, the respective melting temperatures of the inner lining and the pump-outlet tube are within 20 °C of one another.

In some embodiments, the respective melting temperatures of the inner lining and the pump-outlet tube are within 10 °C of one another.

In some embodiments, the respective melting temperatures of the inner lining and the pump-outlet tube are within 5 °C of one another.

In some embodiments, the respective melting temperatures of the inner lining and the pump-outlet tube are the same as one another.

In some embodiments, respective glass-transition temperatures of the inner lining and the pump-outlet tube are within 20 °C of one another.

In some embodiments, the respective glass-transition temperatures of the inner lining and the pump-outlet tube are within 10 °C of one another.

In some embodiments, the respective glass-transition temperatures of the inner lining and the pump-outlet tube are within 5 °C of one another.

In some embodiments, the respective glass-transition temperatures of the inner lining and the pump-outlet tube are the same as one another.

The present disclosure will be more fully understood from the following detailed description of embodiments thereof, taken together with the drawings, in which:

### BRIEF DESCRIPTION OF THE DRAWINGS

FIG. 1A is a schematic illustration of a ventricular assist system, in accordance with some embodiments;

FIG. 1B schematically illustrates the deployment of a ventricular assist device within the left ventricle of a subject, in accordance with some embodiments;

FIGS. 1C and 1D are schematic illustrations of a pump-head portion of a ventricular assist device, in accordance with some embodiments;

FIG. 2 is a schematic illustration of a frame that houses an impeller of a ventricular assist device, in accordance with some embodiments;

FIGS. 3A and 3B are schematic illustrations of an impeller of a ventricular assist device, in accordance with some embodiments;

FIG. 4 is a schematic illustration of an impeller disposed inside the frame of a ventricular assist device, in accordance with some embodiments;

FIGS. 5A and 5B are schematic illustrations of the impeller and the frame of the ventricular assist device, respectively in non-radially-constrained and radially-constrained states thereof, in accordance with some embodiments;

FIG. 5C is an enlarged schematic illustration of the proximal end of the frame of the ventricular assist device, in accordance with some embodiments;

FIGS. 5D, 5E, and 5F are schematic illustrations of a coupling element, in accordance with some embodiments;

FIGS. 6A, 6B, and 6C are schematic illustrations of a ventricular assist device that includes an inner lining on the inside of the frame that houses the impeller of a ventricular assist device, in accordance with some embodiments;

FIGS. 7A, 7B, 7C, and 7D are schematic illustrations of a pump-outlet tube that defines blood-inlet openings at a distal end thereof, in accordance with some embodiments;

FIG. 8A is a schematic illustration of a sheet of material, in accordance with some embodiments;

FIG. 8B is a schematic illustration of a frustoconical inlet guard formed from the sheet shown in FIG. 8A, in accordance with some embodiments;

FIG. 8C is a schematic illustration of a sheet of material, in accordance with some embodiments;

FIG. 8D is a schematic illustration of a frustoconical inlet guard fixed over the distal portion of a frame, in accordance with some embodiments;

FIG. 9A is a schematic illustration of an expandable element surrounding a delivery tube, in accordance with some embodiments;

FIG. 9B is a schematic illustration of a pump-head portion of a ventricular assist device, in accordance with some embodiments;

FIGS. 10A, 10B, 10C, and 10D are schematic illustrations of a pump-head portion of a ventricular assist device, in accordance with some embodiments;

FIG. 10E is a schematic illustration of a pump-outlet tube that defines a blood-flow chamber at its proximal end, in accordance with some embodiments;

FIG. 10F is a schematic illustration of an inflatable element disposed at the proximal end of a pump-outlet tube, in accordance with some embodiments;

FIG. 10G is a schematic illustration of an inflatable element disposed proximally to a pump-outlet tube, in accordance with some embodiments;

FIG. 10H is a schematic illustration of a pump-head portion of a ventricular assist device, in accordance with some embodiments;

FIGS. 10I and 10J are schematic illustrations of an inflatable element from oblique and frontal perspectives, respectively, in accordance with some embodiments;

FIGS. 10K, 10L, 10M, and 10N are schematic illustrations of a pump-head portion of a ventricular assist device, in accordance with some embodiments.

FIG. 10O shows a schematic frontal view of the proximal end of a pump-head portion of a ventricular assist device, in accordance with some embodiments;

FIG. 10P is a schematic illustration of a pump-head portion of a ventricular assist device, in accordance with some embodiments;

FIGS. 11A, 11B, 11C, and 11D are schematic illustrations of portions of a ventricular assist device, in accordance with some embodiments;

FIG. 11E is a schematic illustration of an inlet guard, in accordance with some embodiments;

FIG. 11F is a schematic illustration of an inlet guard disposed within a frame, in accordance with some embodiments;

FIG. 12A is a schematic illustration of a ventricular assist device packaged within packaging, in accordance with some embodiments of the present invention;

FIGS. 12B and 12C schematically illustrate a method for removing the ventricular assist device from the packaging, in accordance with some embodiments of the present invention;

FIG. 13 is a schematic illustration of packaging for a ventricular assist device, in accordance with some embodiments of the present invention;

FIG. 14 is a schematic illustration of a cart for use with an intracorporeal device, in accordance with some embodiments;

FIGS. 15A and 15B are schematic layouts of a cartridge and associated components, in accordance with some embodiments;

FIG. 16A is a schematic illustration of a console as viewed from behind, in accordance with some embodiments;

FIG. 16B is a schematic illustration of a console as viewed from the side, in accordance with some embodiments;

FIG. 16C is a schematic illustration of a console, in accordance with some embodiments;

FIG. 16D is a schematic illustration of a portion of a cart, in accordance with some embodiments;

FIG. 17A schematically shows several enlargements of a portion of a cart adjunct and post, in accordance with some embodiments;

FIG. 17B is a schematic illustration of a cart adjunct and console hanging from a bedrail, in accordance with some embodiments;

FIG. 18A is a schematic illustration of a guide for facilitating repositioning an intracorporeal device within a body of a subject, in accordance with some embodiments; and

FIG. 18B shows a schematic longitudinal cross-section through a guide and driven-magnet unit, in accordance with some embodiments.

### DETAILED DESCRIPTION

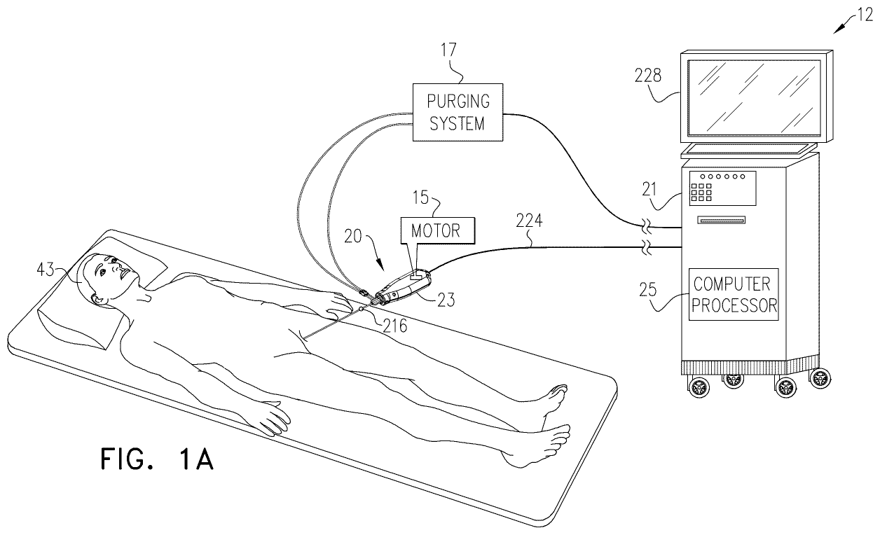

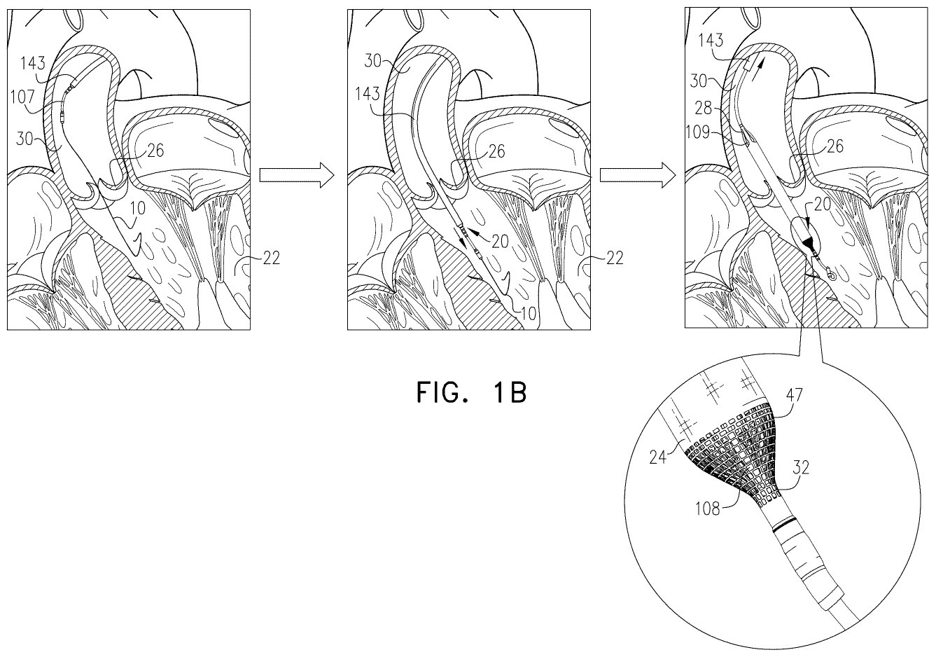

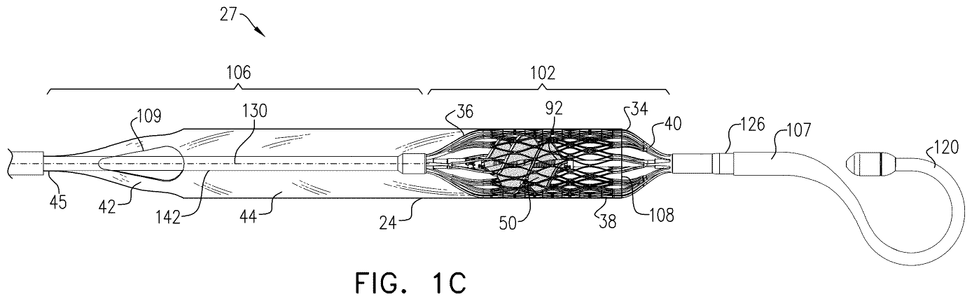

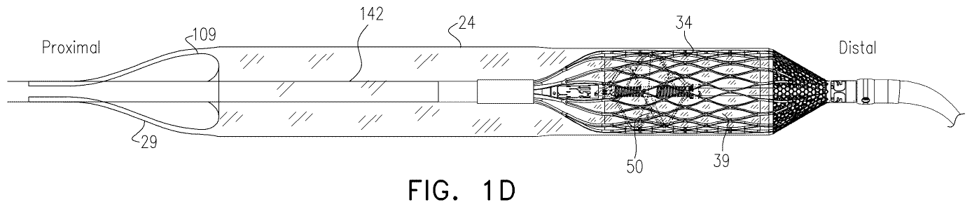

Reference is initially made to FIG. 1A, which is a schematic illustration of a ventricular assist system 12 comprising a ventricular assist device 20 configured to assist left-ventricular function of a subject 43, in accordance with some embodiments. Reference is also made to FIG. 1B, which schematically illustrates the deployment of device 20 within the left ventricle 22 of subject 43, in accordance with some embodiments. Reference is additionally made to FIG. 1C, which is a schematic illustration of a pump-head portion 27 of device 20, in accordance with some embodiments. Given that the scope of the present disclosure includes using the apparatus and methods described herein in anatomical locations other than the left ventricle and the aorta, ventricular assist device 20 and/or portions thereof are sometimes referred to herein (in the specification and the claims) as a blood pump.

Ventricular assist device 20 comprises a pump-outlet tube 24, which is shaped to define one or more blood-outlet openings 109. Typically, a proximal section 106 of the pump-outlet tube defines blood-outlet openings 109 such that the blood-outlet openings are near the proximal end 28 of pump-outlet tube 24. The pump-outlet tube is configured for insertion, through the aorta 30 of subject 43, into left ventricle 22 such that, by virtue of the pump-outlet tube traversing the aortic valve 26 of the subject, blood-outlet openings 109 are disposed within the aorta and a distal section 102 of the pump-outlet tube, which includes the distal end 32 of the pump-outlet tube, is disposed within the left ventricle. Pump-outlet tube 24 (which may also be referred to as a “blood-pump tube”) is typically an elongate tube, an axial length of the pump-outlet tube typically being substantially larger than its diameter.

The ventricular assist device further comprises an impeller 50, which in some embodiments is disposed within distal section 102. Impeller 50 is configured to pump blood of the subject proximally through the pump-outlet tube such that the blood exits the pump-outlet tube via blood-outlet openings 109. Thus, during operation of impeller 50, blood flows from the pump-outlet tube into the ascending aorta.

The ventricular assist device further comprises a delivery tube 142 configured to extend, from outside the body of the subject, through the pump-outlet tube to the distal section of the pump-outlet tube. The device further comprises a drive cable 130 passing through the delivery tube and operatively coupled to impeller 50. System 12 further comprises a motor unit 23, which comprises a motor 15 configured to rotate the impeller via drive cable 130.

The pump-outlet tube typically defines one or more blood-inlet openings 108 at the distal end of the pump-outlet tube, via which blood flows into the pump-outlet tube, from the left ventricle, during operation of the impeller. As shown in FIG. 1C, for some applications, the pump-outlet tube defines a single axially-facing blood-inlet opening. Alternatively, the pump-outlet tube defines a plurality of lateral blood-inlet openings, e.g., as shown in FIG. 1B.

For some applications, the ventricular assist device is used to assist the functioning of a subject's left ventricle during a percutaneous coronary intervention. In such cases, the ventricular assist device is typically used for a period of up to six hours (e.g., up to ten hours), during a period in which there is risk of developing hemodynamic instability (e.g., during or immediately following the percutaneous coronary intervention). Alternatively or additionally, the ventricular assist device is used to assist the functioning of a subject's left ventricle for a longer period (e.g., 2-20 days, e.g., 4 -14 days) upon a patient suffering from cardiogenic shock, which may include any low-cardiac-output state (e.g., acute myocardial infarction, myocarditis, cardiomyopathy, post-partum, etc.). For some applications, the ventricular assist device is used to assist the functioning of a subject's left ventricle for yet a longer period (e.g., several weeks or months), e.g., in a “bridge to recovery” treatment. For some such applications, the ventricular assist device is permanently or semi-permanently implanted, and the impeller of the ventricular assist device is powered transcutaneously, e.g., using an external antenna that is magnetically coupled to the impeller.

As shown in FIG. 1B, which shows steps in the deployment of the ventricular assist device in the left ventricle, typically the distal end of the ventricular assist device, which comprises pump-outlet tube 24, a distal-tip element 107, and other components described in detail below, is guided to the left ventricle, and inserted into the left ventricle, over a guidewire 10 (e.g., a standard 0.018 inch guidewire), which passes through distal-tip element 107. Typically, guidewire 10 comprises a soft atraumatic distal end. In some embodiments, prior to threading the guidewire through the device, the distal-tip element is straightened using the tip-straightening element described with reference to FIGS. 23A-23C of WO 21/205346 to Tuval, which is incorporated herein by reference.

During the insertion of the distal end of the device into the left ventricle, a delivery catheter 143 is disposed over the distal end of the device, such that delivery catheter 143 holds pump-outlet tube 24 in a radially-constrained configuration. In some embodiments, the delivery catheter is disposed within a standard sheath, such as a 10 Fr sheath. Once the distal end of the device is disposed in the left ventricle (and the sheath, if used, is withdrawn), the pump-outlet tube, along with other components of the device, are removed from the delivery catheter within the left ventricle, by retracting the delivery catheter from over the device. (In this context, advancing the device without advancing the delivery catheter is also referred to as retraction of the delivery catheter.) The retraction of the delivery catheter typically causes self-expandable components of the distal end of the device, such as the pump-outlet tube, to assume non-radially-constrained configurations, as described in further detail hereinbelow. Subsequently, the delivery catheter is typically retracted to the descending aorta, and guidewire 10 is withdrawn from the subject's body.

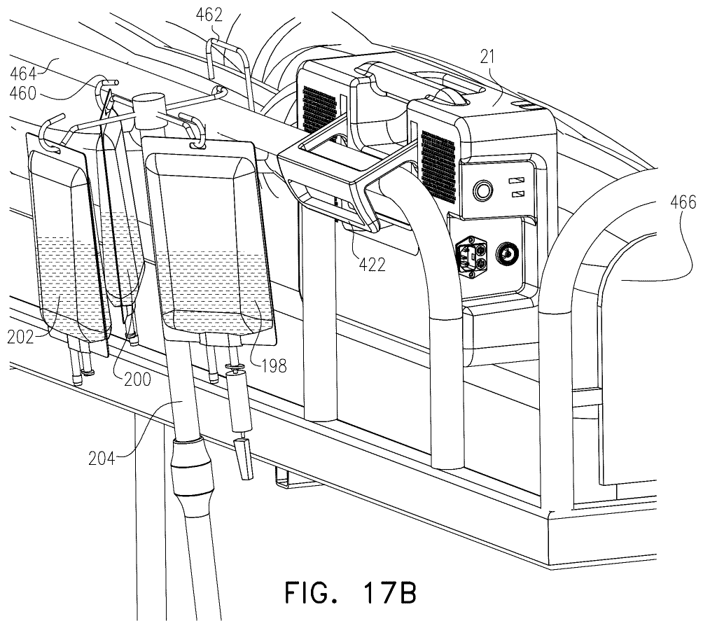

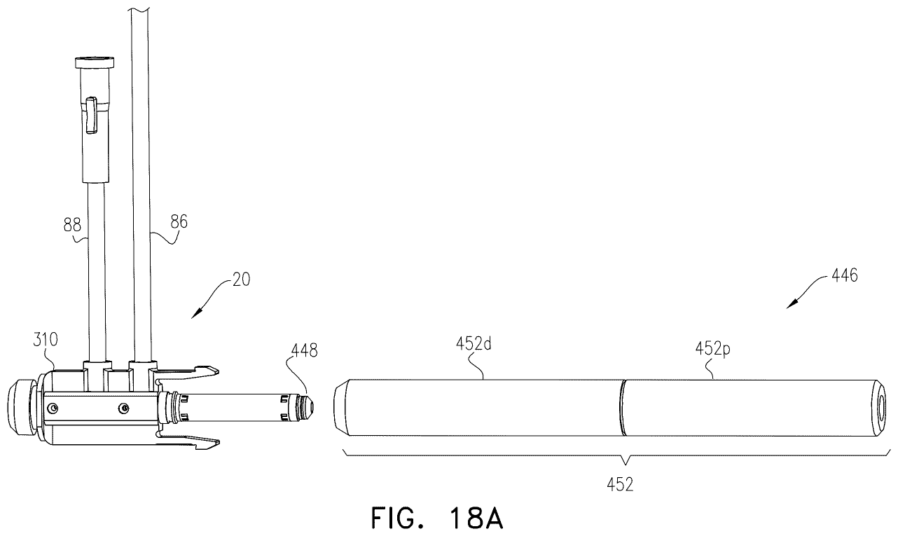

Typically, distal-tip element 107 is positioned at the apex of the left ventricle, e.g., as described with reference to FIGS. 17B-17D of WO 24/057252 to Tuval, which is incorporated herein by reference.

In some embodiments, the positioning of the distal-tip element, along with the withdrawing of the guidewire from the distal-tip element, are performed after the removal of the pump-outlet tube from the delivery catheter. The distal end of the device, when not radially constrained, has greater flexibility, relative to when radially constrained, and this flexibility facilitates positioning the distal-tip element.

In some embodiments, the distal-tip element is positioned at the apex by pushing the distal-tip element toward the apex while withdrawing the guidewire from the distal-tip element. If, on the other hand, the guidewire were to be withdrawn before pushing the distal-tip element toward the apex, it might be challenging to position the distal-tip element correctly. Likewise, if the guidewire were withdrawn after pushing the distal-tip element toward the apex, the withdrawal of the guidewire might cause the distal-tip element to become reshaped, which might also result in incorrect positioning.

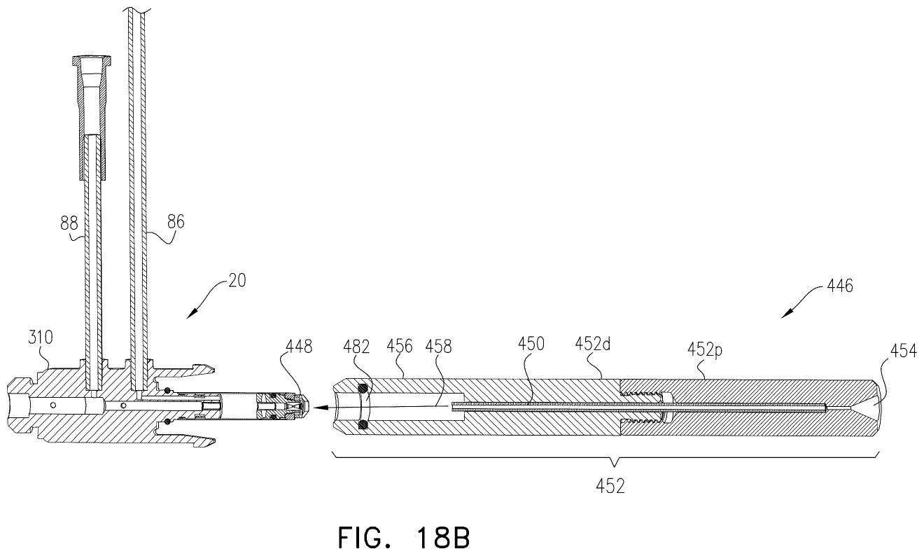

Following the removal of the guidewire from the device, driven-magnet unit 310 of the device (shown in FIGS. 18A-18B, for example) and drive cable 130 are coupled to motor unit 23, and the device is activated.

For some applications, in order to withdraw the left ventricular device from the subject's body at the end of the treatment, the delivery catheter is advanced over the distal end of the device, which causes the self-expandable components of the distal end of the device (e.g., the pump-outlet tube) to assume radially-constrained configurations. Alternatively or additionally, the distal end of the device is retracted into the delivery catheter which causes the self-expandable components of the distal end of the device to assume radially-constrained configurations.

In some embodiments, the distal end of the device is reinserted into the delivery catheter within the descending aorta of the subject. An advantage of performing the reinsertion in the descending aorta—rather than, for example, the left ventricle, ascending aorta, or aortic arch—is that the descending aorta is straight. Moreover, in some cases, the device might release thrombi or debris as the device is reinserted. In the descending aorta, there is less risk of this thrombi or debris reaching the brain.

For some applications (not shown), the ventricular assist device and/or delivery catheter 143 includes an ultrasound transducer at its distal end and the ventricular assist device is advanced toward the subject's ventricle under ultrasound guidance.

System 12 further comprises a control console 21, which comprises a computer processor 25 configured to drive the impeller to rotate. For example, via a cable 224, the computer processor may control motor 15, which, as described above, drives the impeller to rotate via drive cable 130. For some applications, the computer processor is configured to detect or estimate a physiological parameter of the subject (such as left-ventricular pressure, native cardiac output, cardiac afterload, rate of change of left-ventricular pressure, etc.) and to control rotation of the impeller in response thereto. Typically, the operations described herein that are performed by the computer processor, transform the physical state of a memory, which is a real physical article that is in communication with the computer processor, to have a different magnetic polarity, electrical charge, or the like, depending on the technology of the memory that is used. Computer processor 25 is typically a hardware device programmed with computer program instructions to produce a special-purpose computer. For example, when programmed to perform the techniques described herein, computer processor 25 typically acts as a special-purpose, ventricular-assist computer processor and/or a special-purpose, blood-pump computer processor.

For some applications, a purging system 17 (shown in FIG. 1A) drives a fluid (e.g., a glucose solution) to pass through portions of ventricular assist device 20, for example, in order to cool portions of the device, to purge and/or lubricate interfaces between rotating parts and stationary bearings, and/or in order to wash debris from portions of the device.

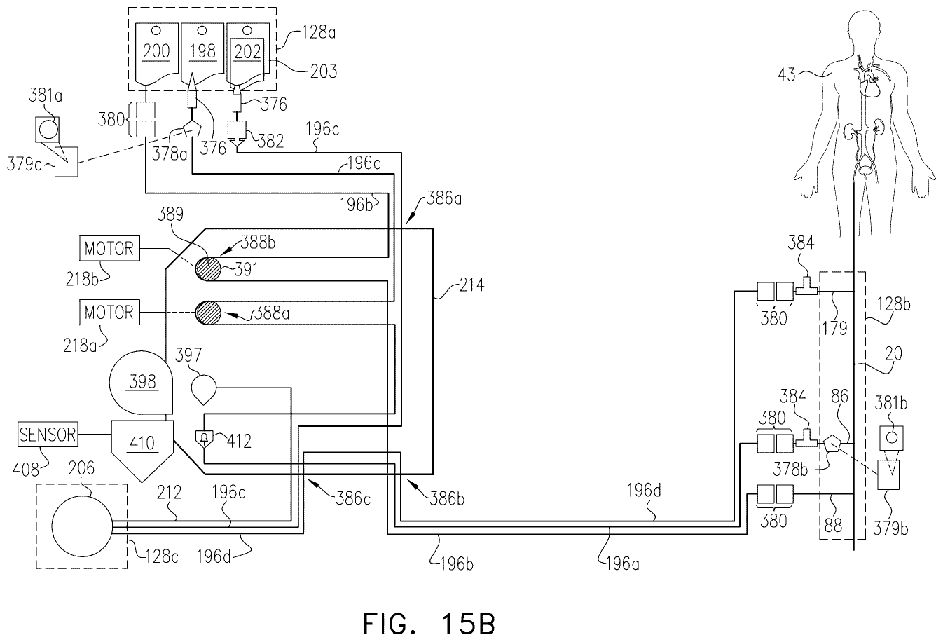

Typically, console 21 further comprises a display 228, embodiments of which are described below with reference to FIG. 14.

Typically, along distal section 102 of pump-outlet tube 24, a frame 34 is disposed at least partly within the pump-outlet tube and around impeller 50, distal-tip element 107 being disposed distally with respect to frame 34. The frame is typically made of a shape-memory alloy, such as nitinol. For some applications, the shape-memory alloy of the frame is shape set such that at least a portion of the frame (and thereby distal section 102 of tube 24) assumes a generally circular, elliptical, or polygonal cross-sectional shape in the absence of any forces being applied to distal section 102 of tube 24. By assuming its generally circular, elliptical, or polygonal cross-sectional shape, the frame is configured to hold the distal section of the pump-outlet tube in an open state. Typically, during operation of the ventricular assist device, the distal section of the pump-outlet tube is configured to be placed within the subject's body such that the distal section of the pump-outlet tube is disposed at least partially within the left ventricle.

For some applications, along proximal section 106 of pump-outlet tube 24, the frame is not disposed within the pump-outlet tube, and the pump-outlet tube is therefore not supported in an open state by frame 34. Pump-outlet tube 24 is typically made of a blood-impermeable collapsible material, such that the pump-outlet tube is collapsible. For example, pump-outlet tube 24 may include a polyurethane, polyester, and/or silicone. Alternatively or additionally, the pump-outlet tube is made of polyethylene terephthalate (PET) and/or polyether block amide (e.g., PEBAX®). For some applications (not shown), the pump-outlet tube is reinforced with a reinforcement structure, e.g., a braided reinforcement structure, such as a braided nitinol tube. Typically, the proximal section of the pump-outlet tube is configured to be placed such that it is at least partially disposed within the subject's ascending aorta. For some applications, the proximal section of the pump-outlet tube traverses the subject's aortic valve, passing from the subject's left ventricle into the subject's ascending aorta, as shown in FIG. 1B.

As described hereinabove, the pump-outlet tube typically defines one or more blood-inlet openings 108 at the distal end of the pump-outlet tube, via which blood flows into the pump-outlet tube from the left ventricle, during operation of the impeller. For some applications, the proximal section of the pump-outlet tube defines one or more blood-outlet openings 109, via which blood flows from the pump-outlet tube into the ascending aorta during operation of the impeller. Typically, the pump-outlet tube defines a plurality of blood-outlet openings 109, for example, between two and eight blood-outlet openings (e.g., between two and four blood-outlet openings). During operation of the impeller, the pressure of the blood flow through the pump-outlet tube typically maintains the proximal section of the tube in an open state. For some applications, in the event that, for example, the impeller malfunctions, the proximal section of the pump-outlet tube is configured to collapse inwardly, in response to pressure outside of the proximal section of the pump-outlet tube exceeding pressure inside the proximal section of the pump-outlet tube. In this manner, the proximal section of the pump-outlet tube acts as a safety valve, preventing retrograde blood flow into the left ventricle from the aorta.

Referring again to FIG. 1C, for some applications, frame 34 is shaped such that the frame defines a proximal conical (or “frustoconical”) portion 36, a central cylindrical portion 38, and a distal conical portion 40. Typically, the proximal conical portion is proximally-facing, i.e., facing such that the narrow end of the cone is proximal with respect to the wide end of the cone. Further typically, the distal conical portion is distally-facing, i.e., facing such that the narrow end of the cone is distal with respect to the wide end of the cone.

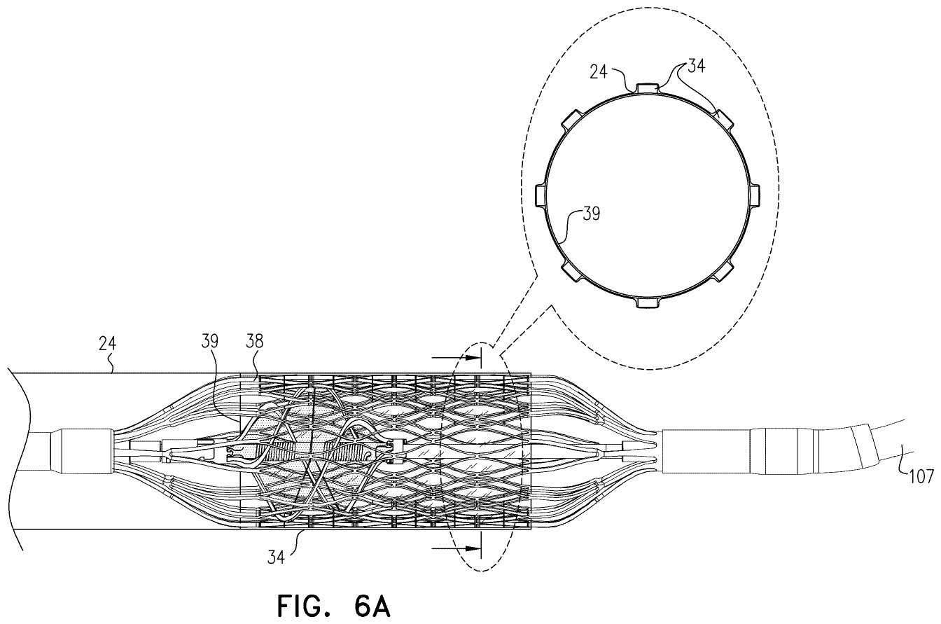

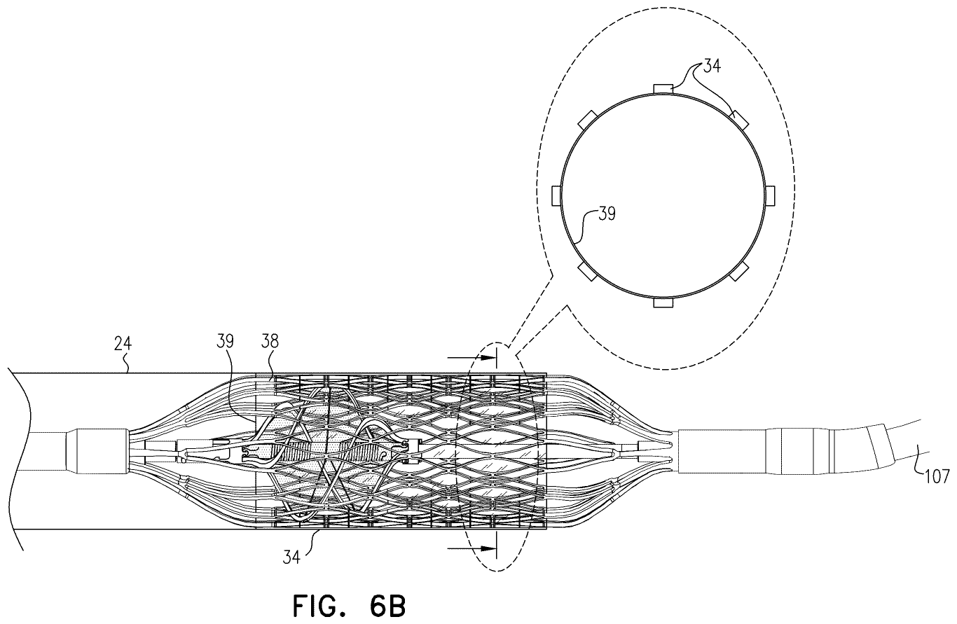

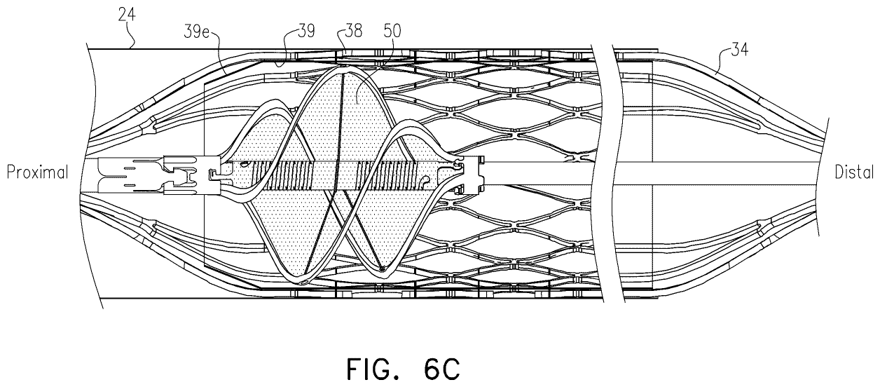

For some applications, within at least a portion of frame 34 (e.g., along all of, or a portion of, the central cylindrical portion of the frame), an inner lining 39, shown in FIG. 1D for example, lines the frame. In accordance with respective applications, inner lining 39 partially overlaps or fully overlaps pump-outlet tube 24 over the portion of the frame that the inner lining lines, as described in further detail hereinbelow with reference to FIGS. 6A-6B. For other applications, as shown in FIG. 1C, the pump-head portion does not comprise inner lining 39.

In some embodiments, pump-outlet tube 24 includes a conical proximal portion 42 and a cylindrical central portion 44, which typically spans proximal section 106 and distal section 102. The proximal conical portion is typically proximally-facing, i.e., facing such that the narrow end of the cone is proximal with respect to the wide end of the cone. Typically, blood-outlet openings 109 are defined by pump-outlet tube 24 such that the openings extend at least partially along the proximal conical portion of tube 24. For some such applications, the blood-outlet openings are teardrop-shaped, as shown in FIG. 1C. Typically, the teardrop-shaped nature of the blood-outlet openings in combination with the openings extending at least partially along the proximal conical portion of tube 24 causes blood to flow out of the blood-outlet openings along flow lines that are substantially parallel with the longitudinal axis of tube 24 at the location of the blood-outlet openings.

For some applications (not shown), the diameter of pump-outlet tube 24 changes along the length of the central portion of the pump-outlet tube, such that the central portion of the pump-outlet tube has a frustoconical shape. For example, the central portion of the pump-outlet tube may widen from its proximal end to its distal end, or may narrow from its proximal end to its distal end. For some applications, at its proximal end, the central portion of the pump-outlet tube has a diameter of between 5 and 7 mm, and at its distal end, the central portion of the pump-outlet tube has a diameter of between 8 and 12 mm.

In some embodiments, drive cable 130 is coupled to an axial shaft 92, which passes through impeller 50 and is configured to rotate the impeller. In some such embodiments, distal-tip element 107 comprises an axial-shaft-receiving tube 126 and a distal-tip portion 120. Axial-shaft-receiving tube 126 is configured to receive a distal portion of axial shaft 92 during axial back-and-forth motion of the axial shaft, and/or during delivery of the ventricular assist device. (Typically, during delivery of the ventricular assist device, the frame is maintained in a radially-constrained configuration, which typically causes the axial shaft to be disposed in a different position with respect to the frame relative to its disposition with respect to the frame during operation of the ventricular assist device.) Typically, distal-tip portion 120 is configured to assume a curved shape upon being deployed within the subject's left ventricle, e.g., as shown in FIG. 1C. For some applications, the curvature of the distal-tip portion is configured to provide an atraumatic tip to ventricular assist device 20. Alternatively or additionally, the distal-tip portion is configured to space blood-inlet openings 108 of the ventricular assist device from walls of the left ventricle.

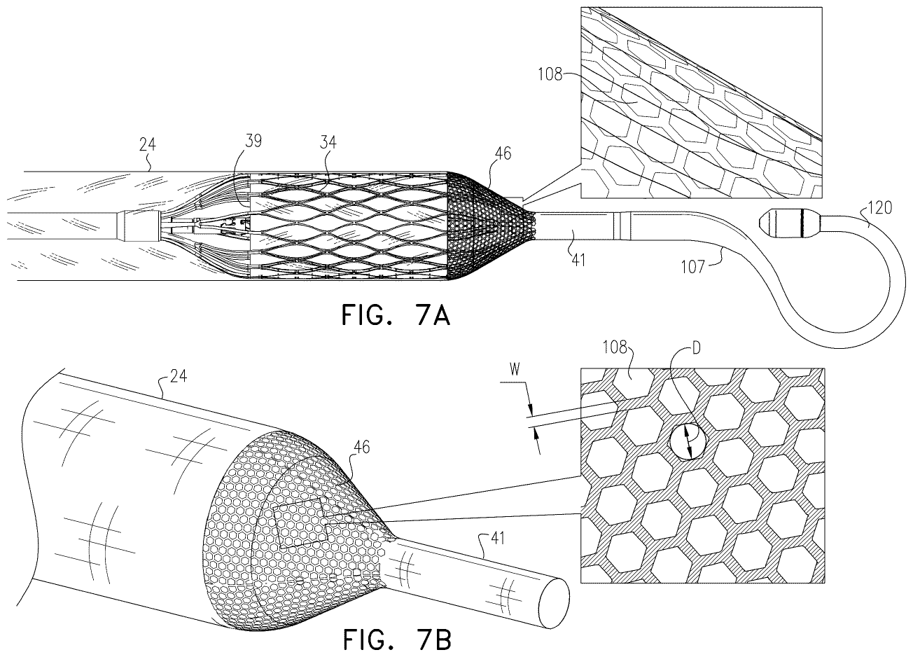

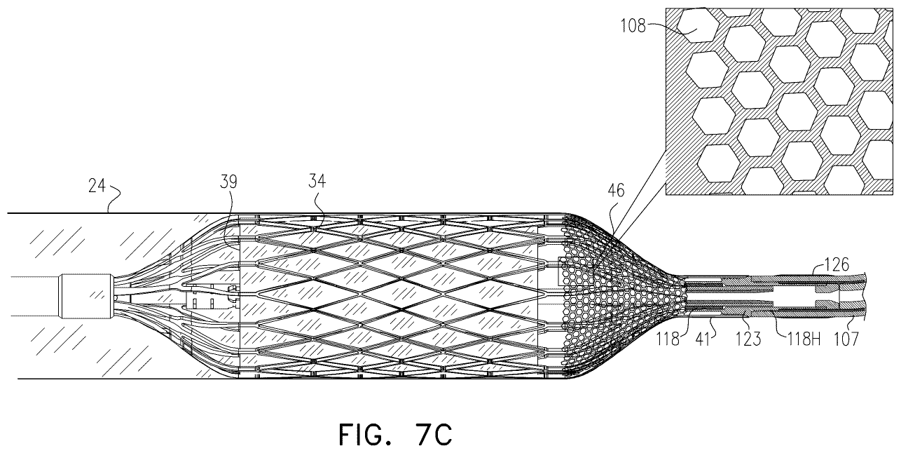

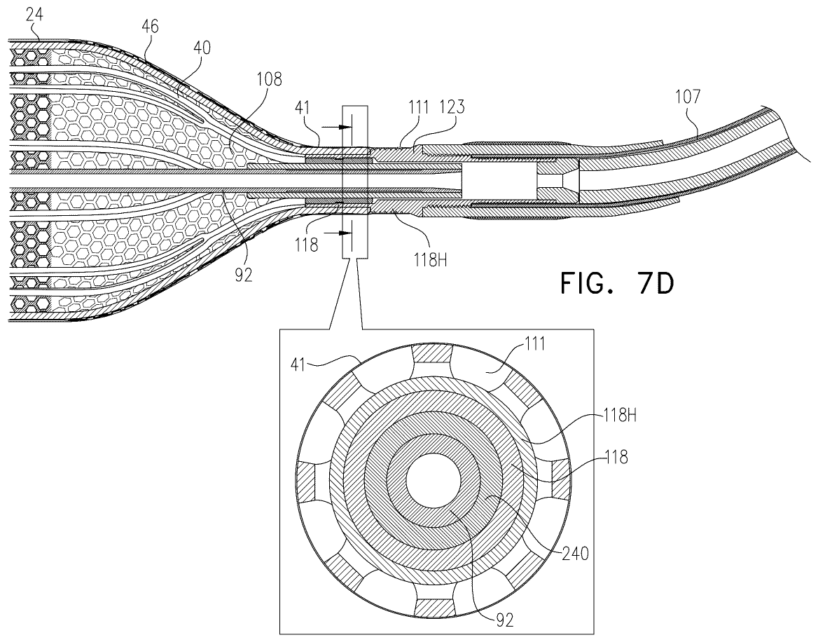

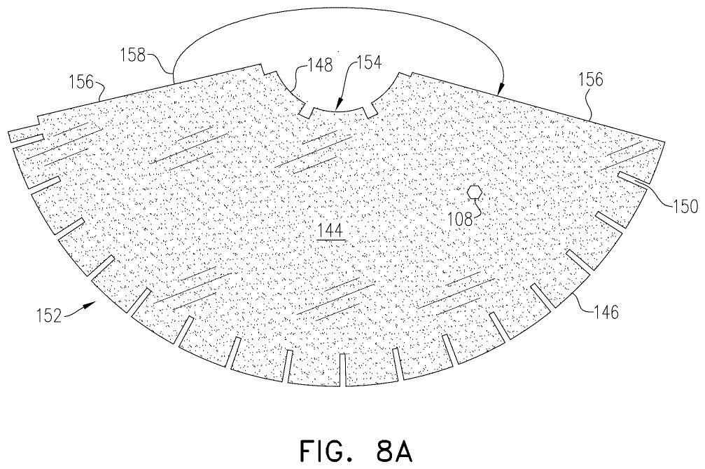

As shown in the enlarged portion of FIG. 1B, for some applications, pump-outlet tube 24 extends to the end of distal conical portion 40 of the frame, and the pump-outlet tube defines a plurality of lateral blood-inlet openings 108, as described in further detail hereinbelow. For some such applications, the pump-outlet tube defines a distal conical (or “frustoconical”) portion 47 that is distally facing, i.e., facing such that the narrow end of the cone is distal with respect to the wide end of the cone. For some such applications (not shown), the pump-outlet tube defines two to four lateral blood-inlet openings (e.g., four lateral blood-inlet openings). Typically, for such applications, each of the blood-inlet openings defines an area of more than 20 square mm (e.g., more than 30 square mm), and/or less than 60 square mm (e.g., less than 50 square mm), e.g., 20-60square mm, or 30-50 square mm. Alternatively or additionally, the outlet tube defines a greater number of smaller lateral blood-inlet openings, e.g., more than 10 blood-inlet openings, more than 100 blood-inlet openings, more than 200 blood-inlet openings, or more than 300 blood-inlet openings, e.g., 50-100 blood-inlet openings, 100-300 blood-inlet openings, or 300-500 blood-inlet openings. For some such applications, each of the blood-inlet openings defines an area of more than 0.05 square mm (e.g., more than 0.1 square mm), and/or less than 3 square mm (e.g., less than 1 square mm), e.g., 0.05-3 square mm, or 0.1-1 square mm. Alternatively, each of the blood-inlet openings defines an area of more than 0.1 square mm (e.g., more than 0.3 square mm), and/or less than 5 square mm (e.g., less than 1 square mm), e.g., 0.1-5 square mm, or 0.3-1 square mm. Such applications are described in further detail hereinbelow, for example, with reference to FIGS. 7A-7D.

As described above, blood-inlet openings 108 are, in some embodiments, defined by distal conical portion 47 of the pump-outlet tube. As such, even the blood-inlet openings that are described as “lateral blood-inlet openings” are not necessarily oriented entirely laterally with respect to the longitudinal axis of the pump-outlet tube. Rather, they are, in some embodiments, obliquely disposed with respect to the longitudinal axis of the pump-outlet tube. By contrast, in some embodiments, the blood-outlet openings are described as “laterally-facing blood-outlet openings” because in such embodiments the blood-outlet openings are disposed laterally with respect to the longitudinal axis of the pump-outlet tube, by virtue of being defined by the central cylindrical portion of the pump-outlet tube. (In other embodiments, the blood-outlet openings are disposed obliquely with respect to the longitudinal axis of the pump-outlet tube, by virtue of being defined at least partially by the proximal conical portion of the pump-outlet tube.)

In some embodiments, proximally to proximal conical portion 42, the pump-outlet tube defines a tubular coupling portion 45, via which the pump-outlet tube is coupled (e.g., via an adhesive) to delivery tube 142. For some such embodiments, the pump-outlet tube is manufactured from a single continuous tube, with respective portions of the tube being molded to define tubular coupling portion 45, proximal conical portion 42, distal conical portion 47, and cylindrical central portion 44. Typically, in such cases, the blood-inlet openings and the blood-outlet openings are cut (e.g., laser cut) from the tube. In some embodiments, before adhering the tubular coupling portion to delivery tube 142 of the ventricular assist device, the tubular coupling portion is cut (e.g., in a tapered manner), so as to reduce the thickness of the layer of the pump-outlet tube that is coupled to delivery tube 142 and/or to prevent folds forming in the tubular coupling portion of the pump-outlet tube.

In some embodiments, (a) blood-outlet openings 109 are defined by portions of the wall of the blood outlet tube that at least partially extends into the proximal conical portion of the pump-outlet tube, and/or (b) blood-outlet openings 109 are laterally facing, by virtue of being defined by the central cylindrical portion of pump-outlet tube 24. The scope of the present disclosure includes combining other features of the pump-outlet tube and/or other portions of the ventricular assist device with any configuration of blood-outlet openings that are described and/or shown in the present application.

It is noted that the above description of pump-outlet tube 24 and blood-outlet openings 109 is also applicable to other embodiments described herein. Furthermore, the scope of the present disclosure includes combining a pump-outlet-tube that defines a single axially-facing blood-inlet opening 108 as shown in FIG. 1C, or a pump-outlet-tube that defines a plurality of lateral blood-inlet openings 108 as shown in FIG. 1B, with other features of the ventricular assist device that are described herein, mutatis mutandis.

In some embodiments, a pressure sensor 216 measures the pressure of blood in the subject's left ventricle, e.g., as described in WO 24/057252 to Tuval, which is incorporated herein by reference.

Reference is now made to FIG. 1D, which is a schematic illustration of a pump-head portion of a ventricular assist device, in accordance with some embodiments.

In some embodiments, pump-outlet tube 24 does not define a tubular coupling portion. Rather, initially, the proximal portion of the tube that will form the proximal conical section is shaped as a cylinder (which is typically continuous with the cylinder shape of the central portion). From this proximal portion of the tube, strips are cut (e.g., laser cut), leaving other strips 29 still attached to, and extending proximally from, the central cylindrical portion of the tube. The proximal ends of strips 29 are then adhered to delivery tube 142 of the ventricular assist device, in such a manner that they define a proximal conical portion of the pump-outlet tube that defines blood-outlet openings 109. In other words, blood-outlet openings 109 are formed between strips 29, by adhering the strips to delivery tube 142 of the ventricular assist device.

For some applications, by forming the proximal conical portion of the pump-outlet tube and the blood-outlet openings using the latter method, the thickness of the layer of the pump-outlet tube that is coupled to delivery tube 142 is less than the thickness of the tubular coupling portion as formed by the former method. For some applications, this reduces the sharpness of the diameter change at the interface between delivery tube 142 and the region at which the proximal end of the pump-outlet tube is coupled to the delivery tube.

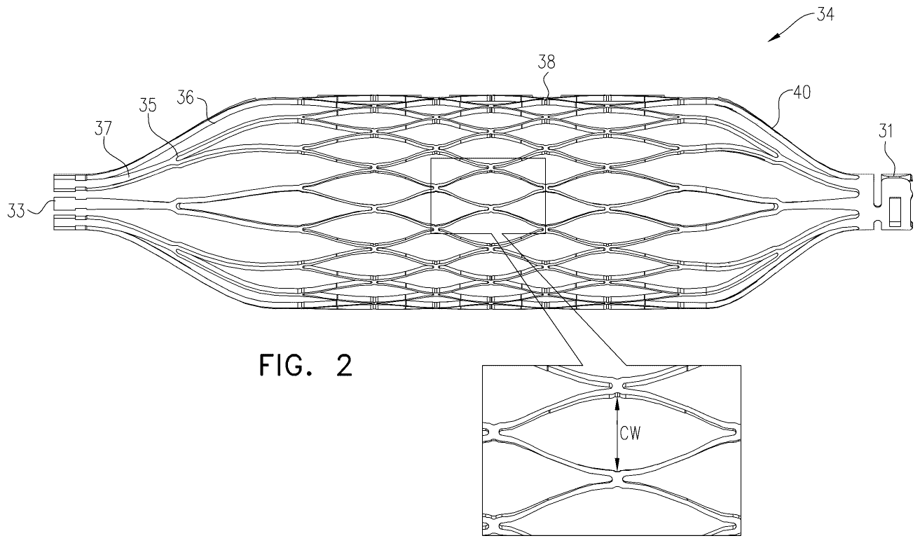

Reference is now made to FIG. 2, which is schematic illustration of frame 34 that houses an impeller of ventricular assist device 20, in accordance with some embodiments. Frame 34 is typically made of a shape-memory alloy, such as nitinol, and the shape-memory alloy of the frame is shape set such that the central portion 38 of the frame (and thereby tube 24) assumes a generally circular, elliptical, or polygonal cross-sectional shape in the absence of any forces being applied to pump-outlet tube 24. By assuming its generally circular, elliptical, or polygonal cross-sectional shape, the frame is configured to hold the distal portion of the tube in an open state. (Given that, typically, central portion 38 of the frame has a circular cross-section, the central portion of the frame is also referred to herein as the “cylindrical portion” of the frame.)

Typically, the frame is a stent-like frame, in that it comprises struts 37 that, in turn, define cells. In some embodiments, the frame is laser cut from a metal or alloy tube. Typically, the frame is covered with pump-outlet tube 24, and/or covered with an inner lining 39, described hereinbelow with reference to FIGS. 6A-6B. As described hereinbelow, for some applications, impeller 50 undergoes axial back-and-forth motion with respect to frame 34. Typically, over the course of the motion of the impeller with respect to the frame, the location of the portion of the impeller that defines the maximum span of the impeller is disposed within central cylindrical portion 38 of frame 34. In some cases, if the cells of the central cylindrical portion 38 of frame 34 are too large, then pump-outlet tube 24, and/or inner lining 39 (FIG. 1D), gets stretched between edges of the cells, such that the pump-outlet tube 24, and/or inner lining 39, does not define a circular cross-section. For some applications, if this occurs in the region in which the portion of the impeller that defines the maximum span of the impeller is disposed, this results in a substantially non-constant gap between the edges of the impeller blades and tube 24 (and/or inner lining) at that location, over the course of a rotation cycle of the impeller. For some applications, this may lead to increased hemolysis relative to if there were a substantially constant gap between the edges of the impeller blades and tube 24 (and/or inner lining) at that location, over the course of the rotation cycle of the impeller.

Referring to FIG. 2, at least partially in view of the issues described in the above paragraph, within central cylindrical portion 38 of frame 34, the frame defines a large number of relatively small cells. Typically, when the frame is disposed in its non-radially-constrained configuration, the maximum cell width CW of the each of the cells (i.e., the distance from the inner edge of the strut at the central junction on one side of the cell to the inner edge of the strut at the central junction on the other side of the cell, as measured around the circumference of cylindrical portion 38) within the cylindrical portion of the frame is less than 2 mm, e.g., between 1.4 mm and 1.6 mm, or between 1.6 and 1.8 mm. Since the cells are relatively small, inner lining 39 defines a substantially circular cross-section within the cylindrical portion of the frame.

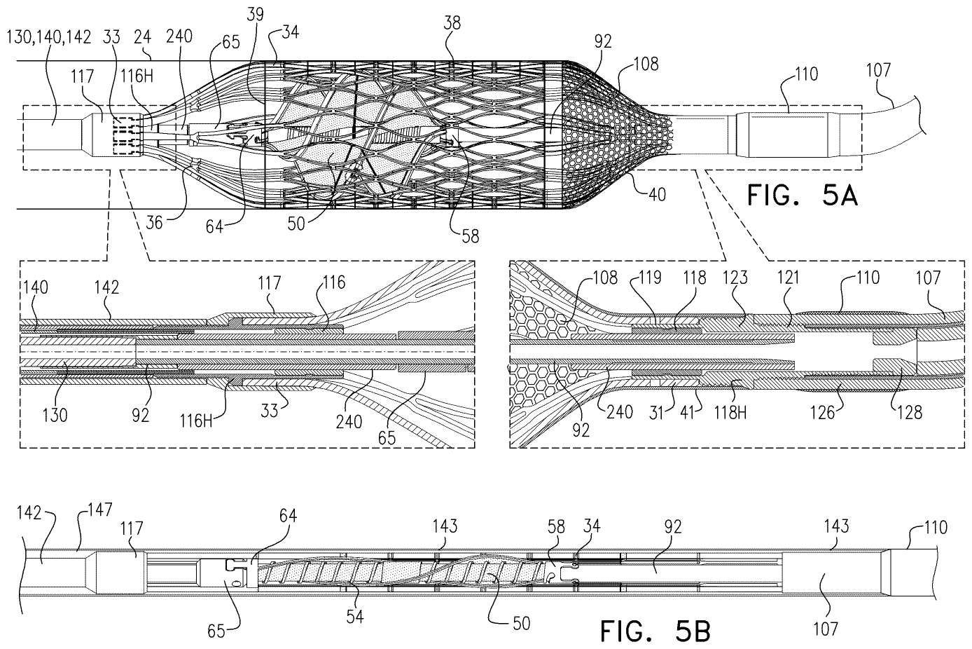

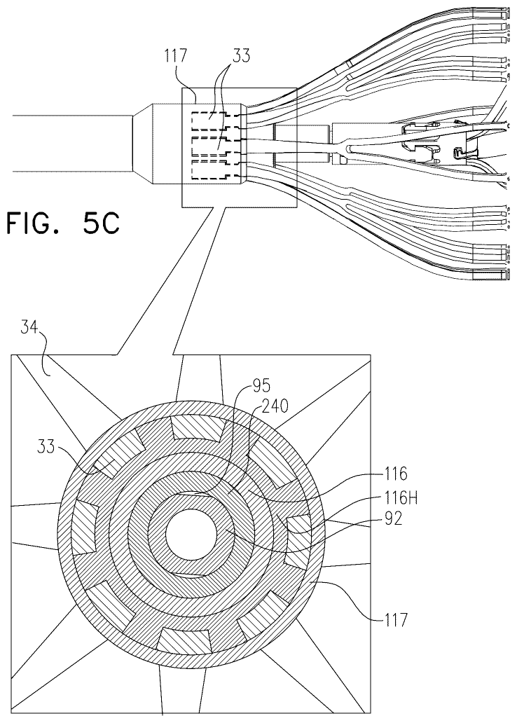

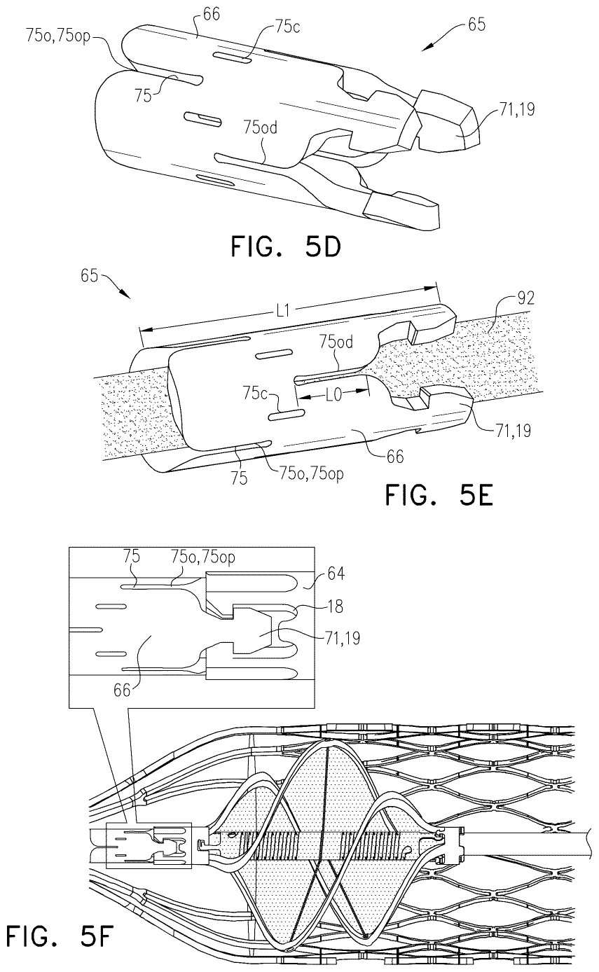

Still referring to FIG. 2, and starting from the distal end of the frame (which is to the right of the figure), typically the frame defines the following portions: (a) coupling portion 31 via which the frame is coupled to a distal bearing housing 118H (shown in FIG. 5A) of the ventricular assist device, (b) distal conical portion 40, (c) central cylindrical portion 38, (d) proximal conical portion 36, and (e) proximal strut junctions 33. As illustrated, as the frame transitions from a proximal end of the frame toward the center of the frame (e.g., as the frame transitions from proximal strut junctions 33, through proximal conical portion 36, and to central cylindrical portion 38), struts 37 of the frame pass through junctions 35, at which the two struts branch from a single strut, in a Y-shape.

During the assembly of the ventricular assist device, the impeller is inserted into frame 34, typically via the open proximal end of the frame. In some embodiments, prior to the insertion of the impeller, pump-outlet tube 24 (FIG. 1C) is fixed over the frame, including over the distal end of the frame. In such embodiments, the impeller cannot be inserted via the distal end of the frame, since the distal end of the frame is covered by pump-outlet tube 24. Therefore, proximal strut junctions 33 are maintained in open states, in order for the impeller to be placed within the frame via the proximal end of the frame. Subsequently to the impeller being inserted via the proximal end of the frame, the proximal strut junctions are closed. For some applications, the proximal strut junctions are closed around the outside of a proximal bearing housing 116H (shown in FIG. 5A), as described in further detail hereinbelow with reference to FIGS. 5A-5B. Typically, a securing element 117 (e.g., a ring shown in FIG. 5A) holds the strut junctions in their closed configurations around the outside of proximal bearing housing 116H.

In other embodiments, the pump-outlet tube does not extend to the distal end of frame 34, or the distal portion of the pump-outlet tube is fixed over the distal portion of the frame only after the impeller has been inserted into the frame. In some such embodiments, the impeller is inserted into the frame via the distal end of the frame.

In some embodiments, prior to or subsequently to the insertion of the impeller, distal coupling portion 31 is coupled to a distal bearing housing 118H (shown in FIG. 5A), e.g., via a snap-fit mechanism.

Typically, when disposed in its non-radially constrained configuration, frame 34 has a total length of more than 25 mm (e.g., more than 30 mm), and/or less than 50 mm (e.g., less than 45 mm), e.g., 25-50 mm, or 30-45 mm. Typically, when disposed in its radially-constrained configuration (within delivery catheter 143), the length of the frame increases by between 2 and 5 mm. Typically, when disposed in its non-radially constrained configuration, the central cylindrical portion of frame 34 has a length of more than 12 mm (e.g., more than 15 mm), and/or less than 28 mm (e.g., less than 24 mm), e.g., 12-28 mm, or 15-24 mm. For some applications, a ratio of the length of the central cylindrical portion of the frame to the total length of the frame is more than 1:3 and/or less than 3:4, e.g., between 1:3 and 3:4.

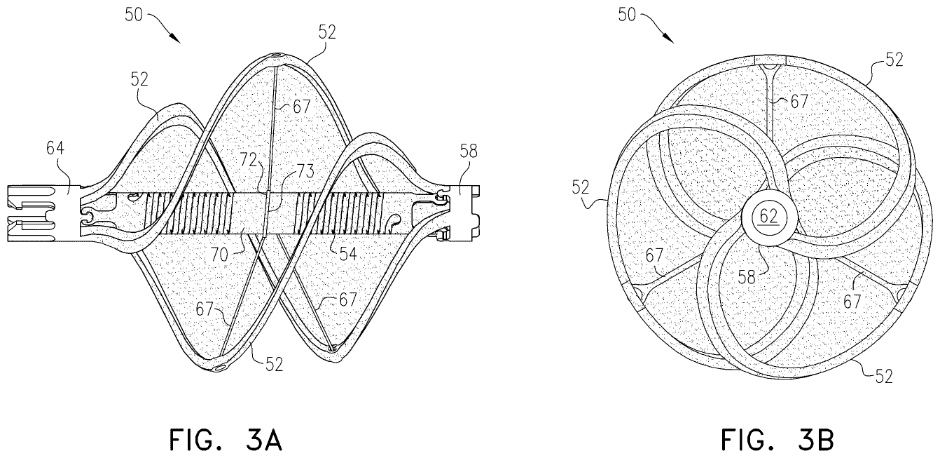

Reference is now made to FIGS. 3A-3B, which are schematic illustrations of impeller 50, in accordance with some embodiments. Typically, the impeller includes at least one outer helical elongate element 52, which winds around a central axial spring 54, such that the helix defined by the helical elongate element is coaxial with the central axial spring. Typically, the impeller includes two or more helical elongate elements (e.g., three helical elongate elements, as shown in FIGS. 3A-3B). For some applications, the helical elongate elements and the central axial spring are made of a shape-memory material, e.g., a shape-memory alloy, such as nitinol. Typically, each of the helical elongate elements and the central axial spring support a film 56 of a material (e.g., an elastomer, such as a polyurethane, and/or silicone) therebetween. For some applications, the film of material includes pieces of nitinol embedded therein, for example in order to strengthen the film of material.

Each of the helical elongate elements, together with the film extending from the helical elongate element to the spring, defines a respective impeller blade, with the helical elongate elements defining the outer edges of the blades, and the axial spring defining the axis of the impeller. Typically, the film of material extends along and coats the spring.

Typically, proximal ends of spring 54 and helical elongate elements 52 extend from a proximal bushing (i.e., sleeve bearing) 64 of the impeller, such that the proximal ends of spring 54 and helical elongate elements 52 are disposed at a similar radial distance from the longitudinal axis of the impeller, as each other. Similarly, typically, distal ends of spring 54 and helical elongate elements 52 extend from a distal bushing 58 of the impeller, such that the distal ends of spring 54 and helical elongate elements 52 are disposed at a similar radial distance from the longitudinal axis of the impeller, as each other. The helical elongate elements typically rise gradually from the proximal bushing before reaching a maximum span and then falling gradually toward the distal bushing. Typically, the helical elongate elements are symmetrical along their lengths, such that the rising portions of their lengths are symmetrical with respect to the falling portions of their lengths. Typically, the impeller defines a lumen 62 therethrough, with the lumen typically extending through, and being defined by, spring 54, as well as proximal bushing 64 and distal bushing 58, of the impeller.

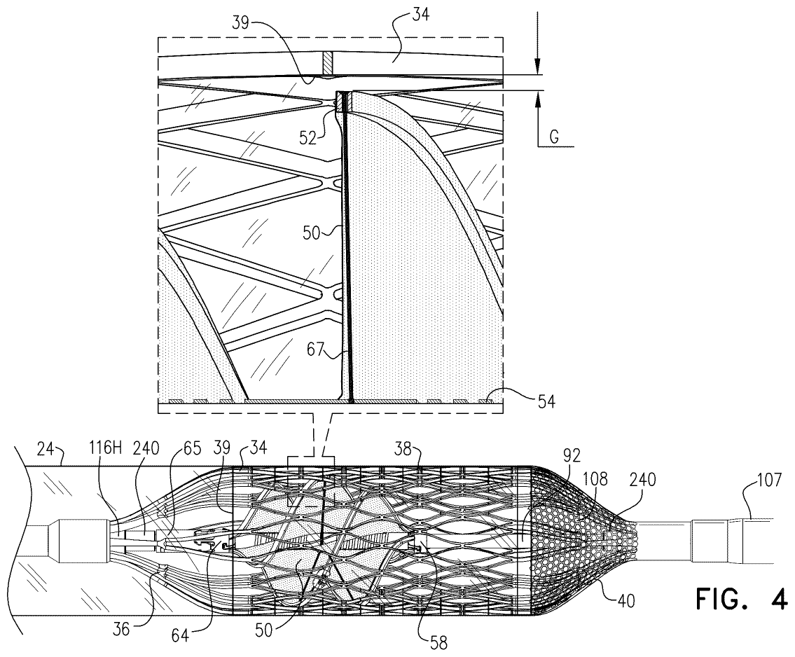

Reference is now made to FIG. 4, which is a schematic illustration of impeller 50 disposed inside frame 34 of ventricular assist device 20, in accordance with some embodiments. For some applications, within at least a portion of frame 34 (e.g., along all of, or a portion of, central cylindrical portion 38 of the frame), inner lining 39 lines the frame. In accordance with respective applications, the inner lining partially overlaps or fully overlaps with pump-outlet tube 24 over the portion of the frame that the inner lining lines, as described in further detail hereinbelow with reference to FIGS. 6A-6B.

As shown in FIG. 4, typically there is a gap G between the outer edge of impeller 50 and inner lining 39, even at a location at which the span of the impeller is at its maximum. For some applications, it is desirable that the gap between the outer edge of the blade of the impeller and inner lining 39 be relatively small, in order for the impeller to efficiently pump blood from the subject's left ventricle into the subject's aorta. (It is noted that, by virtue of the relatively small gap between the outer edge of impeller 50 and inner lining 39 even at a location at which the span of the impeller is at its maximum, as well as the shape of the impeller, the impeller functions as an axial-flow impeller, with the impeller pumping blood in the axial direction from a distal end of pump-outlet tube 24 to the proximal end of the pump-outlet tube.) It is also desirable that a gap between the outer edge of the blade of the impeller and the inner surface of frame 34 be maintained throughout the rotation of the impeller within frame 34, for example, in order to reduce the risk of hemolysis.

For some applications, when impeller 50 and frame 34 are both disposed in non-radially-constrained configurations and prior to operation of the impeller, gap G between the outer edge of the impeller and the inner lining 39, at the location at which the span of the impeller is at its maximum, is greater than 0.05 mm (e.g., greater than 0.1 mm), and/or less than 1 mm (e.g., less than 0.4 mm), e.g., 0.05-1 mm, or 0.1-0.4 mm.

Typically, an axial shaft 92 passes through the axis of impeller 50, via lumen 62 of the impeller. For some applications, the axial shaft is rigid, e.g., a rigid tube. For some applications, the axial shaft is made of a shape-memory material (e.g., a shape memory alloy, such as nitinol). Typically, such materials have some elasticity, such that in the event that the axial shaft becomes bent (e.g., during delivery of the pump head to the left ventricle), the axial shaft still assumes a straight shape, once deployed inside the subject's body.

Proximal bushing 64 is disposed over axial shaft 92, and distal bushing 58 is disposed over the axial shaft distally from the proximal bushing. For some applications, proximal bushing 64 of the impeller is coupled to the shaft such that the axial position of the proximal bushing with respect to the shaft is fixed, and distal bushing 58 of the impeller is slidable with respect to (i.e., is slidable along) the shaft. For example, the proximal bushing may be coupled to a coupling element 65 disposed on the axial shaft (shown in FIG. 4), for example via a snap-fit mechanism. Alternatively, distal bushing 58 of the impeller is coupled to the shaft such that the axial position of the distal bushing with respect to the shaft is fixed, and proximal bushing 64 of the impeller is slidable with respect to the shaft.

The axial shaft itself is radially stabilized via a proximal radial bearing 116 and a distal radial bearing 118 (FIG. 5A). In turn, the axial shaft, by passing through lumen 62 defined by the impeller, radially stabilizes the impeller with respect to the inner surface of frame 34, such that even a relatively small gap between the outer edge of the blade of the impeller and the inner surface of frame 34 (e.g., a gap that is as described above) is maintained, during rotation of the impeller.

Referring again to FIGS. 3A-3B, for some applications, the impeller includes a plurality of elongate elements 67 extending radially from central axial spring 54 to outer helical elongate elements 52. For some applications, as shown, the impeller includes a single integrated impeller-overexpansion-prevention element 72 that defines a plurality of elongate elements 67. For some applications, impeller-overexpansion-prevention element 72 defines a ring 73 and the plurality of elongate elements 67 extending radially from the ring. For some applications, ring 73 of element 72 is placed around (and coupled to) the spring, e.g., by being placed around a tube 70, which is typically disposed at the longitudinally-central location of the spring. The ends of respective elongate elements 67 are then coupled to respective helical elongate elements 52.

For some applications, elongate elements 67 maintain helical elongate element 52 (which defines the outer edge of the impeller blade) within a given distance with respect to the central axial spring. In this manner, the elongate elements are configured to prevent the outer edge of the impeller from being forced radially outward due to forces exerted upon the impeller during the rotation of the impeller. The elongate elements are thereby configured to maintain the gap between the outer edge of the blade of the impeller and the inner surface of frame 34, during rotation of the impeller.

Elongate elements 67 are typically flexible but are substantially non-stretchable along the axis defined by the elongate elements. Typically, each of elongate elements 67 is configured not to resist compression. Rather, each elongate element 67 is configured to exert a tensile force upon helical elongate element 52 that prevents helical elongate element 52 from moving radially outward, such that (in the absence of elongate element 67) a separation between helical elongate element 52 and central axial spring 54 would be greater than a length of elongate element 67. When a force is acting upon the impeller that would cause the helical elongate element 52 to move radially outward (in the absence of elongate element 67), the impeller-overexpansion-prevention element is configured to prevent radial expansion of the impeller. Typically, a respective elongate element 67 is disposed within each one of the impeller blades and is configured to prevent the impeller blade from radially expanding. For some applications, element 72 is made of polyester, and/or another polymer or a natural material that contains fibers, and/or nitinol (or a similar shape-memory alloy).

Typically, impeller 50 is inserted into the left ventricle transcatheterally, while impeller 50 is in a radially-constrained configuration. In the radially-constrained configuration, both helical elongate elements 52 and central axial spring 54 are axially elongated and radially constrained. Typically, film 56 of the material (e.g., silicone and/or a polyurethane) changes shape to conform to the shape changes of the helical elongate elements and the axial support spring, both of which support the film of material. Typically, using a spring to support the inner edge of the film allows the film to change shape without the film becoming broken or collapsing, due to the spring providing a large surface area to which the inner edge of the film bonds. For some applications, using a spring to support the inner edge of the film reduces a diameter to which the impeller can be radially constrained, relative to if, for example, a rigid shaft were to be used to support the inner edge of the film, since the diameter of the spring itself can be reduced by axially elongating the spring.

As described hereinabove, for some applications, proximal bushing 64 of impeller 50 is coupled to axial shaft 92 such that the axial position of the proximal bushing with respect to the shaft is fixed, and distal bushing 58 of the impeller is slidable with respect to the shaft. For example, the proximal bushing may be coupled to coupling element 65 disposed on the axial shaft (shown in FIG. 4), for example via a snap-fit mechanism. For some applications, when the impeller is radially constrained for the purpose of inserting the impeller into the ventricle or for the purpose of withdrawing the impeller from the subject's body, the impeller axially elongates by the distal bushing sliding along the axial shaft distally. Alternatively (not shown), distal bushing 58 of the impeller is coupled to the shaft such that the axial position of the distal bushing with respect to the shaft is fixed, and proximal bushing 64 of the impeller is slidable with respect to the shaft. For some such applications, when the impeller is radially constrained for the purpose of inserting the impeller into the ventricle or for the purpose of withdrawing the impeller from the subject's body, the impeller axially elongates by the proximal bushing sliding along the axial shaft proximally. Subsequent to being released inside the subject's body, the impeller assumes its non-radially-constrained configuration (in which the impeller is typically disposed during operation of the impeller), which is as shown in FIGS. 3A-3B.

Reference is now made to FIGS. 5A and 5B, which are schematic illustrations of impeller 50 and frame 34 of ventricular assist device 20, respectively in non-radially-constrained and radially-constrained states thereof, in accordance with some embodiments. The impeller and the frame are typically disposed in the radially-constrained states during the transcatheteral insertion of the impeller and the frame into the subject's body, and are disposed in the non-radially-constrained states during operation of the impeller inside the subject's left ventricle. Reference is also made to FIG. 5C, which is an enlarged schematic illustration of the proximal end of the frame of the ventricular assist device, in accordance with some embodiments.

As indicated in FIG. 5B, the frame and the impeller are typically maintained in radially-constrained configurations by delivery catheter 143. Typically, in the radially-constrained configuration of the impeller, the impeller has a total length of more than 15 mm (e.g., more than 20 mm), and/or less than 30 mm (e.g., less than 25 mm), e.g., 15-30 mm, or 20-25 mm. Further typically, in the non-radially constrained configuration of the impeller, the impeller has a length of more than 10 mm (e.g., more than 12 mm), and/or less than 20 mm (e.g., less than 18 mm), e.g., 10-20 mm, or 12-18 mm. Typically, when disposed in its non-radially constrained configuration, frame 34 has a total length of more than 25 mm (e.g., more than 30 mm), and/or less than 50 mm (e.g., less than 45 mm), e.g., 25-50 mm, or 30-45 mm. Typically, when disposed in its radially-constrained configuration (within delivery catheter 143), the length of the frame increases by between 2 and 5 mm.

For some applications, when the impeller is disposed in its non-radially-constrained configurations and prior to operation of the impeller, the outer diameter of the impeller at the location at which the outer diameter of the impeller is at its maximum is more than 7 mm (e.g., more than 8 mm), and/or less than 11 mm (e.g., less than 10 mm), e.g., 7-11 mm, or 8-10 mm. For some applications, when frame 34 is disposed in its non-radially-constrained configuration, the inner diameter of frame 34 (as measured from the inside of inner lining 39 on one side of the frame to the inside of inner lining on the opposite side of the frame) is greater than 7.5 mm (e.g., greater than 8.5 mm), and/or less than 10.5 mm (e.g., less than 9.5 mm), e.g., 7.5-10.5 mm, or 8.5-9.5 mm. For some applications, when the frame is disposed in its non-radially-constrained configuration, the outer diameter of frame 34 is greater than 8 mm (e.g., greater than 9 mm), and/or less than 12 mm (e.g., less than 11 mm), e.g., 8-12 mm, or 9-11 mm.

For some applications, when the impeller is disposed in its radially-constrained configuration, e.g., during delivery of the ventricular assist device via delivery catheter 143, the outer diameter of the impeller at the location at which the outer diameter of the impeller is at its maximum is more than 1.5 mm (e.g., more than 2 mm), and/or less than 3 mm (e.g., less than 2.5 mm), e.g., 1.5-3 mm, or 2-2.5 mm. For some applications, the ratio between the outer diameter of the impeller at the location at which the outer diameter of the impeller is at its maximum in (a) the impeller's non-radially constrained configuration versus (b) the impeller's radially constrained configuration is more than 3:1, e.g., more than 7:2, or more than 4:1.

For some applications, when the frame is disposed in its radially-constrained configuration, e.g., during delivery of the ventricular assist device via delivery catheter 143, the outer diameter of the frame is more than 2 mm (e.g., more than 2.5 mm), and/or less than 4 mm (e.g., less than 3.5 mm), e.g., 2-4 mm, or 2.5-3.5 mm. For some applications, the ratio between the outer diameter of the frame in (a) the frame's non-radially constrained configuration versus (b) the frame's radially constrained configuration is more than 5:2, e.g., more than 3:1.

As described hereinabove, typically, axial shaft 92 passes through the axis of impeller 50, via lumen 62 of the impeller. Typically, proximal bushing 64 of the impeller is coupled to the shaft via a coupling element 65 such that the axial position of the proximal bushing with respect to the shaft is fixed, and distal bushing 58 of the impeller is slidable with respect to the shaft. Alternatively, distal bushing 58 of the impeller is coupled to the shaft such that the axial position of the distal bushing with respect to the shaft is fixed, and proximal bushing 64 of the impeller is slidable with respect to the shaft.

The axial shaft itself is radially stabilized via a proximal radial bearing 116 and a distal radial bearing 118. Typically, proximal bearing housing 116H is disposed around, and houses, the proximal bearing, and distal bearing housing 118H is disposed around, and houses, the distal bearing. For some such applications, the radial bearings and the bearing housings are made of respective, different materials from each other. For example, the radial bearings may be made of a first material that has a relatively high hardness, such as ceramic (e.g., zirconia), and the bearing housings may be made of a second material that is moldable into a desired shape, such as a metal or an alloy (e.g., stainless steel, cobalt chromium, and/or nitinol).

For some applications, axial shaft 92 is made of a metal or an alloy, such as stainless steel. For some such applications, the axial shaft is covered with ceramic sleeves 240 (e.g., zirconia sleeves) along regions of the axial shaft that come into contact with either of the proximal and distal bearings 116, 118 during operation of the ventricular assist device. In this manner, the radial interfaces between the axial shaft and the proximal and distal bearings are ceramic-ceramic interfaces. As described in further detail herein, in some embodiments, the impeller and the axial shaft are configured to undergo axial back-and-forth motion during operation of the ventricular assist device. Therefore, for some applications, at locations along the axial shaft corresponding to each of the proximal and distal bearings, the axial shaft is covered with the ceramic sleeve along a length of more than 5 mm, e.g., more than 7 mm. In this manner, over the course of the axial back-and-forth motion of the axial shaft, the ceramic sleeves remain in contact with the radial bearings.

For some applications, along each portion of the axial shaft that is covered with a ceramic sleeve, the shaft is shaped (e.g., via milling, molding, or a different shaping process) to define one or more grooves or indents 95, as shown in the transverse cross-sectional view of FIG. 5C. Alternatively or additionally (not shown), the inner surface of the ceramic sleeve is shaped to define or more grooves or indents. For some such applications, in order to bond the sleeve to the axial shaft, an adhesive is injected into the groove or indent and the adhesive then spreads from the groove or indent across the interface between the axial shaft and the sleeve.

For some applications, the proximal bearing housing 116H and distal bearing housing 118H perform additional functions. Referring first to the proximal bearing housing, as described hereinabove, for some applications, proximal strut junctions 33 of frame 34 are closed around the outside of the proximal bearing housing. For some applications, the outer surface of the proximal bearing housing defines grooves that are shaped such as to receive the proximal strut junctions. For example, as shown, the proximal strut junctions have widened heads, and the outer surface of the proximal bearing housing defines grooves that are shaped to conform with the widened heads of the proximal strut junctions. Typically, securing element 117 (which typically includes a ring) holds the strut junctions in their closed configurations around the outside of proximal bearing housing 116H.

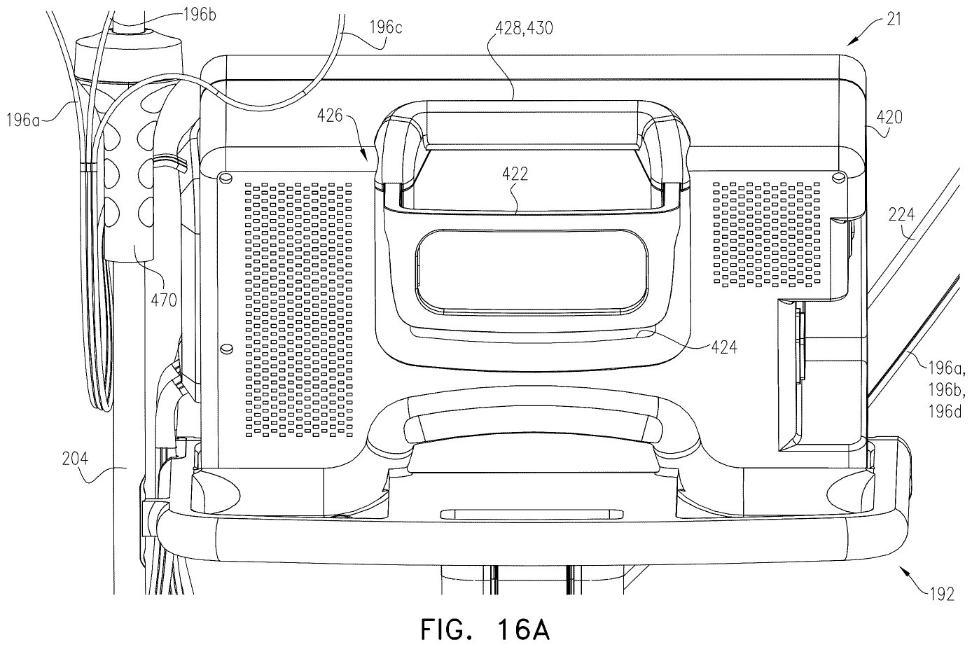

For some applications, additional portions of the ventricular assist device are coupled to the proximal bearing housing. For example, for some applications, drive cable 130 extends from outside the subject's body to axial shaft 92, and is coupled to the axial shaft such that the axial shaft rotates with the drive cable. Typically, the drive cable rotates within a first outer tube 140, which functions as a drive-cable-bearing tube, and which extends from outside the subject's body to the proximal bearing housing. For some applications, the first outer tube is disposed within a second outer tube 142 (also referred to herein as a “delivery tube”), which also extends from outside the subject's body to the proximal bearing housing. For some applications, first outer tube 140 and/or second outer tube 142 is coupled to the proximal bearing housing (e.g., using an adhesive). For example, first outer tube 140 may be coupled to an inner surface of the proximal bearing housing, and second outer tube 142 may be coupled to an outer surface of the proximal bearing housing. Typically, purging fluid is passed between first outer tube 140 and second outer tube 142, e.g., as described with reference to FIG. 15B of WO 24/057252 to Tuval, which is incorporated herein by reference.

Referring now to distal bearing housing 118H, for some applications, distal coupling portion 31 of frame 34 is coupled to an outer surface of distal bearing housing 118H, e.g., via a snap-fit mechanism. For example, the outer surface of a proximal-most portion 119 of the distal bearing housing may include a snap-fit mechanism to which distal coupling portion 31 of frame 34 is coupled. For some applications, distal bearing 118 is disposed within the proximal-most portion 119 of the distal bearing housing, as shown in FIG. 5A. As described hereinabove, for some applications, pump-outlet tube 24 extends to the distal end of frame 34 and defines lateral blood-inlet openings 108. For some such applications, a coupling portion 41 (e.g., a tubular coupling portion) extends distally from the pump-outlet tube, and the coupling portion is coupled to the distal bearing housing in order to anchor the distal end of the pump-outlet tube. For some applications, an intermediate portion 123 of the distal bearing housing defines a ridged or a threaded outer surface, to which coupling portion 41 of the pump-outlet tube is coupled (e.g., via an adhesive). For some applications, the outer surface is ridged in order to enhance bonding between the distal bearing housing and coupling portion 41 of the pump-outlet tube. For some applications, the outer surface is threaded in order to enhance bonding between the distal bearing housing and coupling portion 41 of the pump-outlet tube and to facilitate the application of adhesive between the outer surface and coupling portion 41 of the pump-outlet tube. For some applications, a distal portion 121 of the distal bearing housing is configured to stiffen a region of distal-tip element 107 into which the distal end of shaft 92 moves (e.g., axial-shaft-receiving tube 126, or a portion thereof). Typically, distal-tip element 107 is coupled to an outer surface of distal portion 121 of the distal bearing housing (e.g., via adhesive). For some applications, at least a portion of the outer surface of distal portion 121 of the distal bearing housing is ridged and/or threaded in order to enhance bonding between distal-tip element 107 and the distal bearing housing.

As described above, axial shaft 92 is radially stabilized via proximal radial bearing 116 and distal radial bearing 118. In turn, the axial shaft, by passing through lumen 62 defined by the impeller, radially stabilizes the impeller with respect to the inner surface of frame 34 and inner lining 39, such that even a relatively small gap between the outer edge of the blade of the impeller and inner lining 39 (e.g., a gap that is as described above) is maintained, during rotation of the impeller, as described hereinabove. Typically, the impeller itself is not directly disposed within any radial bearings or thrust bearings. Rather, bearings 116 and 118 act as radial bearings with respect to the axial shaft.

In some embodiments, pump-head portion 27 (and more generally ventricular assist device 20) does not include any thrust bearing that is configured to be disposed within the subject's body and that is configured to oppose thrust generated by the rotation of the impeller. For some applications, one or more thrust bearings are disposed outside the subject's body (e.g., within motor unit 23, shown in FIG. 1A), and opposition to thrust generated by the rotation of the impeller is provided solely by the one or more thrust bearings disposed outside the subject's body. For some applications, a mechanical element and/or a magnetic element is configured to maintain the impeller within a given range of axial positions. For example, a magnet that is disposed at the proximal end of the drive cable (e.g., outside the subject's body) may be configured to impart axial motion to the impeller, and/or to maintain the impeller within a given range of axial positions.

In alternate embodiments, axial shaft 92 is omitted, and the impeller is instead coupled to a distal portion of drive cable 130; for example, the drive cable may pass through lumen 62 of the impeller. In other words, the distal portion of the drive cable may function as an axial shaft. It should thus be understood that throughout the present description, the distal portion of the drive cable, which may also be referred to as an “axial shaft,” may substitute for axial shaft 92.

Typically, the space between delivery catheter 143 and delivery tube 142 functions as an aortic-pressure sensing channel. During operation of the left ventricular assist device, the distal end of the delivery catheter is typically disposed in the subject's descending aorta, such that this channel is exposed to the aortic bloodstream and aortic blood pressure.

Reference is now made to FIGS. 5D, 5E, and 5F, which are schematic illustrations of coupling element 65, in accordance with some embodiments. As described hereinabove, for some applications, proximal bushing 64 of impeller 50 is coupled to axial shaft 92 such that the axial position of the proximal bushing with respect to the shaft is fixed, and distal bushing 58 of the impeller is slidable with respect to the shaft. For some applications, the proximal bushing is coupled to the axial shaft via coupling element 65, for example via a snap-fit mechanism. Typically, the coupling element includes a first region (or “portion”) 66 disposed around axial shaft 92, and a second region (or “portion”) 71, which may also be disposed around the axial shaft.

The coupling element is coupled to proximal bushing 64 at second region 71. This coupling may be effected via a snap-fit mechanism, as noted above. For example, second region 71 may be shaped to define one or more protrusions 19, proximal bushing 64 may be shaped to define one or more indentations 18, and the proximal bushing may couple to second region 71 by virtue of protrusions 19 snapping into indentations 18. Alternatively, the proximal bushing may be shaped to define protrusions 19, second region 71 may be shaped to define indentations 18, and the proximal bushing may couple to second region 71 by virtue of the protrusions snapping into the indentations.

The coupling element is coupled to axial shaft 92 at first region 66. For example, for some applications, the first region of the coupling element is welded to the shaft. For other applications, the coupling element (or at least first region 66) is made of a shape-memory material (e.g., a shape-memory alloy, such as nitinol or cobalt chromium). For example, the coupling element may comprise a tube of the shape-memory material that is cut to define the first and second regions. For some such applications, at least the first region of the coupling element (or the entire coupling element) is shape set to have an inner diameter that is smaller (e.g., between 0.01 and 0.1 mm smaller) than the outer diameter of the axial shaft. For example, the axial shaft may have an outer diameter of 0.9 mm and the inner diameter of the first region of the coupling element may be between 0.85 and 0.89 mm (e.g., 0.87 mm). Thus, following the placement of the first region around the axial shaft, the first region becomes radially contracted around, and thus locked in place with respect to, the axial shaft. For some applications, coupling the coupling element to the axial shaft via this method, rather than via welding, is desirable, since the coupling element and/or the axial shaft can be weakened by being heated during the welding.

For some applications, the first region of the coupling element is shaped to define one or more slits 75, e.g., by virtue of comprising a tube that defines slits 75. Slits 75 facilitate a radial expansion of the first region such that the first region is placeable around the axial shaft. Following the placement around the axial shaft, the first region may radially contract around the axial shaft, as described above.

Slits 75 may incorporate various features for facilitating the expansion of first region 66. For example, in some embodiments, one or more of slits 75 are open-ended slits 75o, each of which has an open end. Open-ended slits 75o may include one or more proximally-open slits 75op, which are open at the proximal end of first region 66, and/or one or more distally-open slits 75od, which are open at the distal end of the first region. Optionally, the length L0 of each of the open-ended slits may be 5-40 percent of the length L1 of the coupling element. Alternatively or additionally to open-ended slits 75°, one or more of slits 75 may be closed-ended slits 75c, each of which does not have any open end. In some embodiments, as shown in FIGS. 5D-5F, closed-ended slits 75c alternate with open-ended slits 75o around the circumference of first region 66.

During manufacture of the blood pump, first region 66 is placed around axial shaft 92 such that, as described above, the first region becomes radially contracted around the axial shaft. Typically, in addition to first region 66, second region 71 is placed around the axial shaft.

Subsequently to coupling the coupling element to the axial shaft, the impeller is coupled to the axial shaft, by coupling proximal bushing 64 to the second region of the coupling element. As described above, this coupling may be performed via a snap-fit mechanism; for example, protrusions 19 may be snapped into indentations 18. Thus, as the axial shaft rotates, the blades of the impeller rotate, thereby pumping blood of the subject.

In alternate embodiments, second region 71 is coupled to distal bushing 58 (e.g., via a snap-fit mechanism, as described), such that the distal bushing is fixed in place with respect to the axial shaft, and proximal bushing 64 is slidable along the axial shaft.

Reference is now made to FIGS. 6A and 6B, which are schematic illustrations of ventricular assist device 20, the device including inner lining 39 that lines the inside of frame 34 that houses impeller 50, in accordance with some embodiments.

For some applications, inner lining 39 lines the inside of frame 34 (e.g., by virtue of being bonded to the frame), in order to provide a smooth inner surface (e.g., a smooth inner surface having a substantially circular cross-sectional shape) through which blood is pumped by impeller. Typically, by providing a smooth surface, the covering material reduces hemolysis that is caused by the pumping of blood by the impeller, relative to if the blood were pumped between the impeller and struts of frame 34. For some applications, inner lining 39 includes a polyurethane, polyester, and/or silicone. Alternatively or additionally, the inner lining includes polyethylene terephthalate (PET) and/or polyether block amide (e.g., PEBAX®).

Typically, the inner lining is disposed over the inner surface of at least a portion of central cylindrical portion 38 of frame 34. For some applications, pump-outlet tube 24 also covers central cylindrical portion 38 of frame 34 around the outside of the frame, for example, such that pump-outlet tube 24 and inner lining 39 overlap over at least 50 percent of the length of the inner lining, for example, over the entire length of the cylindrical portion of frame 34, e.g., as shown in FIG. 6A. For some applications, there is only partial overlap between pump-outlet tube 24 and inner lining 39, e.g., as shown in FIG. 6B. For example, pump-outlet tube 24 may overlap with inner lining 39 along less than 50 percent (e.g., along less than 25 percent) of the length of the inner lining. For some such applications, during insertion of ventricular assist device 20 into the subject's body, the impeller is advanced distally within frame 34, such that the impeller is not disposed within the area of overlap between the pump-outlet tube and the inner lining, such that there is no longitudinal location at which the impeller, pump-outlet tube 24, frame 34, and inner lining 39 all overlap with each other. As shown in FIGS. 6A and 6B, for some applications, a single axially-facing blood-inlet opening 108 is defined at the distal end of the pump-outlet tube and/or the inner lining. Alternatively, the inner lining is disposed over the inner surface of at least a portion of central cylindrical portion 38 of frame 34, and the pump-outlet tube extends to the distal end of the frame and defines a plurality of lateral blood-inlet openings 108. Such applications are described in further detail hereinbelow with reference to FIGS. 7A-7D, for example.

Typically, over the area of overlap between inner lining 39 and pump-outlet tube 24, the inner lining is shaped to form a smooth surface (e.g., in order to reduce hemolysis, as described hereinabove), and pump-outlet tube 24 is shaped to conform with the struts of frame 34 (e.g., as shown in the cross-section in FIG. 6A). Further typically, the inner lining has a substantially circular cross-section (for example, due to the relatively small cell width within the central cylindrical portion of the frame, as described hereinabove with reference to FIG. 2). For some applications, over the area of overlap between inner lining 39 and pump-outlet tube 24, the pump-outlet tube and the inner lining are coupled to each other, e.g., via vacuum, via an adhesive, and/or using a heat-welding procedure, which typically includes thermoforming of the pump-outlet tube, as described below.

For some applications, inner lining 39 and pump-outlet tube 24 are made of different materials (e.g., different classes of polymer) from each other. For example, the inner lining may be made of a polyurethane, and the pump-outlet tube may be made of polyether block amide (e.g., PEBAX®). Alternatively, for example, the inner lining and the pump-outlet tube may be made of different types of the same class of polymer, e.g., different types of polyurethane. Alternatively, inner lining 39 and pump-outlet tube 24 are made of the same material as each other. For example, both the inner lining and the pump-outlet tube may be made of the same type of polyurethane (i.e., the same polyurethane polymer), or of polyether block amide (e.g., PEBAX®).

For some embodiments, regardless of whether the inner lining and pump-outlet tube are made of the same material (e.g., the same polyurethane polymer) or of different materials (e.g., different polyurethane polymers or different classes of polymer), the inner lining has a flexural modulus of more than 0.1 GPa and/or less than 2 GPa, e.g., between 0.1 GPa and 2 GPa, and the pump-outlet tube has a flexural modulus of more than 0.1 GPa and/or less than 0.8 GPa (e.g., less than 0.5 GPa), e.g., between 0.1 GPa and 0.8 GPa, or between 0.1 GPa and 0.5 GPa.

Typically, the pump-outlet tube has a flexural modulus of more than 0.1 GPa and/or less than 0.8 GPa (e.g., less than 0.5 GPa), e.g., between 0.1 GPa and 0.8 GPa, or between 0.1 GPa and 0.5 GPa. Thus, the pump-outlet tube is typically configured such as to be sufficiently flexible that that edges defined by the pump-outlet tube (e.g., the edges of blood-outlet openings 109) do not injure tissue of the subject (e.g., the aortic wall).

As described above, typically the inner lining has a flexural modulus of more than 0.1 GPa and/or less than 2 GPa, e.g., between 0.1 GPa and 2 GPa. For some applications, the inner lining has a flexural modulus of more than 0.8 GPa (e.g., more than 1 GPa) and/or less than 2 GPa (e.g., less than 1.5 GPa), e.g., between 0.8 GPa and 2 GPa, or between 1 GPa and 1.5 GPa. For some applications, by having a flexural modulus of more than 0.8 GPa (e.g., more than 1 GPa), the inner lining is configured to support frame 34 in an open configuration, when the ventricular assist device is in a deployed state. For some applications, by having a flexural modulus of more than 0.8 GPa (e.g., more than 1 GPa), the inner lining is able to perform its function of lining the frame (and, optionally, supporting the frame in an open configuration), while having a relatively small thickness, e.g., a thickness less than 0.3 mm, such that the pump-head portion of the device can be more easily contained in its radially-constrained configuration. Alternatively, the inner lining has a flexural modulus of more than 0.1 GPa and/or less than 0.8 GPa (e.g., less than 0.5 GPa), e.g., between 0.1 GPa and 0.8 GPa, or between 0.1 GPa and 0.5 GPa.

For some applications, the inner lining and the pump-outlet tube are made from the same material as one another (e.g., a type of polyurethane) and both the inner lining and the pump-outlet tube have a flexural modulus of more than 0.1 GPa and/or less than 0.8 GPa (e.g., less than 0.5 GPa), e.g., between 0.1 GPa and 0.8 GPa, or between 0.1 GPa and 0.5 GPa. Alternatively, the inner lining and the pump-outlet tube are made from different types of the same class of polymer (e.g., different types of polyurethane). For some such applications, (a) the inner lining has a flexural modulus of more than 0.8 GPa (e.g., more than 1 GPa) and/or less than 2 GPa (e.g., less than 1.5 GPa), e.g., between 0.8 GPa and 2 GPa, or between 1 GPa and 1.5 GP, and (b) the pump-outlet tube has a flexural modulus of more than 0.1 GPa and/or less than 0.8 GPa (e.g., less than 0.5 GPa), e.g., between 0.1 GPa and 0.8 GPa, or between 0.1 GPa and 0.5 GPa.

Typically, the respective melting temperatures of the inner lining and the pump-outlet tube are within 20 °C of one another, e.g., within 10 °C, or within 5 °C of one another. In particular, in embodiments in which the inner lining and the pump-outlet tube are made of the same class of polymer (e.g., different types of the same class of polymer or the same type of polymer), the respective melting temperatures of the inner lining and the pump-outlet tube are within 20 °C of one another, e.g., within 10 °C, or within 5 °C of one another. The similar melting temperatures facilitate heat welding the inner lining to the pump-outlet tube, as described below.

Further typically, the respective glass-transition temperatures of the inner lining and the pump-outlet tube are within 20 °C of one another, e.g., within 10 °C, or within 5 °C of one another. In particular, in embodiments in which the inner lining and the pump-outlet tube are made of the same class of polymer (e.g., different types of the same class of polymer or the same type of polymer), the respective glass-transition temperatures of the inner lining and the pump-outlet tube are within 20 °C of one another, e.g., within 10 °C, or within 5 °C of one another. The similar melting temperatures facilitate heat welding the inner lining to the pump-outlet tube, as described below.

Typically, the pump-outlet tube is fixed over cylindrical portion 38 of frame 34 by virtue of being bonded or otherwise coupled to the cylindrical portion of the frame and/or to inner lining 39.

For some applications, the inner lining is directly bonded to the inner surface of the frame before the pump-outlet tube is bonded to the outside of the frame. It is noted that, by bonding the inner lining directly to the inner surface of the frame (rather than simply bonding the inner lining to the pump-outlet tube and thereby sandwiching the frame between the inner lining to the pump-outlet tube), any air bubbles, folds, and other discontinuities in the smoothness of the surface provided by the inner lining are typically avoided.

For example, in some embodiments, the inner lining, which is shaped as a tube, is placed over a mandrel having the desired diameter of the inner lining. The mandrel is then heated to a temperature that is higher than the glass-transition temperature of the inner lining but below the melting temperature of the inner lining. The heat causes the inner lining to shrink around the mandrel, such that the desired diameter of the inner lining is obtained. Subsequently, the frame (typically, the central cylindrical portion of the frame) is placed over the inner lining, and pressure is applied to the frame while the assembly of the mandrel, inner lining, and frame is heated in an oven. The heat and pressure cause the frame to bond to the inner lining.

For some applications, similar techniques to those described hereinabove for enhancing bonding between the elastomeric film and the helical elongate elements of the impeller, are used to enhance bonding between the inner lining and the inner surface of the frame. For example, in some applications, initially, the frame is treated so as to enhance bonding between the inner lining and the inner surface of the frame. For some applications, the treatment of the frame includes applying a plasma treatment to the frame (e.g., to the inner surface of the frame), dipping the frame in a coupling agent that has at least two functional groups that are configured to bond respectively with the frame and with the material from which the inner lining is made (e.g., a silane solution), dipping the frame in a solution that contains the material from which the inner lining is made (e.g., a polyurethane solution), and/or spraying such a solution over the inner surface of the frame. For some applications, the inner lining is made of an elastomeric material (e.g., a polyurethane) and the coupling agent is a silane solution, such as a solution of n-(2-aminoethyl)-3-aminopropyltrimethoxysilane, with the silane containing a first functional group (e.g., (OH)) which is configured to bond with the frame (which is typically made of an alloy, such a nitinol), and the silane containing a second functional group (e.g., (NH2)) which is configured to bond with the elastomeric material.

Subsequently to the inner lining having been bonded to the frame, a portion of pump-outlet tube 24 is placed around the outside of the frame, and heat and pressure are applied. Typically, at this stage, the assembly is heated to a heat-welding temperature that is above the glass-transition temperatures of at least one of the pump-outlet tube 24 and inner lining 39, such that pump-outlet tube 24 is heat welded to the inner lining. Typically, a heat-welding temperature is selected such that it is high enough to cause at least one of the inner lining and the pump-outlet tube to soften so as to become welded to the other portion, yet is not so high so as to melt either one of these components.

As noted above, in some embodiments in which the inner lining and the pump-outlet tube are made of the same class of polymer (e.g., different types of the same class of polymer or the same type of polymer), the respective melting temperatures of the inner lining and the pump-outlet tube are within 20 °C of one another, e.g., within 10 °C, or within 5 °C of one another. In some embodiments in which the inner lining and the pump-outlet tube are made of the same class of polymer (e.g., different types of the same class of polymer or the same type of polymer), the respective glass-transition temperatures of the inner lining and the pump-outlet tube are within 20 °C of one another, e.g., within 10 °C, or within 5 °C of one another. Advantageously, the similar melting temperatures and/or glass-transition temperatures facilitate selecting a heat-welding temperature that is high enough to cause both the inner lining and the pump-outlet tube to soften so as to become welded to one another, yet is not so high so as to melt either one of these components.

In some embodiments, during the heat-welding process, the frame is heated from inside the frame, using the mandrel. Typically, while the frame is heated, an outer tube (which is typically made from silicone) applies pressure to pump-outlet tube 24 that causes pump-outlet tube 24 to be pushed radially inwardly, in order to cause the pump-outlet tube to conform with the shapes of the struts of the frame, as shown in the cross-section of FIG. 6A. For some applications, the mandrel is shorter than the inner lining, such that margins are left outside of the mandrel at each of the ends of the inner lining. Thus, the inner lining acts as a shield to protect the pump-outlet tube from being overheated and damaged. In other words, the margins prevent the mandrel from coming into direct contact with the frame and/or the pump-outlet tube. In other embodiments, the heat-welding process is performed in an oven.

For some applications, following the heat welding, the combination of the frame, the inner lining, and the portion of pump-outlet tube 24 disposed around the frame is shape set to a desired shape and desired dimensions using shape setting techniques that are known in the art.

Reference is now made to FIG. 6C, which is a schematic illustration of an inner lining that includes an extension 39e extending proximally beyond cylindrical portion 38 of frame 34, in accordance with some embodiments. For some applications, the inner lining extension 39e is not coupled to the inner surface of frame 34, but rather, the material that comprises the extension is free to flap within the blood flow that is generated by the impeller. In some cases, the inner lining extension increases the efficiency of pumping of the blood by the impeller, for example by rectifying non-linear flow paths of the blood that are generated by the pumping of the impeller.

Reference is now made to FIGS. 7A-7D, which are schematic illustrations of pump-outlet tube 24 or a portion thereof, the pump-outlet tube being configured to define lateral blood-inlet openings 108 at a distal end thereof, in accordance with some embodiments. For some applications, the pump-outlet tube extends substantially until the distal end of distal conical portion 40 of frame 34. For such applications, the pump-outlet tube typically defines a distal conical portion 46 which is distally facing, i.e., facing such that the narrow end of the cone is distal with respect to the wide end of the cone. Typically, the pump-outlet tube includes coupling portion 41 (e.g., a tubular coupling portion, as shown), which extends distally from the pump-outlet tube. As described hereinabove, the coupling portion is coupled to the distal bearing housing in order to anchor the distal end of the pump-outlet tube.

For some applications (not shown), the pump-outlet tube defines two to four lateral blood-inlet openings. Typically, for such applications, each of the blood-inlet openings defines an area of more than 20 square mm (e.g., more than 30 square mm), and/or less than 60 square mm (e.g., less than 50 square mm), e.g., 20-60 square mm, or 30-50 square mm. Alternatively or additionally, the outlet tube defines a greater number of smaller blood-inlet openings 108, e.g., more than 10 blood-inlet openings, more than 10 blood-inlet openings, more than 100 blood-inlet openings, more than 200 blood-inlet openings, or more than 300 blood-inlet openings, e.g., 50-100 blood-inlet openings, 100-300 blood-inlet openings, or 300-500 blood-inlet openings. For some applications, the blood-inlet openings are sized such as (a) to allow blood to flow from the subject's left ventricle into the tube and (b) to block structures from the subject's left ventricle from entering into the frame. Typically, for such applications, the distal conical portion 46 of pump-outlet tube 24 is configured to reduce a risk of structures from the left ventricle (such as chordae tendineae, trabeculae carneae, and/or papillary muscles) entering into frame 34 and potentially being damaged by the impeller and/or the axial shaft, and/or causing damage to the left ventricular assist device. Therefore, for some applications, the blood-inlet openings are shaped such that, in at least one direction, the widths (or spans) of the openings are less than 1 mm, e.g., 0.1-1 mm, or 0.2-0.6 mm. By defining such a small width (or span), it is typically the case that structures from the left ventricle (such as chordae tendineae, trabeculae carneae, and/or papillary muscles) are blocked from entering into frame 34. For some such applications, each of the blood-inlet openings defines an area of more than 0.05 square mm (e.g., more than 0.1 square mm), and/or less than 3 square mm (e.g., less than 1 square mm), e.g., 0.05-3 square mm, or 0.1-1 square mm. Alternatively, each of the blood-inlet openings defines an area of more than 0.1 square mm (e.g., more than 0.3 square mm), and/or less than 5 square mm (e.g., less than 1 square mm), e.g., 0.1-5 square mm, or 0.3-1 square mm.

Typically, the portion of the pump-outlet tube that defines the blood-inlet openings has a porosity of more than 40 percent, e.g., more than 50 percent, more than 60 percent, or more than 70 percent (where porosity is defined as the percentage of the area of this portion that is porous to blood flow). Thus, on the one hand, the blood-inlet openings are relatively small (in order to prevent structures of the left ventricular from entering the frame), but on the other hand, the porosity of the portion of the pump-outlet tube that defines the blood-inlet openings is relatively high, such as to allow sufficient blood flow into the pump-outlet tube.

For some applications, each of the blood-inlet openings has a circular or a polygonal shape. For some applications, each of the blood-inlet openings has a hexagonal shape, as shown in FIGS. 7A-7D. Typically, using openings having a hexagonal shape allows the portion of the pump-outlet tube that defines the blood-inlet openings to have a relatively high porosity (e.g., as described hereinabove), while providing the portion of the pump-outlet tube that defines the blood-inlet openings with sufficient material between the blood-inlet openings to prevent tearing and/or stretching of the material. As shown in FIG. 7B, for some applications, a width W of gaps between adjacent hexagonal (or other polygonal) holes is more than 0.01 mm (e.g., more than 0.02 mm), and/or less than 0.2 mm (e.g., less than 0.15 mm), for example, 0.01-0.2 mm, or 0.02-0.15 mm. For some applications, the distance D between opposing sides of each of the hexagons (or other types of polygons) is more than 0.1 mm (e.g., more than 0.2 mm) and/or less than 0.8 mm (e.g., less than 0.6 mm), e.g., 0.1-0.8 mm, or 0.2-0.6 mm. As indicated in FIG. 7B, typically each of the polygons encloses a circle (such that any structure that cannot pass through such a circle would be unable to pass through the polygon). Typically, the diameter of the circle enclosed by the polygon is the equivalent of distance D, e.g., more than 0.1 mm (e.g., more than 0.2 mm) and/or less than 0.8 mm (e.g., less than 0.6 mm), e.g., 0.1-0.8 mm, or 0.2-0.6 mm.

The scope of the present disclosure includes having uniformly sized and/or shaped lateral blood-inlet openings (e.g., circular, rectangular, polygonal, and/or hexagonal lateral blood-inlet openings). Similarly, the scope of the present disclosure includes a distal conical portion 46 of the pump-outlet tube that defines lateral blood-inlet openings being arranged such that the distal conical portion has a uniform porosity, with the porosity being substantially uniform over different regions of the distal conical portion.

The scope of the present disclosure further includes having non-uniformly sized and/or shaped lateral blood-inlet openings (e.g., circular, rectangular, polygonal, and/or hexagonal lateral blood-inlet openings), disposed in any arrangement along the distal conical portion 46 of the pump-outlet tube. Similarly, the scope of the present disclosure includes a distal conical portion 46 of the pump-outlet tube that defines lateral blood-inlet openings being arranged such that the distal conical portion has a non-uniform porosity, with the porosity varying over different regions of the distal conical portion. For some applications, the shapes and/or sizes of the lateral blood-inlet openings, and/or the porosity of the distal conical portion, is varied such as to account for varying blood flow dynamics at different regions of the distal conical portion. Alternatively or additionally, the shapes and/or sizes of the lateral blood-inlet openings, and/or the porosity of the distal conical portion, is varied such as to account for changes in the shape of the distal conical portion along its length.

For some applications, along distal conical portion 46 of pump-outlet tube 24, the thickness of the polymeric material from which the pump-outlet tube is made is greater than the thickness in other regions of the pump-outlet tube (e.g., within the central cylindrical portion and/or the proximal conical portion of the pump-outlet tube). For some such applications, pump-outlet tube 24 is manufactured in this manner in order to prevent tearing of the tube within the distal conical portion 46, which defines blood-inlet openings 108, and may (in some cases) be at greater risk of tearing than other portions of the pump-outlet tube.

Reference is now made to FIG. 7D, which is an enlarged schematic illustration of the interface between the distal end of pump-outlet tube 24 and distal-tip element 107. Typically, the pump-outlet tube includes a coupling portion 41 (e.g., a tubular coupling portion, as shown), which extends distally from the pump-outlet tube. As described hereinabove, the coupling portion is coupled to distal bearing housing 118H in order to anchor the distal end of the pump-outlet tube. Also as described hereinabove, typically, the pump-outlet tube is coupled to the outside of the central cylindrical portion of the frame. For some applications, distal conical portion 46 of the pump-outlet tube is not itself bonded to distal conical portion 40 of the frame. Rather, distal conical portion 46 of the pump-outlet tube is held in place with respect to distal conical portion 40 of the frame, by virtue of coupling portion 41 being coupled to distal bearing housing 118H and the pump-outlet tube being coupled to the outside of the central cylindrical portion of the frame. Alternatively, the distal conical portion 46 of the pump-outlet tube is directly coupled to distal conical portion 40 of the frame (e.g., via heat shrinking).

As described hereinabove, for some applications, coupling portion 41 is coupled to the outer surface of portion 123 of distal bearing housing 118H. For some applications, coupling portion 41 defines a hole 111 (e.g., toward the distal end of the coupling portion), as shown in FIG. 7D. For some applications, an adhesive is applied between coupling portion 41 and the outer surface of portion 123 of distal bearing housing 118H, via the hole. For some applications, the outer surface of portion 123 of distal bearing housing 118H is threaded. Typically, the threaded outer surface allows the adhesive to gradually and uniformly spread between coupling portion 41 and the outer surface of portion 123 of distal bearing housing 118H. Further typically, the coupling portion is transparent, such that the spread of the adhesive is visible through the coupling portion. Therefore, for some applications, once the adhesive has sufficiently spread between coupling portion 41 and the outer surface of portion 123 of distal bearing housing 118H (e.g., once the outer surface of portion 123 has been covered with the adhesive), application of the adhesive is terminated.

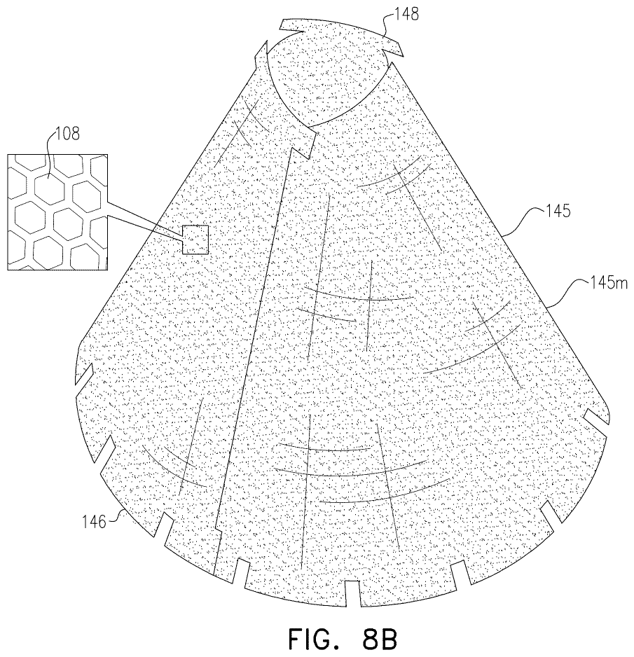

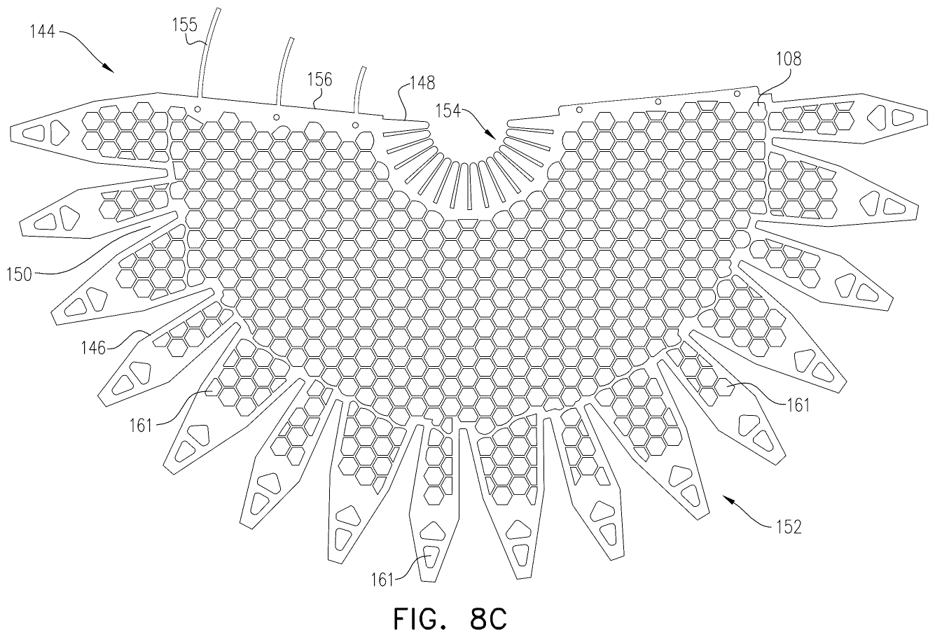

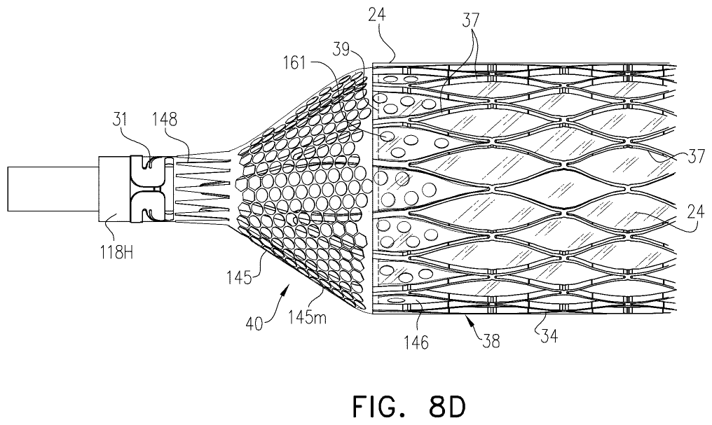

Reference is now made to FIG. 8A, which is a schematic illustration of a sheet 144 of material, such as a polyether block amide, a polyether ether ketone, or another polymer, and to FIG. 8B, which is a schematic illustration of a frustoconical inlet guard 145 formed from sheet 144, in accordance with some embodiments.

In some embodiments, sheet 144 is provided for forming inlet guard 145, which provides at least some of the function of distal portion 46 (FIG. 7D) of the pump-outlet tube. Blood-inlet openings 108, which may have any of the properties described above with reference to distal portion 46 of the pump-outlet tube, are formed in sheet 144, e.g., via laser cutting. Subsequently, sheet 144 is rolled so as to form the frustoconical inlet guard. Thus, this method of forming the inlet guard takes advantage of the fact that it is easier to form holes in a flat piece of material, rather than a three-dimensional structure.

As shown in FIG. 8A, the shape of sheet 144 is generally that of a section of a torus, comprising a longer arced proximal end 152, a shorter arced distal end 154, and opposing lateral edges 156. To roll the sheet, one lateral edge 156 is coupled (e.g., bonded) to the surface of the sheet near the opposite lateral edge, as indicated by a rolling indicator 158. One or more tabs extending from the sheet may facilitate this rolling, as further described below with reference to FIG. 8C. Following the formation of the inlet guard, the inlet guard is coupled to the pump-outlet tube, e.g., to the distal end of cylindrical portion 44 (FIG. 1C).

In some embodiments, multiple proximal flaps 146 are formed in the sheet of material, e.g., by cutting slits 150 in the proximal end of the sheet so as to define flaps 146. The inlet guard thus comprises a frustoconical main body 145m, which is shaped to define the blood-inlet openings, and proximal flaps 146. The inlet guard is coupled to the pump-outlet tube by coupling proximal flaps 146 to the pump-outlet tube. Advantageously, flaps 146 help prevent folding or creasing in the inlet guard.

In some embodiments, the inlet guard is also coupled to inner lining 39 (FIG. 7A). For example, as shown in FIG. 8D, which is described below, proximal flaps 146 may be coupled to the inner lining, such that the proximal flaps are sandwiched between the inner lining and the pump-outlet tube.

As a specific example, for some applications, the inner lining and the pump-outlet tube are heat welded to one another, e.g., as described above with reference to FIGS. 6A-6B, while the proximal flaps are between the inner lining and the pump-outlet tube. In some such embodiments, to protect the inlet guard from degradation during the heat-welding process, the heat-welding temperature (to which the inner lining and the pump-outlet tube are heated) is lower than the glass-transition temperature of the inlet guard but higher than the glass-transition temperature of at least one of the inner lining and the pump-outlet tube (and in some embodiments, higher than the glass-transition temperature of both of the inner lining and the pump-outlet tube). Furthermore, as described above, the heat-welding temperature is typically lower than the respective melting points of the inner lining and the pump-outlet tube, such that the pump-outlet tube is bonded to the inner lining without deformation of the inner lining or pump-outlet tube.

In some embodiments, the inlet guard is made of the same material as the inner lining and/or the pump-outlet tube, such as a polyurethane (e.g., Pellethane®) or a polyether ether ketone. In other embodiments, the inlet guard is made of a different material. In some such embodiments, the glass-transition temperature of the material of which the inlet guard is made is higher than the respective glass-transition temperatures of each of the inner lining and the pump-outlet tube. The heat-welding temperature is lower than the glass-transition temperature of the material but higher than the respective glass-transition temperatures of each of the inner lining and the pump-outlet tube.

For example, in some applications, the inner lining is made of a polyurethane (e.g., Pellethane®), the pump-outlet tube is made of polyether block amide (e.g., PEBAX®), and the inlet guard is made of a polyether ether ketone. The heat-welding temperature is lower than the glass-transition temperature of the polyether ether ketone but higher than the respective glass-transition temperatures of each of the polyurethane and polyether block amide. Alternatively, the inner lining and pump-outlet tube are made of the same type or different types of polyurethane, and the inlet guard is made of a polyether ether ketone or polyether block amide (e.g., PEBAX®). The heat-welding temperature is lower than the glass-transition temperature of the polyether ether ketone or polyether block amide (e.g., PEBAX®) but higher than the glass-transition temperature(s) of the polyurethane(s).

In addition to being coupled to the pump-outlet tube, the inlet guard is fixed over the distal portion of the frame. For example, in some embodiments, the inlet guard is coupled, e.g., bonded, to the distal portion of the frame, e.g., using a combination of heat and pressure as described above, with reference to FIG. 6A, for the coupling of the pump-outlet tube to the frame. Alternatively or additionally, as shown in FIG. 8D, the inlet guard is fixed over distal portion 40 of the frame by fixing proximal flaps 146 at least partly over central portion 38 of the frame. For example, the proximal flaps may be sandwiched between inner lining 39 and pump-outlet tube 24 over the central portion of the frame. Alternatively or additionally, the inlet guard is fixed over the distal portion of the frame by coupling the inlet guard to distal bearing housing 118H and/or to coupling portion 31 of the frame. For example, in some embodiments, multiple distal flaps 148 are formed in sheet 144, and the inlet guard is coupled to the bearing housing and/or to coupling portion 31 by coupling distal flaps 148 to the bearing housing and/or to coupling portion 31.

Reference is now made to FIG. 8C, which is a schematic illustration of sheet 144, in accordance with some embodiments. Reference is also made to FIG. 8D, which is a schematic illustration of frustoconical inlet guard 145, which is formed from sheet 144 as described above with reference to FIGS. 8A-8B, fixed over distal portion 40 of frame 34, in accordance with some embodiments.

In some embodiments, proximal flaps 146 are shaped such that, when the proximal flaps are fixed at least partly over central portion 38 of the frame, the proximal flaps do not overlap any of struts 37 of the frame. Thus, advantageously, the proximal flaps do not overly interfere with the coupling of the pump-outlet tube to the frame, and do not overly enlarge the diameter of the pump head. For example, the proximal flaps may be shaped such that, when the proximal flaps are fixed at least partly over the central portion of the frame, the proximal flaps fit between struts 37 while abutting the struts. Thus, advantageously, despite not overlapping the struts, the proximal flaps provide a large surface area for the coupling of the inlet guard to the pump-outlet tube.

Alternatively or additionally, proximal flaps 146 are shaped to define multiple flap openings 161, and the pump-outlet tube and the inner lining are heat welded to one another at least partly via flap openings 161. In other words, as the pump-outlet tube is heated during the heat-welding procedure, the pump-outlet tube passes through flap openings 161 and bonds to the inner lining. Typically, the pump-outlet tube also bonds to the inner lining between the proximal flaps.

In some embodiments, at least some flap openings 161, such as those flap openings at a distal portion of the proximal flaps, are sized and shaped similarly to blood-inlet openings 108. One advantage of such embodiments is ease of manufacture. Another advantage is that even if the pump-outlet tube does not completely cover the proximal flaps, the uncovered portion of the flaps does not compromise the functionality of the inlet guard.

In some embodiments, one or more tabs 155 extend from sheet 144, e.g., from a lateral edge 156 of the sheet (as shown in FIG. 8C). Sheet 144 is rolled by pulling tabs 155. Subsequently to rolling the sheet, the tabs are removed (e.g., cut) from the sheet.

It is noted that embodiments described above with reference to FIGS. 8A-8D are applicable even to cases in which the inlet guard is manufactured from a tube, rather than from a sheet of material. Examples of such embodiments include the sandwiching of the proximal flaps in the heat welding process, the flap openings, the higher glass-transition temperature of the inlet guard, the shaping of the proximal flaps to avoid overlapping the frame struts, and the coupling of the inlet guard (e.g., via the distal flaps) to the distal bearing housing.

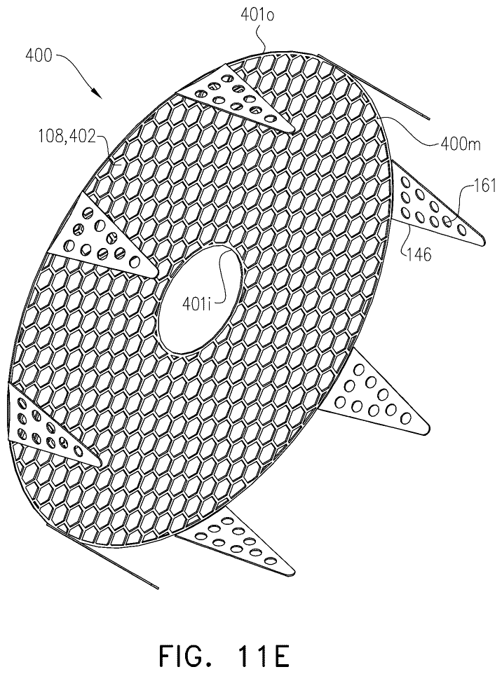

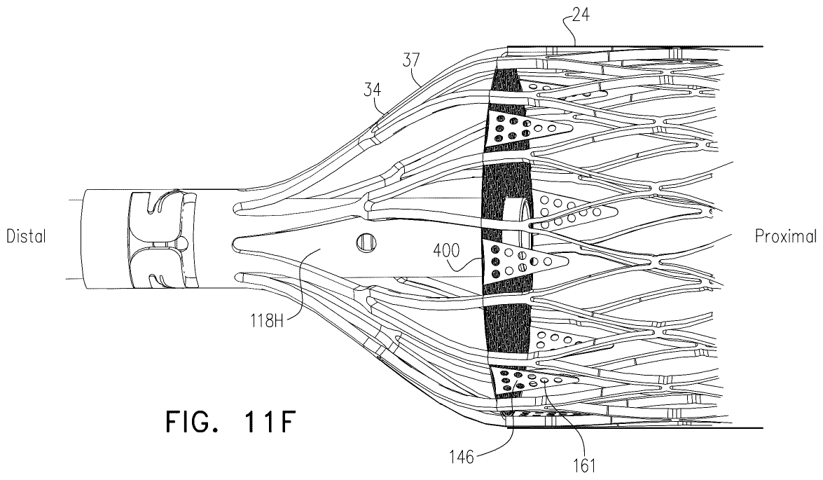

Furthermore, these embodiments are applicable even to cases in which the main body of the inlet guard is flat and is disposed within the frame, as further described below with reference to FIGS. 11E-11F.

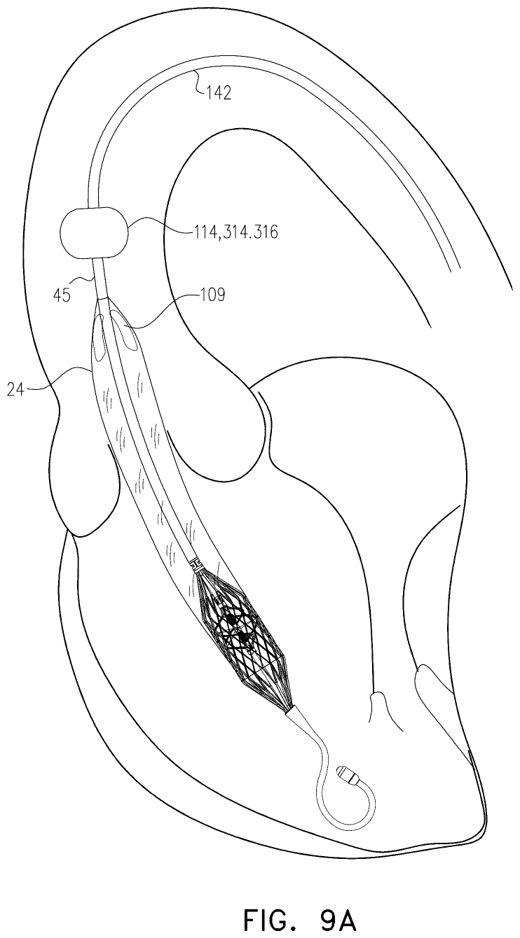

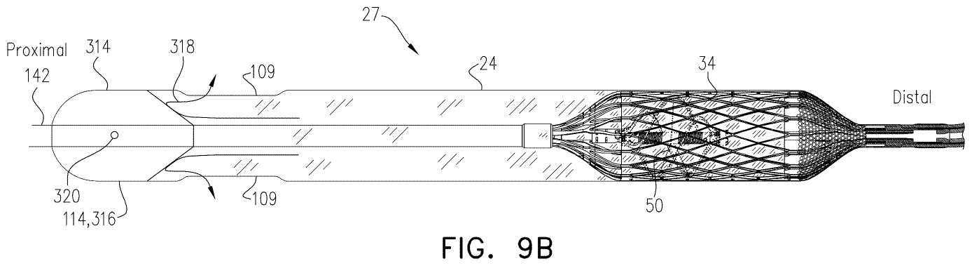

Reference is now made to FIG. 9A, which is a schematic illustration of an expandable element 314 surrounding delivery tube 142, in accordance with some embodiments. Reference is also made to FIG. 9B, which is a schematic illustration of pump-head portion 27, in accordance with some embodiments.

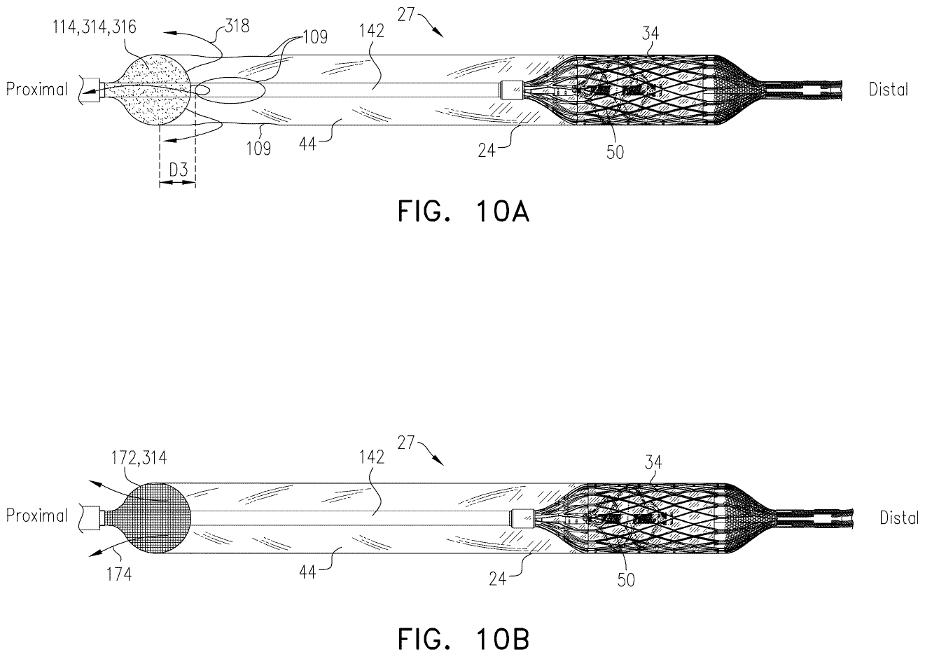

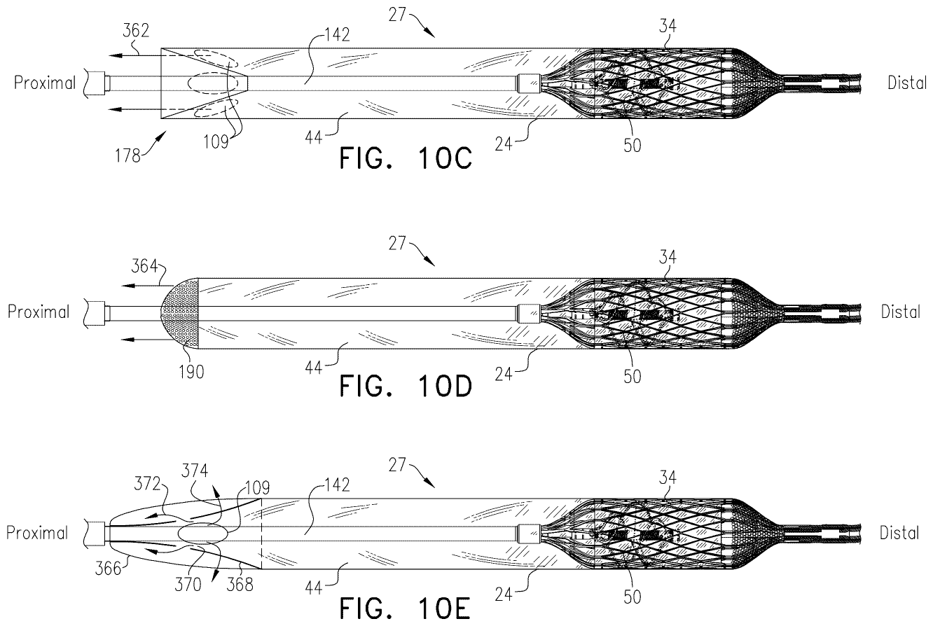

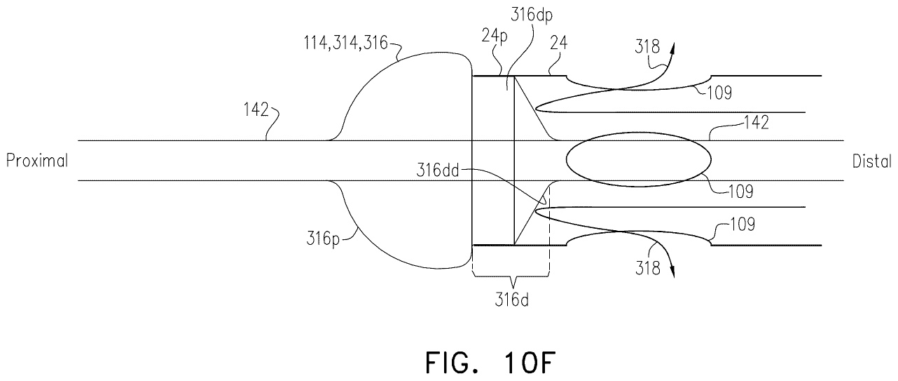

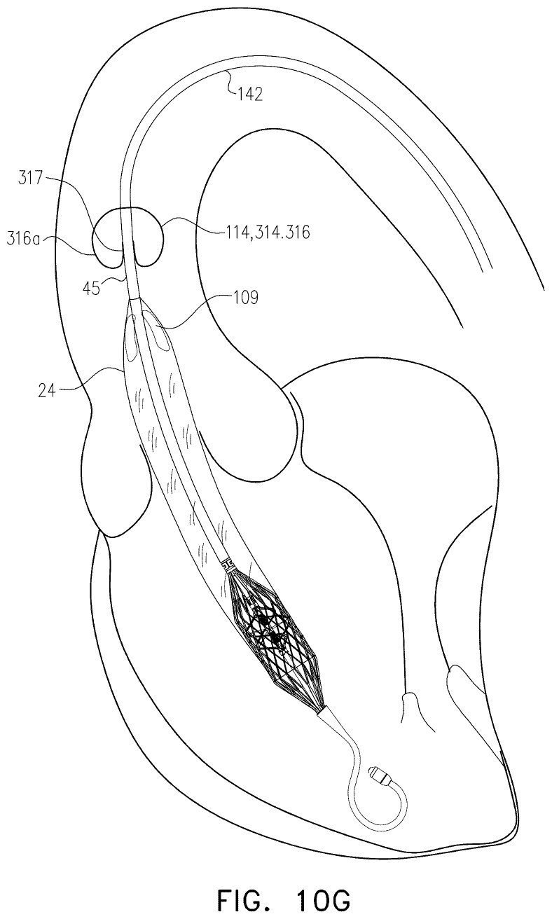

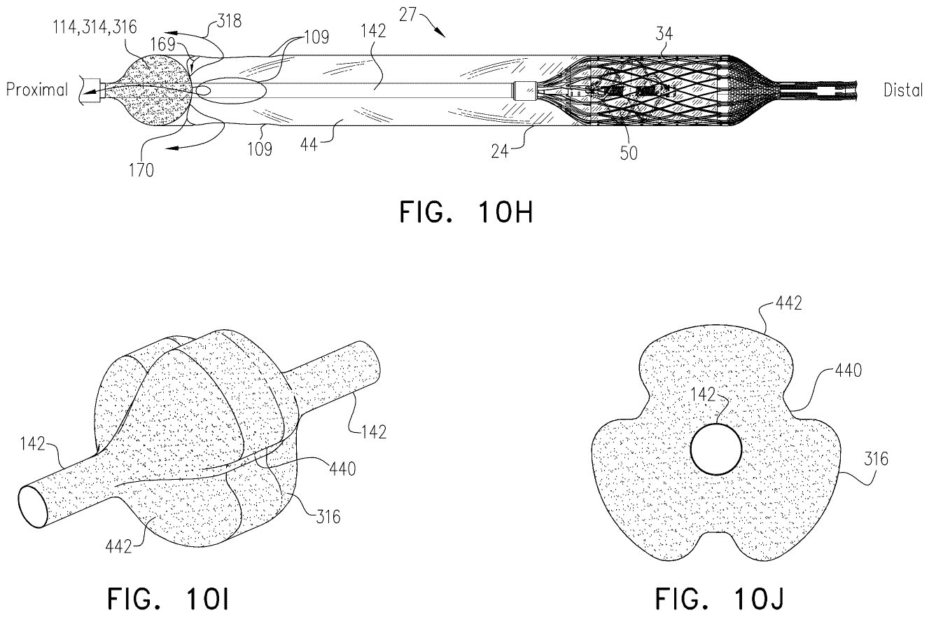

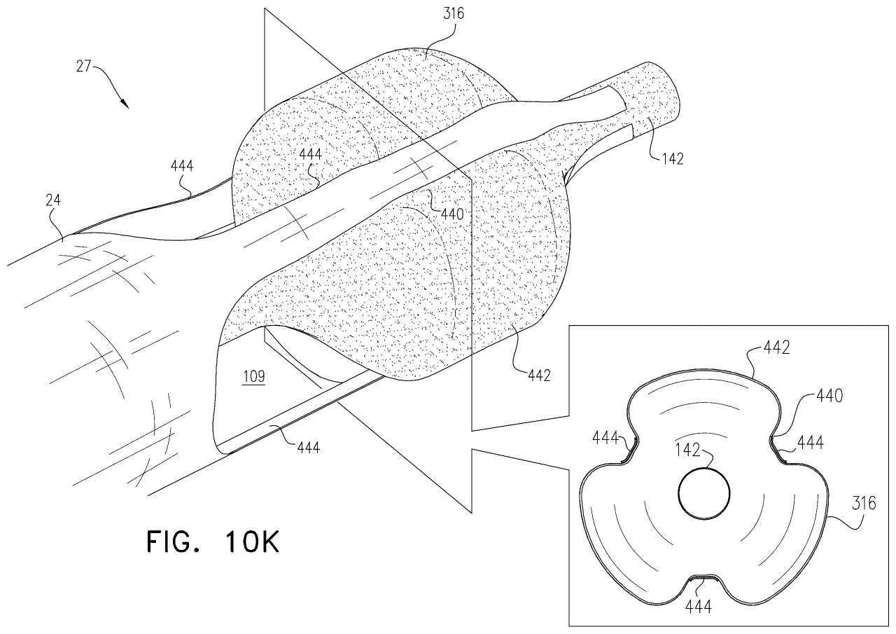

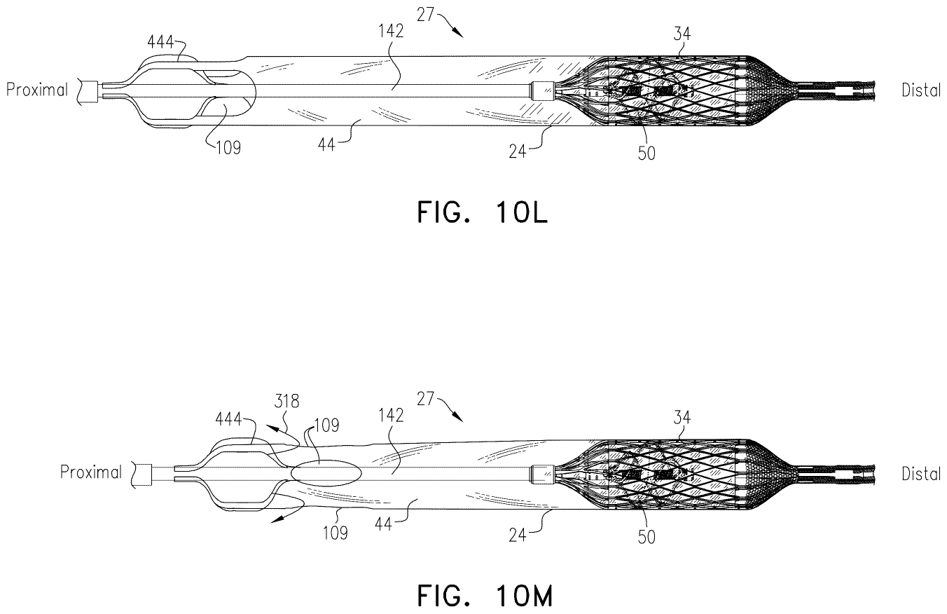

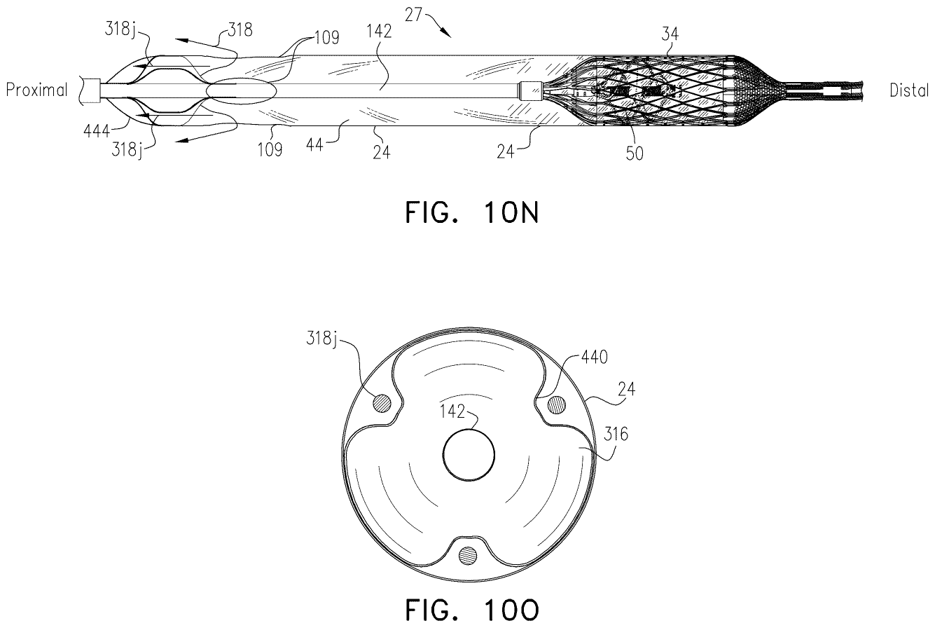

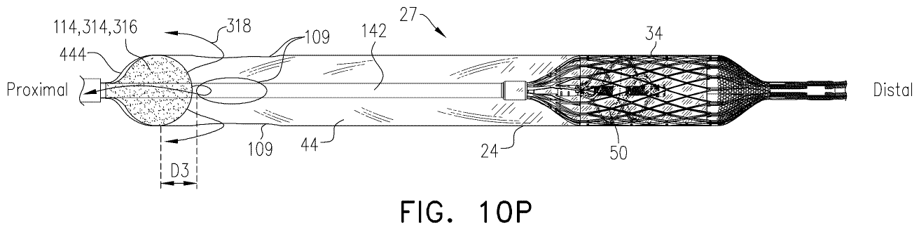

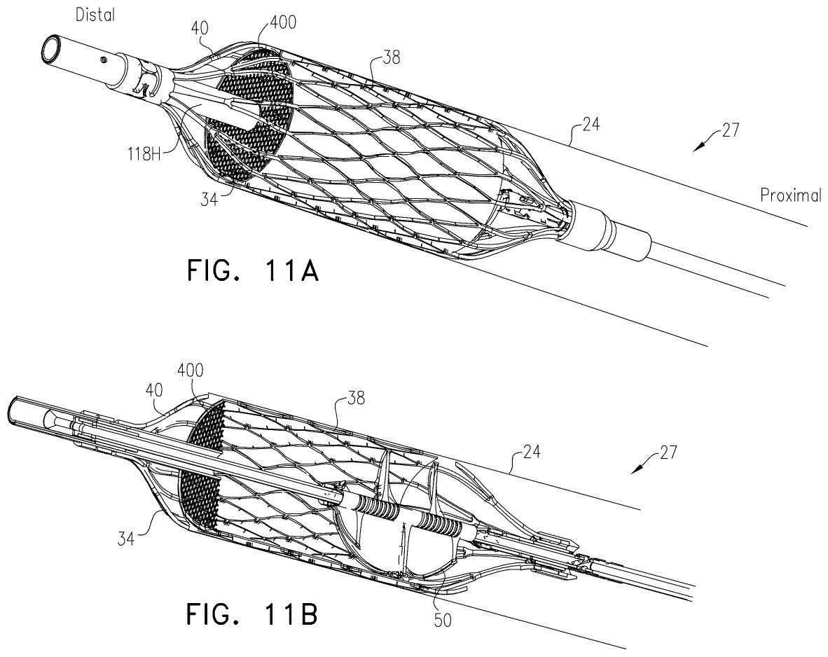

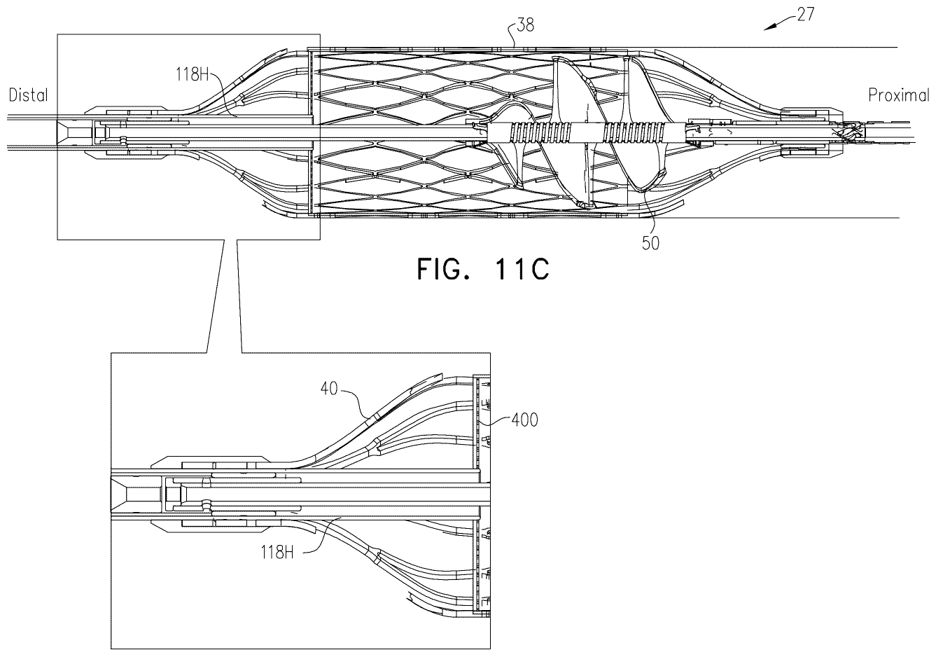

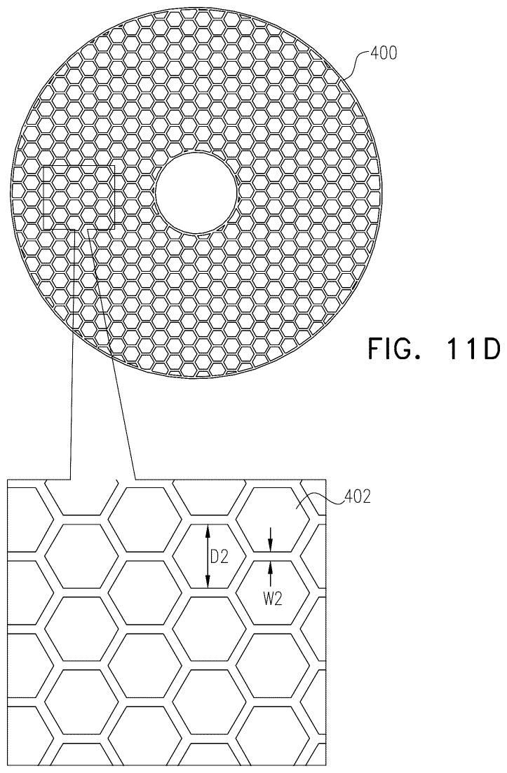

In some embodiments, as shown in FIGS. 9A-9B (and in FIGS. 10A-10B, FIGS. 10F-10H, and FIG. 10K-10P, which are described below), expandable element 314 surrounds delivery tube 142. In some such embodiments, expandable element 314 comprises an expandable stent or expandable braided element. Alternatively, expandable element 314 comprises an inflatable element 316 (e.g., a balloon 114). For some applications, inflatable element 316 is inflated using a fluid (e.g., air or saline) that is pumped through the ventricular-assist device. For example, in some embodiments, as shown in FIG. 9B, the wall of the delivery tube is shaped to define one or more openings 320, and inflatable element 316 surrounds openings 320 such that a fluid flowing, via the openings, from the delivery tube into the inflatable element inflates the inflatable element. Typically, the inflating fluid includes purging fluid, which, distally to openings 320, purges the interface between the axial shaft and any stationary bearings (including radial and/or thrust bearings) that don't rotate with the axial shaft. Alternatively or additionally, the inflating fluid comes from a separate, dedicated supply.

Typically, expandable element 314 is proximal to blood-outlet openings 109, with the length of the delivery tube between expandable element 314 and the blood-outlet openings being less than 30 mm.

In some embodiments, as shown in FIG. 9A, expandable element 314 is entirely proximal to the pump-outlet tube. For example, in some embodiments, expandable element 314 is disposed slightly proximally to the interface between delivery tube 142 and pump-outlet tube 24 (as shown in FIG. 9A). For example, in some embodiments, expandable element 314 is slightly proximal to tubular coupling portion 45 or to strips 29 (FIG. 1D).

In other embodiments, as shown in FIG. 9B, expandable element 314, at least when expanded, is disposed at least partly within, e.g., entirely within, pump-outlet tube 24. In some such embodiments, the pump-outlet tube does not comprise a conical proximal portion, and is not coupled directly to delivery tube 142.

Expandable element 314 is configured to protect the aortic wall from injury, e.g., by inhibiting the edges of blood-outlet openings 109, which are sometimes sharp, from contacting the wall of the aorta. Alternatively or additionally, expandable element 314 is configured to center a portion of the ventricular assist device (e.g., the portion of delivery tube 142 near the pump-outlet tube) within the aorta. Expandable element 314 is configured to perform these functions by abutting the aortic wall.

Alternatively or additionally, expandable element 314 is shaped to direct the blood through blood-outlet openings 109, as indicated in FIG. 9B by blood-flow arrows 318. For example, in some embodiments, the distal end of the expandable element has a width that decreases moving distally, e.g., the distal end is frustoconical, such that the blood is directed by the distal end of the expandable element, at an angle, through the blood-outlet openings. Alternatively or additionally, the expandable element has an angled and/or a curved surface that is configured to direct the blood flow in this manner. For some applications, by directing blood flow in this manner, the overall pumping efficiency of the device is increased, relative to if the device would not include an expandable element.

It is noted that expandable element 314 may be combined with any of the embodiments of pump-outlet tube 24 described with reference to FIGS. 33A-33C of WO 24/057252 to Tuval, which is incorporated herein by reference.

In some applications, a ventricular assist device that comprises expandable element 314 is selected for longer-term treatments, such as treatment for cardiogenic shock, due to the protection provided by the expandable element. On the other hand, for shorter-term treatments, such as a percutaneous coronary intervention, a device without expandable element 314, e.g., as shown in FIG. 1D, is selected, given that over shorter periods, there is less chance of injury to the aortic wall. Alternatively or additionally, other factors are considered when deciding whether to select a device that comprises expandable element 314.

Reference is now made to FIGS. 10A, 10B, 10C, and 10D, which are schematic illustrations of pump-head portion 27, in accordance with some embodiments.

FIG. 10A is similar to FIG. 9B, in that FIG. 10A shows expandable element 314, which comprises an inflatable element 316 (e.g., a balloon 114), surrounding delivery tube 142 and disposed at least partly (e.g., entirely) within pump-outlet tube 24, proximally to the blood-outlet openings. Furthermore, in FIG. 10A, as in FIG. 9B, the expandable element acts as a blood flow director, by directing blood from the proximal end of the pump-outlet tube through the blood outlet openings, as indicated by blood-flow arrows 318. FIG. 10A differs from FIG. 9B, however, in the shape of the expandable element; in particular, in FIG. 10A, the expandable element is more spherically-shaped.

In some embodiments, regardless of the shape of the expandable element, the expandable element is coupled to the lateral wall of the pump-outlet tube, which is shaped to define blood-outlet openings 109, within 0.5-5 mm (e.g., within 1-3 mm) of the blood-outlet openings. In other words, in some embodiments, the axial distance D3 between the distal-most portion of the lateral wall that is coupled to the expandable element and the proximal-most portion of the edge of each of the blood-outlet openings is between 0.5 and 5 mm (e.g., within 1 and 3 mm). Advantageously, this range is small enough such that any blood directed away from the expandable element can quickly exit the blood-outlet openings, yet is large enough such that the expandable element does not push the edges of the blood-outlet openings, which are sometimes sharp, into the wall of the aorta.

Referring to FIG. 10B, for some applications, expandable element 314 is a porous expandable element, such as an expandable cage or stent 172, that surrounds delivery tube 142 within, and/or immediately proximally to, the pump-outlet tube, such that the blood is pumped through the porous expandable element. For example, in some embodiments, the porous expandable element is disposed at the proximal end of the pump-outlet tube, and the proximal end of the pump outlet tube is coupled to delivery tube 142 via the porous expandable element. For some such applications, the pump-outlet tube does not define blood-outlet openings 109 (FIG. 10A). Rather, blood flows out of the pump-outlet tube exclusively via the porous expandable element, as indicated by blood-flow arrows 174. For some applications, the porous expandable element comprises a structure made of a shape-memory alloy, such as a laser-cut shape-memory alloy and/or a braided shape-memory alloy.

Referring to FIG. 10C, for some applications, a proximal portion 178 of pump-outlet tube 24, which defines blood-outlet openings 109, is folded inwardly toward the distal end of the pump-outlet tube. Typically, as shown, proximal portion 178 is folded inwardly such that the blood-outlet openings direct blood proximally (substantially parallel to the axis of outer tube 142) rather than radially outwardly (away from the axis of outer tube 142), as indicated by blood-flow arrows 362. For some applications, proximal portion 178 is folded inwardly such that the blood-outlet tube forms a protective layer between blood flowing out of the blood-outlet openings and the walls of the subject's aorta.

Referring to FIG. 10D, for some applications, the blood-outlet openings 109 are defined by a substantially proximally-facing surface 190 of the pump-outlet tube, rather than being defined by a lateral surface of the pump-outlet tube. Typically, for such applications, blood flow from the pump-outlet tube is axially directed, as indicated by blood-flow arrows 364.

Reference is now made to FIG. 10E, which is a schematic illustration of pump-outlet tube 24 that defines a blood-flow chamber 366 at its proximal end, in accordance with some embodiments. For some applications, the blood-flow chamber is defined by an internal membrane 368 that is disposed within the proximal end of the pump-outlet tube and that defines holes 370 therethrough. Blood flows into the blood-flow chamber via holes 370, as indicated by blood-flow arrows 372. Subsequently, the blood flows out of the blood-flow chamber and into the subject's aorta via blood-outlet openings 109 (which are generally as described hereinabove), as indicated by blood-flow arrows 374. Typically, by virtue of the blood flowing through the blood-flow chamber, the blood-flow chamber inflates such as to center a portion of the left-ventricular assist device (e.g., the delivery tube, and in particular, the portion of the delivery tube near pump-outlet tube 24) within the aorta, by contacting the aorta wall. Typically, the internal membrane is shaped so as to direct the blood flow out of the blood-outlet openings.

For some applications, internal membrane 368 is a continuation of pump-outlet tube 24, and the internal membrane is covered with an external membrane, which defines the blood-outlet openings, and which forms the external surface of blood-flow chamber 366. For such applications, the proximal end of the blood-outlet tube is shaped so as to direct the blood flow out of the blood-outlet openings.

Typically, the combination of the proximal end of the blood-outlet tube and an additional membrane (whether an internal membrane or an external membrane) is configured to define blood-flow chamber 366, which typically functions as described above. In general, the scope of the present disclosure includes any structure that provides a blood-flow chamber disposed at a proximal end of the pump-outlet tube, the blood-flow chamber defining (a) holes 370 via which blood is pumped into the blood-flow chamber and (b) blood-outlet openings 109 configured to be disposed within the aorta, via which the blood flows out of the blood-flow chamber and into the aorta.

Reference is now made to FIG. 10F, which is a schematic illustration of inflatable element 316, such as balloon 114, disposed at the proximal end of pump-outlet tube 24, in accordance with some embodiments.

FIG. 10F is similar to FIG. 9B with respect to the features noted above with reference to FIG. 10A, but differs with respect to the shape of inflatable element 316. In particular, in FIG. 10F, inflatable element 316 comprises two portions: a distal inflatable-element portion 316d, which is coupled to the inside of the lateral wall of pump-outlet tube 24 (thus, typically, sealing the proximal end of the pump-outlet tube), and a proximal inflatable-element portion 316p, which is wider than the pump-outlet tube and is disposed proximally to the pump-outlet tube.

In general, it is desired to avoid contact between the wall of the aorta and the portion 24p of the lateral wall of the pump-outlet tube at which the pump-outlet tube is coupled to the inflatable element. Proximal inflatable-element portion 316p is configured to protect the aortic wall from such contact. Furthermore, typically, proximal inflatable-element portion 316p is configured to center delivery tube 142 within the aorta.

Typically, distal inflatable-element portion 316d is at least partly cylindrical, to facilitate coupling the distal inflatable-element portion to the pump-outlet tube. For example, in some embodiments, a proximal portion 316dp of distal inflatable-element portion 316d is cylindrical. Alternatively or additionally, the distal inflatable-element portion is shaped to direct the blood through blood-outlet openings 109, as indicated by blood-flow arrows 318. For example, in some embodiments, a distal portion 316dd of distal inflatable-element portion 316d has a width that decreases moving distally, e.g., distal portion 316dd is frustoconical, such that distal portion 316dd directs the blood.

Reference is now made to FIG. 10G, which is a schematic illustration of inflatable element 316, such as balloon 114, disposed proximally to pump-outlet tube 24, in accordance with some embodiments.

FIG. 10G is similar to FIG. 9A. A difference, however, is that in FIG. 10G, a distal portion 316a of the inflatable element is everted inwardly and is coupled to delivery tube 142 at an interface 317. Thus, advantageously, the coupling of the pump-outlet tube to the delivery tube does not interfere with the coupling of the inflatable element to the delivery tube.

In some embodiments, as shown in FIG. 10G, the pump-outlet tube comprises tubular coupling portion 45, via which the pump-outlet tube is coupled to delivery tube 142. Distal portion 316a of the inflatable element is coupled to the delivery tube proximally to the tubular coupling portion, e.g., at a distance of less than 20 mm, such as less than 10 mm, from the tubular coupling portion.

Reference is now made to FIG. 10H, which is a schematic illustration of pump-head portion 27, in accordance with some embodiments. FIG. 10H is similar to FIG. 9B with respect to the features noted above with reference to FIG. 10A.

In some embodiments, to form blood-outlet openings 109, portions of the lateral wall of pump-outlet tube 24 are cut. In some such embodiments, rather than removing these portions (i.e., rather than cutting closed curves in the lateral wall such that the portions are completely detached from the rest of the wall), flaps 170 are cut in the lateral wall (i.e., open curves are cut in the lateral wall so as to define flaps 170). Flaps 170 are then folded inwardly, as indicated by folding indicators 169, and coupled to the inflatable element.

As noted above with reference to FIG. 9A, the edges of the blood-outlet openings are sometimes sharp, and there is a risk of these edges contacting the wall of the aorta. Inflatable element 316 can mitigate this risk by abutting the aortic wall, thereby keeping the edges at a distance from the wall. However, there is still some risk that, in certain situations, the inflatable element will push the edges into the wall. One way to mitigate this risk is to distance the inflatable element from edges, as described above with reference to FIG. 10A. Alternatively or additionally, flaps 170 mitigate this risk by eliminating the portions of the edges adjacent to the inflatable element, e.g., using the technique described with reference to FIG. 10H.

Reference is now made to FIGS. 10I-10J, which are schematic illustrations of inflatable element 316 from oblique and frontal perspectives, respectively, in accordance with some embodiments.

In some embodiments, inflatable element 316 is shaped to define one or more (e.g., three, four, or 6-10) grooves 440, the function of which is described with reference to subsequent figures. Typically, by virtue of being shaped to define grooves 440, inflatable element 316 comprises lobes 442, the number of lobes 442 being the same as the number of grooves. Grooves 440 run between lobes 442.

Reference is now made to FIGS. 10K-10M, which are schematic illustrations of pump-head portion 27, in accordance with some embodiments.

By way of introduction, it is noted that to couple pump-outlet tube 24 to inflatable element 316, an adhesive is typically required. However, the adhesive may cause the inflatable element to become less flexible and/or injure the aortic wall in some cases. Hence, in some embodiments, the pump-outlet tube is coupled (e.g., heat welded) to delivery tube 142, rather than to the inflatable element. In such embodiments, pump-outlet tube 24 comprises multiple tabs 444, which extend proximally from the proximal portion of the pump-outlet tube, and which are coupled to the delivery tube proximally to the inflatable element.

In some embodiments, as shown in FIGS. 10K-10M, tabs 444 pass through grooves 440. Advantageously, the grooves reduce the profile of the device, such that the tabs do not contact the aortic wall. Furthermore, the grooves hold the tabs in place, thereby reducing the risk of blood stagnating between the tabs and the inflatable element. Typically, the tabs are coupled to the delivery tube immediately proximally to the inflatable element, thereby further reducing the risk of stagnant blood.

In some embodiments, as shown in FIGS. 10K-10L, inflatable element 316 is offset proximally from the proximal portion of the pump-outlet tube so as to define multiple blood-outlet openings 109 between tabs 444 and between the proximal portion of the pump-outlet tube and the inflatable element. The blood exits the proximal portion of the pump-outlet tube via these blood-outlet openings. Advantageously, it is typically not necessary to cut separate blood-outlet openings in the lateral wall of the pump-outlet tube.

In other embodiments, as shown in FIG. 10M, the inflatable element contacts (e.g., is coupled to) the proximal portion of the blood-outlet tube, and the blood exits via blood-outlet openings 109 in the lateral wall of the pump-outlet tube.

Reference is now made to FIG. 10N, which is a schematic illustration of pump-head portion 27, in accordance with some embodiments. Reference is also made to FIG. 10O, which shows a schematic frontal view of the proximal end of pump-head portion 27, in accordance with some embodiments.

FIGS. 10N-10O show an alternate solution for reducing the risk of stagnant blood. In particular, inflatable element 316 is disposed within the proximal portion of the pump-outlet tube, such that tabs 444 are mostly or entirely proximal to the inflatable element. Typically, the tabs are coupled to delivery tube 142 between 0.5 and 6 mm, e.g., 1-3 mm, from the inflatable element.

As indicated by blood-flow arrows 318j, at least some of the blood exits the proximal portion of the pump-outlet tube via grooves 440. These jets of blood help reduce the risk of blood stagnating between the pump-outlet tube and the inflatable element or between the tabs and the delivery tube.

In some embodiments, as shown in FIG. 10N, the lateral wall of the pump-outlet tube is shaped to define blood-outlet openings 309, and some of the blood exits the proximal portion of the pump-outlet tube via the blood-outlet openings. In other embodiments, all the blood exits via grooves 440.

Reference is now made to FIG. 10P, which is a schematic illustration of pump-head portion 27, in accordance with some embodiments.

The embodiment shown in FIG. 10P is similar to that shown in FIG. 10A. For example, in FIG. 10P, the lateral wall of pump-outlet tube is shaped to define blood-outlet openings 109, and inflatable element 316 surrounds delivery tube 142 and is disposed at least partly (e.g., entirely) within the pump-outlet tube proximally to the blood-outlet openings. However, in FIG. 10P, multiple tabs 444 extend proximally from the proximal portion of the pump-outlet tube and are coupled to the delivery tube proximally (e.g., immediately proximally) to the inflatable element. Thus, advantageously, even if some blood flows to the proximal side of inflatable element 316 (instead of exiting the pump-outlet tube via blood-outlet openings 109), this blood can exit the pump-outlet tube via the spaces between tabs 444.

In some embodiments, as shown in FIG. 10P, the inflatable element is not shaped to define any grooves. In other embodiments, the inflatable element is shaped to define one or more grooves and the tabs pass through the grooves, e.g., as shown in FIG. 10M.

Reference is now made to FIGS. 11A, 11B, 11C, and 11D, which are schematic illustrations of portions of ventricular assist device 20, the device including an inlet guard 400 disposed inside frame 34, in accordance with some embodiments. Inlet guard 400 is shaped to define one or more holes 402, shown enlarged in FIG. 11D, which are disposed around the axial shaft and within frame 34 distally to the impeller, such that the blood flows to the impeller via holes 402. The inlet guard may be coupled to the struts of frame 34, to inner lining 39 (FIG. 4) of the frame, to the inner wall of pump-outlet tube 24, and/or to distal bearing housing 118H.

For some applications, inlet guard 400 is flat and/or is disposed such that it is perpendicular to the axial shaft (i.e., to the longitudinal axis of the frame). Thus, advantageously, the inlet guard may occupy relatively little space, and/or may provide an advantageous flow direction for the blood. Typically, the inlet guard is toric.

For some applications, the ventricular assist device includes a thrust bearing in pump-head portion 27. Typically, for such applications, the impeller does not move distally of cylindrical portion 38 of frame 34 (either during delivery of the device to the left ventricle or during operation of the device).

For some applications, the inlet guard is placed within the frame at the distal end of the central cylindrical portion of the frame or in the vicinity thereof, e.g., within 1 mm of the distal end of the cylindrical portion. This placement may simplify the assembly of the blood pump. For some applications, distal bearing housing 118H extends into the distal conical portion of the frame (e.g., until at least the end of the central cylindrical portion of the frame) and the inner edge of inlet guard 400 is couple to the distal bearing housing, as shown in FIGS. 11A-11B.

Typically, the inlet guard is polymeric, i.e., is made of a polymeric material (such as a polyurethane (e.g., Pellethane®), polyethylene terephthalate (“PET”), ultra-high-molecular-weight polyethylene (“UHMWPE”), and/or polyether block amide (e.g., Pebax®)) that is shaped to define holes 402. For some applications, the thickness of the inlet guard is more than 40 microns (e.g., more than 50 microns), and/or less than 100 microns (e.g., less than 80 microns), for example, 40-100 microns or 50-80 microns. Thus, the inlet guard may be configured to withstand pressure yet be crimpable.

Typically, for applications in which ventricular assist device 20 includes inlet guard 400 disposed inside frame 34, pump-outlet tube 24 does not extend until the distal end of distal conical portion 40 of frame 34. Moreover, pump-outlet tube 24 may have an open distal end, rather than terminating in a distal conical portion. (Thus, the inlet guard may simplify the manufacture of the blood pump.) The distal end of the pump-outlet tube may be proximal to the distal end of the distal conical portion of the frame. For example, the distal end of the pump-outlet tube may be within 1 mm of the distal end of the central cylindrical portion of frame 34, i.e., the pump-outlet tube may extend only until the end of the cylindrical portion of frame 34, or the vicinity thereof. Blood may thus flow into frame 34 via openings defined by the distal conical portion of the frame.

For some applications, holes 402 of inlet guard 400 are sized such as (a) to allow blood to flow from the subject's left ventricle into pump-outlet tube 24 and (b) to block structures from the subject's left ventricle from entering into the pump-outlet tube. Typically, for such applications, the inlet guard is configured to reduce a risk of structures from the left ventricle (such as chordae tendineae, trabeculae carneae, and/or papillary muscles) entering into pump-outlet tube 24 and potentially being damaged by the impeller and/or the axial shaft, and/or causing damage to the ventricular assist device.

For some applications, inlet guard 400 defines more than 10 holes, more than 50 holes, more than 100 holes, more than 150 holes, or more than 200 holes e.g., 50-100 holes, 100-150holes, 150-200, or 200-300 holes. For some applications, the holes are sized such as (a) to allow blood to flow from the subject's left ventricle into the tube and (b) to block structures from the subject's left ventricle from entering into the frame. Typically, for such applications, the inlet guard is configured to reduce a risk of structures from the left ventricle (such as chordae tendineae, trabeculae carneae, and/or papillary muscles) entering into the cylindrical portion of frame 34 and potentially being damaged by the impeller and/or the axial shaft, and/or causing damage to the left ventricular assist device. Therefore, for some applications, the holes are shaped such that, for each of the holes, the span of the hole in at least one direction is less than 1 mm, e.g., 0.1-1 mm, or 0.2-0.6 mm. By defining such a small width (or span), it is typically the case that structures from the left ventricle (such as chordae tendineae, trabeculae carneae, and/or papillary muscles) are blocked from entering the cylindrical portion of frame 34.

For some applications, each of the holes defines an area of more than 0.05 square mm (e.g., more than 0.1 or 0.3 square mm), and/or less than 5 square mm (e.g., less than 3 or 1 square mm), e.g., 0.05-5, 0.05-3, 0.1-1, 0.1-5, or 0.3-1 square mm.

Typically, the inlet guard has a porosity of at least 40 percent, e.g., more than 50 percent, more than 60 percent, or more than 70 percent (where porosity is defined as the percentage of the area of this portion that is porous to blood flow). Thus, on the one hand, the holes are relatively small (in order to prevent structures of the left ventricular from entering the frame), but on the other hand, the porosity of the portion of the pump-outlet tube that defines the holes is relatively high, such as to allow sufficient blood flow into the pump-outlet tube.

For some applications, each of the holes has a circular or a polygonal shape. For some applications, each of the holes has a hexagonal shape, as shown most clearly in FIG. 11D. Typically, using openings having a hexagonal shape allows the inlet guard to have a relatively high porosity (e.g., as described hereinabove), while providing the inlet guard with sufficient material between the holes to prevent tearing and/or stretching of the material.

As shown in FIG. 11D, for some applications, a width W2 of gaps between adjacent holes 402 (i.e., the distance between each pair of adjacent holes) is more than 0.01 mm (e.g., more than 0.02 mm), and/or less than 0.2 mm (e.g., less than 0.15 mm), for example, 0.01-0.2 mm, or 0.02-0.15 mm.

As further shown in FIG. 11D, for some applications, the distance D2 between opposing sides of each of the hexagons (or other types of polygons) is more than 0.1 mm (e.g., more than 0.2 mm) and/or less than 0.8 mm (e.g., less than 0.6 mm), e.g., 0.1-0.8 mm, or 0.2-0.6 mm. Typically each of the polygons encloses a circle (such that any structure that cannot pass through such a circle would be unable to pass through the polygon). Typically, the diameter of the circle enclosed by the polygon is the equivalent of distance D2, e.g., more than 0.1 mm (e.g., more than 0.2 mm) and/or less than 0.8 mm (e.g., less than 0.6 mm), e.g., 0.1-0.8 mm, or 0.2-0.6 mm.

For some applications, the frame is assembled with the inlet guard inside in the following manner. As described hereinabove, during assembly of the pump-head portion, the proximal end of frame 34 is typically open. For some applications, the inlet guard is placed through the open proximal end of the frame while being supported upon a rod (e.g., a mandrel). The inlet guard typically has an overall torus shape, with the edges of the shape defining inner and outer circles, as shown in FIG. 11D. The inner circle defined by the inlet guard is typically coupled to distal bearing housing 118H, as shown in FIGS. 11A-11B and the outer circle is coupled to struts of frame 34, to pump-outlet tube 24, and/or to inner lining 39. For some applications, the aforementioned coupling of the inlet guard to other portions of the device is performed via suturing, via hooks, via adhesive, and/or via heat fusion.

As noted above, in some embodiments, the inlet guard is coupled to the distal bearing housing, which may house a radial and/or thrust bearing. In such embodiments, typically, the distal bearing housing is partly or entirely disposed within frame 34. For example, at least 10 percent, 50 percent, or 80 percent of the length of the bearing housing may be disposed within the frame. Moreover, the distal bearing housing may extend into the frame even for applications in which the blood pump does not comprise inlet guard 400.

Reference is now made to FIG. 11E, which is a schematic illustration of inlet guard 400, in accordance with some embodiments. Reference is also made to FIG. 11F, which is a schematic illustration of inlet guard 400 disposed within frame 34, in accordance with some embodiments. (It is noted that some elements of the pump-head portion, such as axial shaft 92 and inner lining 39, are not shown in FIG. 11F.)

The embodiment of inlet guard 400 shown in FIGS. 11E-11F combines aspects of the embodiments of FIGS. 11A-11D with those of FIGS. 8A-8D. In particular, the main body 400m of the inlet guard, which is shaped to define holes 402 (which can alternatively be referred to as blood-inlet openings 108) is flat and is disposed within frame 34, e.g., such that main body 400m is perpendicular to axial shaft 92, as in FIGS. 11A-11D. For example, main body 400m may be toric, comprising an inner circular edge 401i, which is optionally coupled to the distal bearing housing, and an outer circular edge 401°, which is optionally coupled to struts 37 of frame 34, to pump-outlet tube 24, and/or to inner lining 39. In addition, as in FIGS. 8A-8D, the inlet guard comprises proximal flaps 146, via which the inlet guard is coupled to pump-outlet tube 24 and/or to the inner lining of the frame. For example, the proximal flaps may be sandwiched between the pump-outlet tube and the inner lining, e.g., via the heat welding process described above. In some embodiments (not shown), inlet guard 400 includes distal flaps 148 (e.g., as shown in FIGS. 8A-8D), which extend distally from inner edge 401i and are coupled to the distal bearing housing.

As a specific example, for some applications, the inner lining and the pump-outlet tube are heat welded to one another, e.g., as described above with reference to FIGS. 6A-6B, while the proximal flaps are between the inner lining and the pump-outlet tube. In some such embodiments, to protect inlet guard 400 from degradation during the heat-welding process, the heat-welding temperature (to which the inner lining and the pump-outlet tube are heated) is lower than the glass-transition temperature of the inlet guard but higher than the glass-transition temperature of at least one of the inner lining and the pump-outlet tube (and in some embodiments, higher than the glass-transition temperature of both of the inner lining and the pump-outlet tube). Furthermore, as described above, the heat-welding temperature is typically lower than the respective melting points of the inner lining and the pump-outlet tube, such that the pump-outlet tube is bonded to the inner lining without deformation of the inner lining or pump-outlet tube.

In some embodiments, inlet guard 400 is made of the same material as the inner lining and/or the pump-outlet tube, such as a polyurethane (e.g., Pellethane®) or a polyether ether ketone. In other embodiments, inlet guard 400 is made of a different material. In some such embodiments, the glass-transition temperature of the material of which the inlet guard is made is higher than the respective glass-transition temperatures of each of the inner lining and the pump-outlet tube. The heat-welding temperature is lower than the glass-transition temperature of the material but higher than the respective glass-transition temperatures of each of the inner lining and the pump-outlet tube.

For example, in some applications, the inner lining is made of a polyurethane (e.g., Pellethane®), the pump-outlet tube is made of polyether block amide (e.g., PEBAX®), and inlet guard 400 is made of a polyether ether ketone. The heat-welding temperature is lower than the glass-transition temperature of the polyether ether ketone but higher than the respective glass-transition temperatures of each of the polyurethane and polyether block amide. Alternatively, the inner lining and pump-outlet tube are made of the same type or different types of polyurethane, and inlet guard 400 is made of a polyether ether ketone or polyether block amide (e.g., PEBAX®). The heat-welding temperature is lower than the glass-transition temperature of the polyether ether ketone or polyether block amide (e.g., PEBAX®) but higher than the glass-transition temperature(s) of the polyurethane(s).

For some applications, inlet guard 400 is coupled to pump-outlet tube 24 and/or to the inner lining of the frame by fixing proximal flaps 146 at least partly over central portion 38 of the frame. For example, the proximal flaps may be sandwiched between inner lining 39 and pump-outlet tube 24 over the central portion of the frame.

In some embodiments, proximal flaps 146 are shaped such that, when the proximal flaps are fixed at least partly over central portion 38 of the frame, the proximal flaps do not overlap any of struts 37 of the frame. Thus, advantageously, the proximal flaps do not overly interfere with the coupling of the pump-outlet tube to the frame, and do not overly enlarge the diameter of the pump head. For example, the proximal flaps may be shaped such that, when the proximal flaps are fixed at least partly over the central portion of the frame, the proximal flaps fit between struts 37 while abutting the struts. Thus, advantageously, despite not overlapping the struts, the proximal flaps provide a large surface area for the coupling of the inlet guard to the pump-outlet tube.

Alternatively or additionally, proximal flaps 146 are shaped to define multiple flap openings 161, and the pump-outlet tube and the inner lining are heat welded to one another at least partly via flap openings 161. In other words, as the pump-outlet tube is heated during the heat-welding procedure, the pump-outlet tube passes through flap openings 161 and bonds to the inner lining. Typically, the pump-outlet tube also bonds to the inner lining between the proximal flaps.

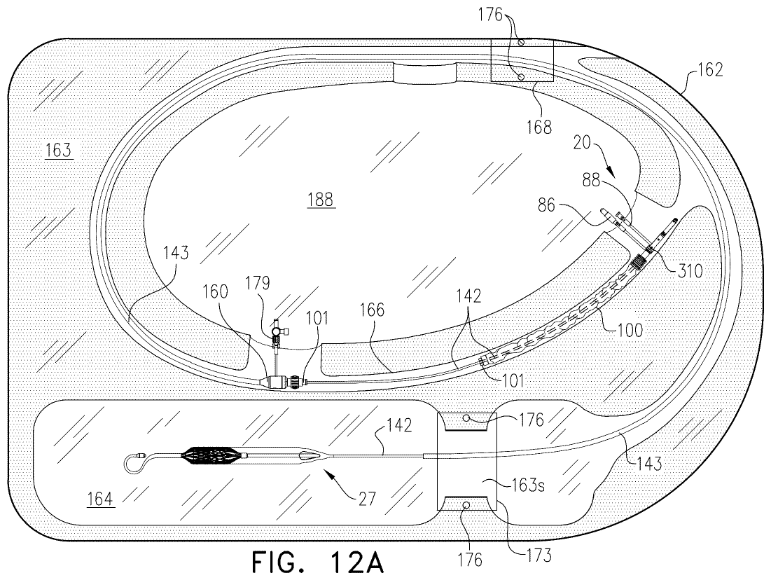

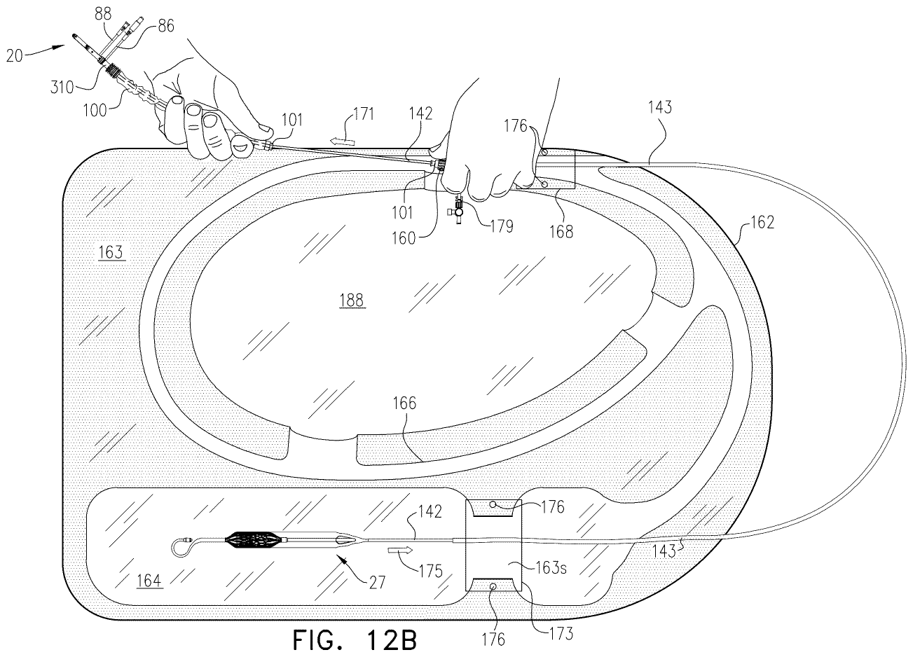

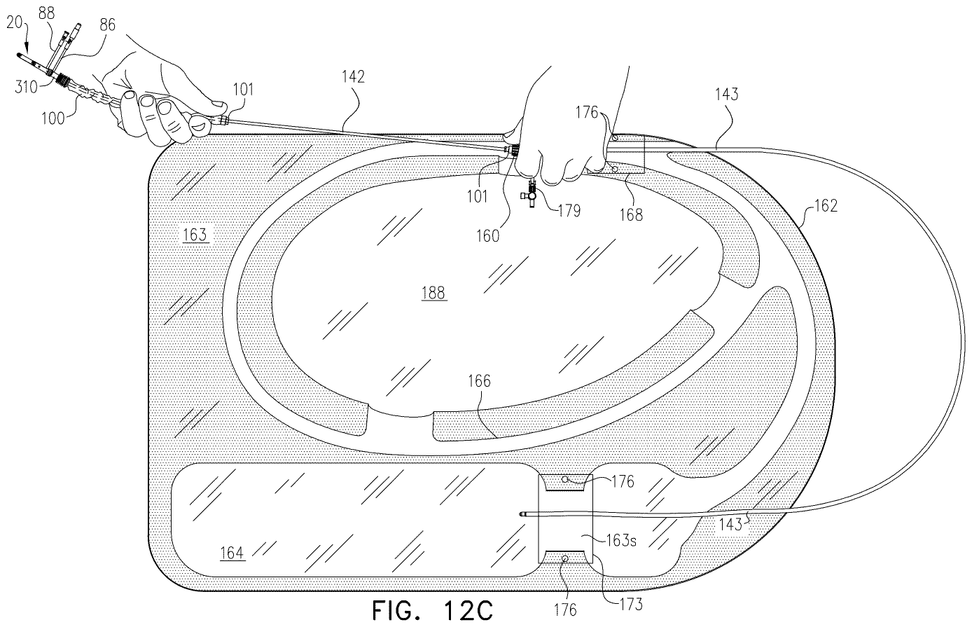

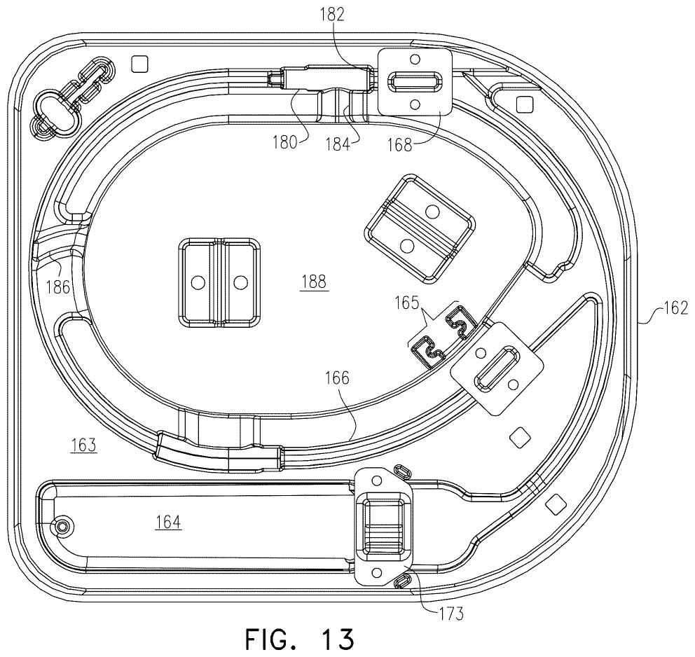

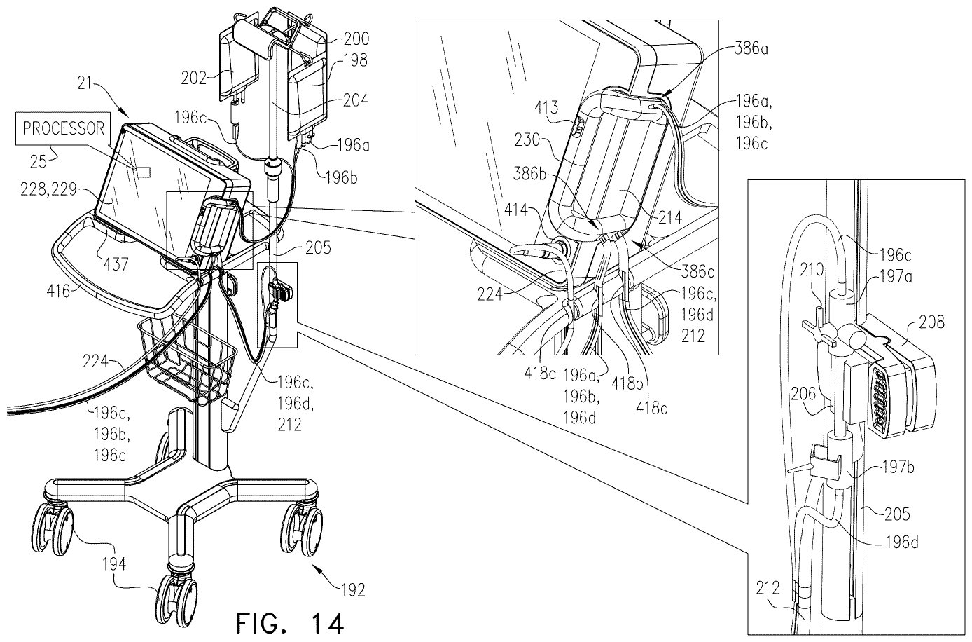

Reference is now made to FIG. 12A, which is a schematic illustration of ventricular assist device 20 packaged within packaging 162, in accordance with some embodiments.

By way of introduction, it is noted that, in some embodiments, device 20 comprises a fixation unit 160, in addition to delivery catheter 143, delivery tube 142, and pump-head portion 27. Delivery catheter 143 is coupled to fixation unit 160 distally to fixation unit 160 (such that the fixation unit is disposed at the proximal end of the delivery catheter), and delivery tube 142 passes through the fixation unit and through the delivery catheter. Fixation unit 160, which is wider than the delivery catheter (i.e., which radially protrudes from the delivery catheter) and is disposed at the proximal end of the delivery catheter, is configured to fix the position of delivery tube 142 relative to delivery catheter 143, e.g., as described with reference to FIG. 7D, FIGS. 26A-26B, or FIGS. 27A-27B of WO 24/057252 to Tuval, which is incorporated herein by reference. (The aforementioned reference uses the term “locking unit” with reference to the embodiment of fixation unit 160 shown in FIGS. 26A-26B and 27A-27B of the reference.) For example, fixation unit 160 may comprise a Tuohy Borst adapter or a clip, which, when engaged, grips the delivery tube so as to inhibit movement of the delivery tube relative to the delivery catheter. In some embodiments, fixation unit 160 comprises a pressure-sensing port 179, which is shaped to define a lumen in fluid communication with aortic pressure-sensing channel 147 (FIG. 5B). Pressure-sensing port 179 is configured to connect to a pressure sensor 206 (FIG. 14), such that the pressure sensor may sense the aortic pressure of the subject via the pressure-sensing port.

Also shown in FIG. 12A is a driven-magnet unit 310, which is described with reference to FIGS. 7A-7E of WO 24/057252 to Tuval, which is incorporated herein by reference, a sterile sleeve 100 configured to cover the proximal end of delivery tube 142, and a toric joint mechanism 101 comprising two portions configured to couple to one another: one portion at the distal end of sterile sleeve 100, and the other portion at the proximal end of fixation unit 160. Typically, driven-magnet unit 310 comprises an inlet port 86 and an outlet port 88. As further described below with reference to FIG. 15A, a purging fluid is continuously or periodically pumped into the ventricular assist device via inlet port 86 and out of the ventricular assist device via outlet port 88.

Packaging 162 comprises a tray 163, which may be made of a polymer or any other suitable material. Typically, packaging 162 further comprises a cover (not shown), configured to cover tray 163 while device 20 is packaged. The user removes the cover from the tray prior to performing the additional unpackaging and preparatory steps described below.

Tray 163 is shaped to define a chamber 164 in which pump-head portion 27 is packageable in a non-radially-constrained configuration. Further to opening the packaging, the user prepares the pump-head portion for percutaneous delivery by retracting the delivery tube, thereby retracting the pump-head portion into the delivery catheter. Advantageously, tray 163 is configured to stabilize fixation unit 160 while the pump-head portion is retracted into the delivery catheter, such that, by using the tray to stabilize the fixation unit, the user may perform the retraction in a controlled manner.

In some embodiments, the user must actively maintain the disengagement of fixation unit 160. In such embodiments, while retracting the delivery tube, the user maintains the disengagement of fixation unit 160. In other embodiments, the user simply disengages the fixation unit prior to retracting the delivery tube, or device 20 is packaged with fixation unit 160 already disengaged.

Packaging 162 further comprises a securement piece 173 coupled to the tray (e.g., reversibly coupled to the tray) adjacently to chamber 164 and configured to secure the distal end of the delivery catheter while the pump-head portion is retracted into the delivery catheter. Typically, the distal end of the delivery catheter is secured such that the distal tip of the catheter is disposed within chamber 164.

In some embodiments, tray 163 is further shaped to define a track 166 in which the delivery catheter is packageable. Securement piece 173 is typically positioned over track 166, such that the securement piece secures the distal end of the delivery catheter within the track.

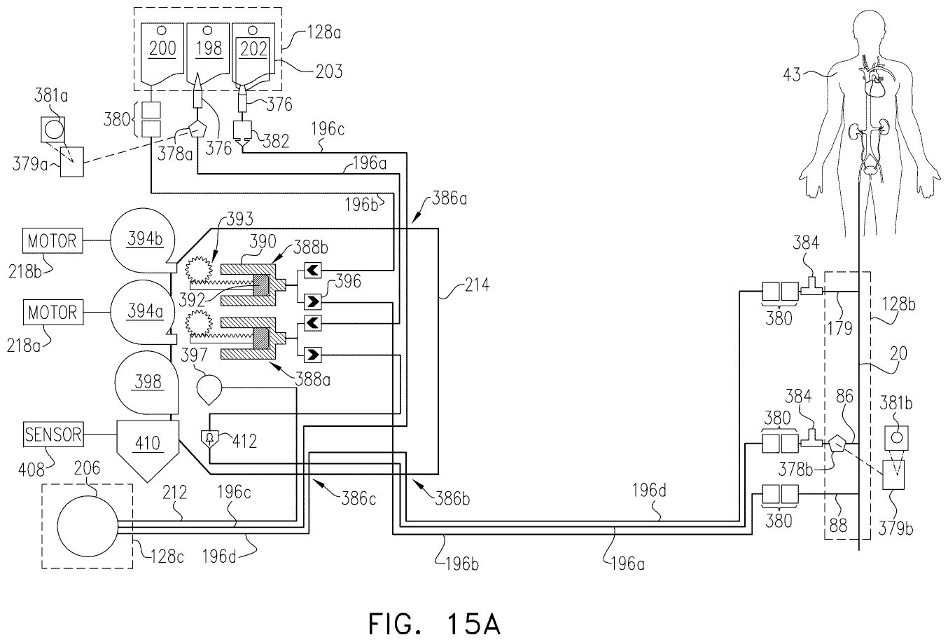

In some such embodiments, packaging 162 comprises a detachable element 168, which is reversibly coupled to tray 163 over the track and is configured to stabilize fixation unit 160 during the retraction, as further described below with reference to FIGS. 12B-12C. Alternatively or additionally, as further described below with reference to FIG. 13, track 166 comprises a widened portion 180 configured to stabilize the proximal element.

Reference is now made to FIGS. 12B-12C, which schematically illustrate a method for removing device 20 from packaging 162, in accordance with some embodiments.

In some embodiments, to remove the device, the user first slides delivery catheter 143 distally under detachable element 168 until fixation unit 160 reaches the detachable element. Next, the user pushes the fixation unit against detachable element 168, thereby stabilizing the fixation unit, while retracting delivery tube 142, as indicated in FIG. 12B by a retracting indicator 171. The retraction of the delivery tube causes pump-head portion 27 to enter the distal end of the delivery catheter, as indicated by another retracting indicator 175. Thus, the user crimps pump-head portion 27, i.e., the user places pump-head portion 27 in its radially-constrained configuration within the delivery catheter, as shown in FIG. 12C.

Following the retraction of pump-head portion 27 into the distal end of the delivery catheter, the user uncouples detachable element 168 and securement piece 173 from tray 163. For example, each of detachable element 168 and securement piece 173 may comprise one or more extensions 176 that fit within corresponding depressions in tray 163. To uncouple each of these elements from the tray, a pulling force may be applied so as to remove extensions 176 from the depressions. Following the uncoupling of detachable element 168 and securement piece 173, device 20 is removed from tray 163.

In some embodiments, following the retraction of pump-head portion 27, the user extends sleeve 100 over the proximal portion of the delivery tube and then couples the two portions of toric joint mechanism 101 to one another. Alternatively or additionally, the fixation unit is reengaged.

By securing the distal end of the catheter, securement piece 173 facilitates the retraction of pump-head portion 27 into the catheter. Furthermore, typically, chamber 164 is filled, at least partly, with a liquid, such as saline, prior to the retraction of the pump-head portion, and securement piece 173 holds the distal tip of the catheter in the liquid, thus helping prevent the distal tip of the catheter from being exposed to air during the retraction of the pump-head portion, such that no air enters the catheter.

In some embodiments, a portion 163s of the tray underneath the securement piece, which typically extends between track 166 and chamber 164, slopes downwardly in the direction of chamber 164. (The slope is described as “downward” with reference to a typical scenario in which the tray is resting upright on a horizontal surface.) Advantageously, this downward slope causes any air bubbles in chamber 164 to escape to portion 163s, such that the air bubbles don't enter the catheter. Furthermore, the liquid that is to fill chamber 164 can be poured gently down the slope into chamber 164, such that fewer bubbles are generated. Moreover, the downward slope facilitates releasing any air from the pump-outlet tube.

Reference is now made to FIG. 13, which is a schematic illustration of packaging 162, in accordance with some embodiments.

In some embodiments, track 166 comprises a widened portion 180. To stabilize fixation unit 160 (FIGS. 12A-12B) during the retraction, the user pushes the fixation unit against a wall 182 of widened portion 180. In such embodiments, detachable element 168 is omitted, or functions in a manner different from that described above. For example, in some embodiments, detachable element 168 helps secure the catheter while the device is packaged. Following the removal of the cover of tray 163, the user uncouples detachable element 168 from the tray, positions fixation unit 160 within widened portion 180 while pulling the middle portion of the catheter away from the tray, and then crimps pump-head portion 27 as described above with reference to FIGS. 12A-12C.

In some such embodiments, tray 163 is further shaped to define a side track 184 that opens into the side of widened portion 180, and the fixation unit is oriented such that pressure-sensing port 179 passes through side track 184.

It is noted that by facilitating the stabilization of the fixation unit (either via wall 182 of widened portion 180, or via detachable element 168), the tray facilitates crimping (i.e., radial compression) of the pump head (e.g., crimping of frame 34 and/or impeller 50). This is because, in order to crimp the pump head, the pump head (which is disposed at the distal end of the delivery tube 142) must be retracted into the distal end of delivery catheter 143. Typically this is done by a user pulling the proximal end of delivery tube 142 with one hand, while holding the fixation unit (which is disposed at the proximal end of the delivery catheter) with the other hand.

Typically, securement piece 173 facilitates the securement of the distal end of the delivery catheter in position during the crimping. It is typically the case that the user needs to stand in close proximity to securement piece 173 during the crimping, in order to verify that the pump head has crimped properly and that air bubbles have not entered the distal end of the catheter. However, the length of the delivery catheter is typically more than 90 cm, or more than a meter, in length. Utilizing the tray to stabilize the fixation unit allows the user to stand in close proximity to securement piece 173, while holding the fixation unit steady (by pushing it against wall 182 of widened portion 180, or against detachable element 168) and while pulling the proximal end of the delivery tube, so as to retract the pump head into the distal end of the delivery catheter.

As shown in FIGS. 12A-12C and FIG. 13, tray 163 is typically shaped to define a compartment 188, which, in some embodiments, is at least partly surrounded by track 166. Compartment 188 is configured to hold one or more tubes, cables, and/or other components that are used with device 20, such as components described below with reference to FIGS. 14 and 15A. In some embodiments, the tray is further shaped to define a groove 186, which runs outwardly from compartment 188 and through track 166. In some such embodiments, one or more longitudinal elements, such as tubes and/or cables, that are packaged in compartment 188 are looped around the catheter via groove 186, such that the user may locate these elements more easily.

Alternatively to ventricular assist device 20, packaging 162 may be used to package any other device that includes delivery catheter 143, a proximal element (such as fixation unit 160) that is disposed proximally to the delivery catheter and is wider than the delivery catheter, an elongate element (such as a tube or cable) passing through the proximal element and delivery catheter 143, and a self-expandable element coupled to the elongate element distally to the elongate element and configured for percutaneous delivery to a portion of a body of a subject while the self-expandable element is in a radially-constrained configuration within the delivery catheter.

Reference is now made to FIG. 14, which is a schematic illustration of a cart 192 for use with a ventricular assist device or any other suitable intracorporeal device, in accordance with some embodiments. Reference is also made to FIG. 15A, which is a schematic layout of a cartridge 214 and associated components, in accordance with some embodiments.

Cart 192 is configured to carry control console 21, components of purging system 17 (FIG. 1A), and/or other components described below. For example, typically, cart 192 comprises a tray 437, which is configured to carry the console. Typically, cart 192 comprises wheels 194, which facilitate transporting the cart.

Typically, one or more fluid bags hang from a cart adjunct 204, which is coupled (typically removably) to cart 192, such as to a post 205 of the cart. In some embodiments, these bags include a purging-fluid bag 198, which contains purging fluid (e.g., a glucose solution) for pumping through the device, a waste bag 200, which is configured to receive the purging fluid that exits the device, and a flushing-fluid bag 202, which contains a flushing fluid (e.g., saline) for flushing aortic pressure-sensing channel 147 (FIG. 5B). As described hereinabove, the space between delivery catheter 143 and delivery tube 142 typically functions as the aortic-pressure sensing channel. During operation of the left ventricular assist device, the distal end of the delivery catheter is typically disposed in the subject's descending aorta, such that this channel is exposed to the aortic bloodstream and blood pressure.

A pressure sensor 206, which is configured to sense the pressure in the aortic pressure-sensing channel, comprises a first fluid port 197a and a second fluid port 197b. In some embodiments, pressure sensor 206 is coupled to post 205, typically via a clip 208 that allows adjusting the height of the pressure sensor.

Console 21 comprises a chassis 420, which is configured to connect to the ventricular assist device 20, e.g., via cable 224. As described above with reference to FIG. 1A, console 21 further comprises processor 25, which is disposed within chassis 420 and is configured to control the device (e.g., to control the pumping of blood) via the connection to the device, e.g., via cable 224. Typically, the processor is further configured to control the pumping of purging fluid, the aortic pressure sensing, and/or other functionality.

As further described above with reference to FIG. 1A, typically, console 21 further comprises display 228. Processor 25 is configured to display, on display 228, information related to the pumping of blood by the blood pump, the pumping of purging fluid, the aortic pressure sensing, and/or other functionality. In some embodiments, display 228 comprises a touch screen 229, and the processor is additionally configured to receive instructions via touch screen 229. In response to the instructions, the processor controls the device, the pumping of purging fluid, the aortic pressure sensing, and/or other functionality.

Typically, console 21 is powered via an internal battery or via a connection to the mains power supply.

Typically, to facilitate preparing the device for use, multiple tubes for use with the device pass through a common cartridge (or “cassette”) 214. In some embodiments, these tubes comprise a purging-fluid tube 196a, which is configured to connect purging-fluid bag 198 to inlet port 86, a waste tube 196b, which is configured to connect waste bag 200 to outlet port 88, a flushing tube 196c, which is configured to connect flushing-fluid bag 202 to port 197a of the pressure sensor, and a pressure-sensing tube 196d, which is configured to connect port 197b of the pressure sensor to pressure-sensing port 179 (which is in fluid communication with the aortic pressure-sensing channel) such that the flushing fluid flows, via the pressure sensor, into pressure-sensing port 179. Typically, cartridge 214 is packaged in compartment 188 (FIG. 13).

In some embodiments, cartridge 214 is configured for insertion into console 21; for example, console 21 may be shaped to define a slot 230 into which cartridge 214 is insertable. Typically, in such embodiments, cartridge 214 and console 21 interact with one another following the insertion of the cartridge. Several types of such interaction are described in detail below.

As described above, the tubes that pass through cartridge 214 typically interconnect three groups of components: a first group 128a, which includes the three fluid bags, a second group 128b, which includes the fluid ports of device 20, and a third group 128c, which includes the fluid ports of pressure sensor 206 and, optionally, an electrical interface for powering the pressure sensor. First group 128a is proximal to third group 128c, which in turn is proximal to second group 128b.

Typically, to minimize the potential for human error, cartridge 214 comprises three ports that correspond to these three groups: a first port 386a, a second port 386b, and a third port 386c. In other words, the tubes pass through the cartridge such that the portions of the tubes that connect to first group 128a pass through first port 386a, the portions of the tubes that connect to second group 128b pass through second port 386b, and the portions of the tubes that connect to third group 128c pass through third port 386c. In particular, respective proximal portions of purging-fluid tube 196a, waste tube 196b, and flushing tube 196c pass through first port 386a, respective distal portions of purging-fluid tube 196a, waste tube 196b, and pressure-sensing tube 196d pass through second port 386b, and the distal portion of flushing tube 196c and the proximal portion of pressure-sensing tube 196d pass through third port 386c.

Typically, the purging system comprises a proximal air-eliminating filter 378a and a distal air-eliminating filter 378b, each of which is configured to remove air from the purging fluid. Advantageously, the two air-eliminating filters remove air more effectively, relative to a single filter, and also provide redundancy for safety purposes. More generally, it is noted that the scope of the present disclosure includes the use of two air-eliminating filters with any kind of intracorporeal device.

Typically, proximal air-eliminating filter 378a comprises an air-filtering membrane 379a shaped to define pores 381a, and distal air-eliminating filter 378b comprises another air-filtering membrane 379b shaped to define second pores 381b. Second pores 381b are smaller than first pores 381a, such that the air bubbles filtered from the purging fluid by distal air-eliminating filter 378b are smaller than those filtered by proximal air-eliminating filter 378a.

Purging-fluid tube 196a is connected to purging-fluid bag 198 via proximal air-eliminating filter 378a. For example, in some embodiments, the proximal portion of purging-fluid tube 196a is connected to proximal air-eliminating filter 378a, which is connected, via a short piece of connecting tubing, to a spike 376 configured for insertion into purging-fluid bag 198. Purging-fluid tube 196a is further connected to inlet port 86 via distal air-eliminating filter 378b. For example, in some embodiments, the distal portion of purging-fluid tube 196a is connected, via a Luer lock 380, a stopcock 384, and short pieces of connecting tubing, to distal air-eliminating filter 378b, which is connected to inlet port 86. Stopcock 384 allows direct injection of purging fluid into inlet port 86.

Typically, the proximal portion of waste tube 196b is connected to waste bag 200, and the distal portion of waste tube 196b is connected to outlet port 88, via respective Luer locks 380.

Typically, an inflation cuff 203 applies pressure (e.g., a pressure of approximately 300 mmHg) to flushing-fluid bag 202. The proximal portion of flushing tube 196c is connected to flushing-fluid bag 202 via a spike 376 and a drip chamber 382, which is used to control the flow rate of the flushing fluid. In some embodiments, the distal portion of pressure-sensing tube 196d is connected to pressure-sensing port 179 via a Luer lock 380 and a stopcock 384, which allows direct injection of flushing fluid into pressure-sensing port 179.

Typically, most of the connections described above with reference to FIG. 15A are made prior to packaging the device, such that, after the packaging is opened, relatively few additional connections are required.

In some embodiments, cartridge 214 comprises one or more pumps configured to pump the purging fluid from purging-fluid bag 198, through device 20, and into waste bag 200. For example, in some embodiments, cartridge 214 comprises a first pump 388a, which is configured to pump the purging fluid through purging-fluid tube 196a, and a second pump 388b, which is configured to pump the purging fluid through waste tube 196b. Alternatively, the cartridge comprises a single pump, which interfaces with either purging-fluid tube 196a or waste tube 196b so as to pump the purging fluid through both tubes.

In such embodiments, typically, console 21 comprises one or more motors configured to drive the pumps following the insertion of the cartridge. For example, in some embodiments, the console comprises a first motor 218a configured to drive first pump 388a via a first mechanical interface 394a, and a second motor 218b configured to drive second pump 388b via a second mechanical interface 394b.

In alternative embodiments, the console comprises the pumps. The cartridge is configured for insertion into the console such that the pumps interface with purging-fluid tube 196a and/or waste tube 196b so as to pump the purging fluid.

In some embodiments, the one or more pumps comprise respective barrels 390, which are connected to the purging-fluid tube and/or the waste tube, and respective plungers or pistons 392 configured to reciprocate within barrels 390, when driven by the motors, so as to pump the purging fluid. In some embodiments, each plunger or piston 392 is driven via a rack and pinion 393. Typically, one-way valves 396 regulate the flow to and from barrels 390. In some embodiments, instead of a single port connected to a T-junction as shown in FIG. 15A, each barrel comprises both an end port and a side port, and one-way valves 396 regulate the flow such that the fluid flows into the barrel through one of the ports and from the barrel through the other port.

In some embodiments, cartridge 214 comprises an electrical interface 397 configured to receive electrical power, typically from the console (i.e., via a corresponding electrical interface 398 belonging to the console). In some such embodiments, a cable 212, which is configured to connect to pressure sensor 206 (via an electrical interface of the pressure sensor) so as to deliver electrical power to the sensor, is connected to electrical interface 397 and exits the cartridge via third port 386c, which, it will be recalled, corresponds to third group 128c. Alternatively or additionally, any internal electrical components of cartridge 214, such as any internal motors or pressure sensors, are powered via interface 397.

In some embodiments, the console comprises a pressure sensor 408, comprising a load cell for example, and cartridge 214 is configured for insertion into the console such that pressure sensor 408 senses the pressure in purging-fluid tube 196a. For example, in some embodiments, the purging-fluid tube comprises an expandable portion 412 within the cartridge, and pressure sensor 408 is configured to sense the pressure by sensing the expansion of the expandable portion, e.g., via a sensor interface 410. In response to the pressure, processor 25 controls the pumping of the purging fluid.

Typically, the console further comprises a latch 413. Latch 413 is configured to close on the cartridge upon the insertion of the cartridge, such that the expansion of the expandable portion (against sensor interface 410) does not cause the cartridge to exit the console.

Typically, console 21 comprises a cable interface 414. Cable 224 connects the console to the intracorporeal device (e.g., to motor unit 23 of the device, shown in FIG. 1A) via cable interface 414.

In some cases, there may be a risk of one or more cables and/or tubes becoming disconnected while the device is in use, particularly if the console is removed from cart 192. For example, in some embodiments, cart 192 comprises a handle 416. If any of the cables or tubes were mistakenly passed through handle 416, the handle might tug on the cable or tube while the console is moved, causing the cable or tube to become disconnected.

To address this risk, in some embodiments, cart 192 is shaped to define a first groove 418a, configured to hold cable 224, a second groove 418b, configured to hold the respective distal portions of purging-fluid tube 196a, waste tube 196b, and pressure-sensing tube 196d, and a third groove 418c, configured to hold the distal portion of flushing tube 196c, the proximal portion of pressure-sensing tube 196d, and, optionally, cable 212. The placement of the tubes and cables in the grooves reduces the risk of disconnection. Typically, to facilitate using the grooves, cart 192 is configured to carry console 21 such that, following the insertion of the cartridge, first groove 418a is aligned with cable interface 414, second groove 418b is aligned with second port 386b, and third groove 418c is aligned with third port 386c.

Alternatively or additionally, there is a gap in handle 416, such that any of the cables or tubes that were accidentally passed through handle 416 can be removed from the handle, via the gap, without disconnecting.

Reference is now made to FIG. 15B, which is a schematic layout of cartridge 214 and associated components, in accordance with some embodiments.

In some embodiments, instead of comprising plungers or pistons, the one or more pumps are peristaltic pumps comprising respective rotors 389 configured to squeeze the purging-fluid tube and/or the waste tube, when driven by the motors, so as to pump the purging fluid. In some embodiments, cartridge 214 comprises rotors 389, and the rotors are connected to motors in the console via one or more mechanical interfaces. In other embodiments, console 21 comprises rotors 389. Cartridge 214 is shaped to define one or more openings 391, and the cartridge is configured for insertion into the console such that during the insertion, the rotors pass through openings 391, respectively, such that the rotors are positioned to squeeze the purging-fluid tube and/or the waste tube.

Typically, following the opening of packaging 162 (FIG. 13), one or more preparatory steps are performed prior to the crimping of the pump-head portion of device 20.

In some embodiments, the preparatory steps include a priming of the purging system, in which some purging fluid is passed through the device, typically after connecting purging-fluid tube 196a and waste tube 196b to purging-fluid bag 198 and waste bag 200, respectively. (Typically, the device is connected to purging-fluid tube 196a and waste tube 196b prior to the packaging of the device. Otherwise, the device is connected to these tubes prior to starting the flow of the purging fluid.) For embodiments comprising cartridge 214, the priming of the purging system is typically performed by inserting the cartridge and then instructing the processor to activate the pump(s).

Typically, some of the purging fluid is required to purge the interface between the axial shaft and any bearings (including radial and/or thrust bearings) at the distal end of the device. Hence, while the purging fluid is passed through the device, the user verifies that some purging fluid exits the distal end of the device, indicating that this interface was purged. Alternatively or additionally, for embodiments in which the purging fluid inflates inflatable element 316, which is shown in FIGS. 11A-11B for example, the user verifies that the inflatable element is inflated. Alternatively or additionally, by checking the output of pressure sensor 408 (which is typically displayed on display 228), the user verifies that the pressure within the purging system is within a predefined range.

Alternatively or additionally, the preparatory steps include a priming of the aortic pressure-sensing system. Typically, this priming is preceded by a calibration of pressure sensor 206, which is typically included in packaging 162. Following the calibration, flushing tube 196c and pressure-sensing tube 196d are connected to the pressure sensor, and flushing tube 196c is connected to flushing-fluid bag 202. (Typically, the device is connected to pressure-sensing tube 196d prior to the packaging of the device. Otherwise, this connection is made before starting the priming of the aortic pressure-sensing system.) Subsequently, flushing tube 196c and pressure-sensing tube 196d are flushed with the flushing fluid, typically at a flow rate that is higher than the usual flow rate of the flushing fluid while the device is in use within the body of the subject. (The usual flow rate is relatively low, such that the flushing does not interfere with the pressure sensing.) For example, in some embodiments, the usual flow rate is less than 4 mL/hr (e.g., the usual flow rate is 3 mL/hr), and the higher flow rate is at least 4 mL/hr. In some embodiments, to facilitate the higher flow rate of the flushing fluid, a valve 210 of pressure sensor 206 is opened during the priming.

Typically, the flushing fluid also flows through aortic pressure-sensing channel 147 (FIG. 5B), which is in fluid communication with pressure-sensing port 179. In some embodiments, an additional flushing of the aortic pressure-sensing channel, at an even higher flow rate, is performed. For example, in some embodiments, flushing fluid is expelled rapidly, e.g., from a syringe, into pressure-sensing port 179, e.g., via stopcock 384.

Typically, prior to flushing the pressure-sensing channel (e.g., prior to performing any priming of the pressure-sensing system), chamber 164 (FIGS. 12A-12C) is at least partly filled with a liquid, such as saline. In addition to help preventing air from entering the delivery catheter during the crimping of pump-head portion 27 (FIGS. 12A-12C), the liquid allows the user to see any bubbles escaping from the pressure-sensing channel. Upon the user verifying that no more bubbles are escaping, the flow of flushing fluid is stopped.

For some applications, processor 25 guides a user through a sequence of one or more of the preparatory steps by providing instructions via a user interface of console 21, such as display 228. For some applications, the processor automatically performs a sequence of one or more of the preparatory steps by receiving instructions to do so via a user interface of console 21, such as display 228.

Following the preparatory steps, pump-head portion 27 is crimped, e.g., as described above with reference to FIGS. 12A-12C. For embodiments in which the purging fluid inflates inflatable element 316, which is shown in FIGS. 9A-9B for example, the inflatable element is deflated prior to the crimping. For example, in some embodiments, pump 388b is activated without activating pump 388a, such that pump 388b suctions all the purging fluid (except for any purging fluid between purging-fluid bag 198 and pump 388a) from the purging system. Subsequently, during the crimping of pump-head portion 27, the purging system is inactive, so as not to reinflate the inflatable element. Following the crimping, the purging system is reactivated. Thus, after the device has been inserted into the body of the subject, the purging fluid continues to purge the interface between the axial shaft and the bearings. In the event a repositioning is required or the device is to be withdrawn, the purging fluid is suctioned from the device, such that inflatable element 316 is deflated, and pump-head portion 27 is then crimped.

More generally, the scope of the present disclosure includes passing purging fluid through any intracorporeal device, either before or after insertion of the intracorporeal device into the body of a subject, so as to inflate an inflatable element of the intracorporeal device with the fluid and also to purge an interface between a first component of the intracorporeal device and a second component of the intracorporeal device. The scope of the present disclosure further includes deflating the inflatable element by suctioning the purging fluid from the intracorporeal device, and subsequently to deflating the inflatable element, crimping a self-expandable element of the intracorporeal device.

Reference is now additionally made to FIG. 13. In some embodiments, tray 163 comprises a compartment 165 configured to hold at least one air-eliminating filter in an upright position while the purging fluid flows (e.g., while the purging fluid is pumped by pump 388a) from purging-fluid bag 198, via purging-fluid tube 196a and the air-eliminating filter, into inlet port 86.

Typically, the upright position is required only while the channels of the air-eliminating filter are initially filled, e.g., during the priming of the purging system. Subsequently, e.g., during operation of device 20, the upright position—and hence, compartment 165—is not required. Typically, proximal air-eliminating filter 378a is hung in an upright position from cart adjunct 204, such that compartment 165 is required only for distal air-eliminating filter 378b.

It is noted that tray 163 can comprise compartment 165 even without one or more of the other features of packaging 162 described above with reference to FIGS. 12A-12C and FIG. 13.

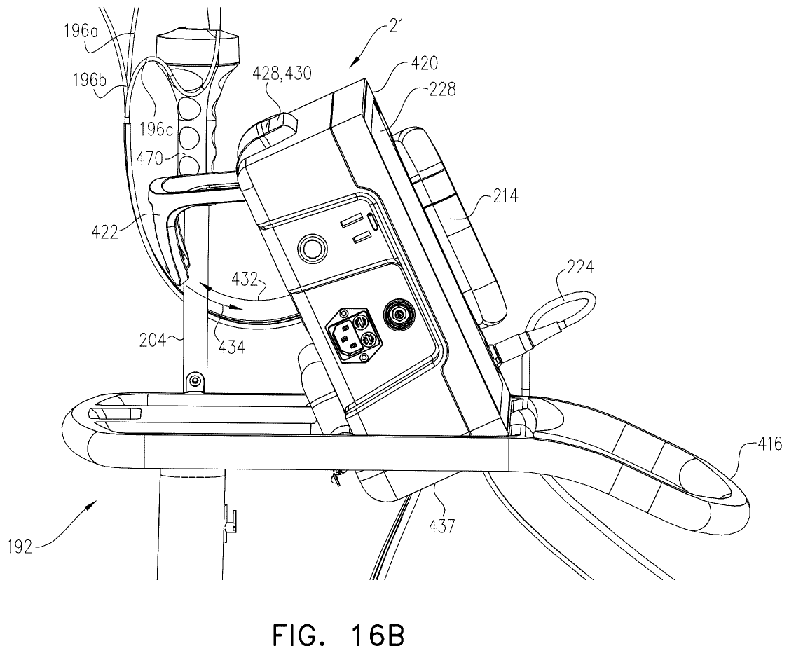

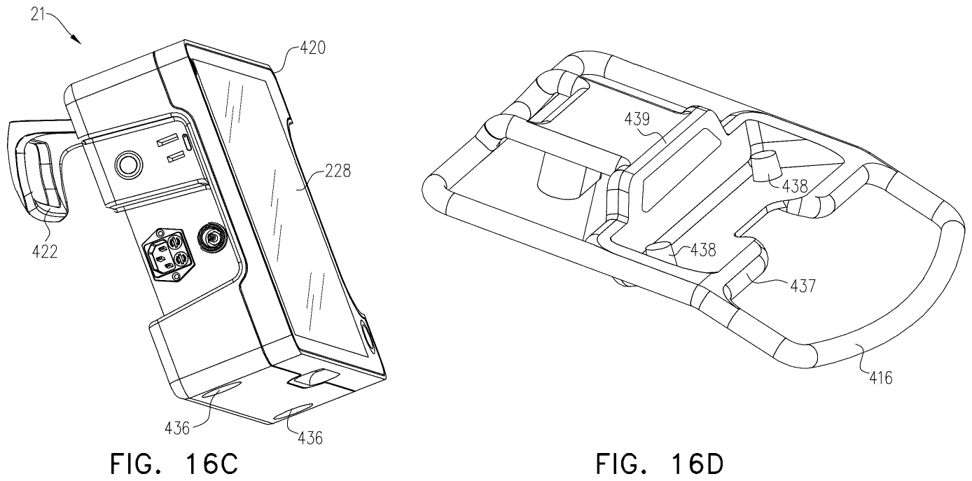

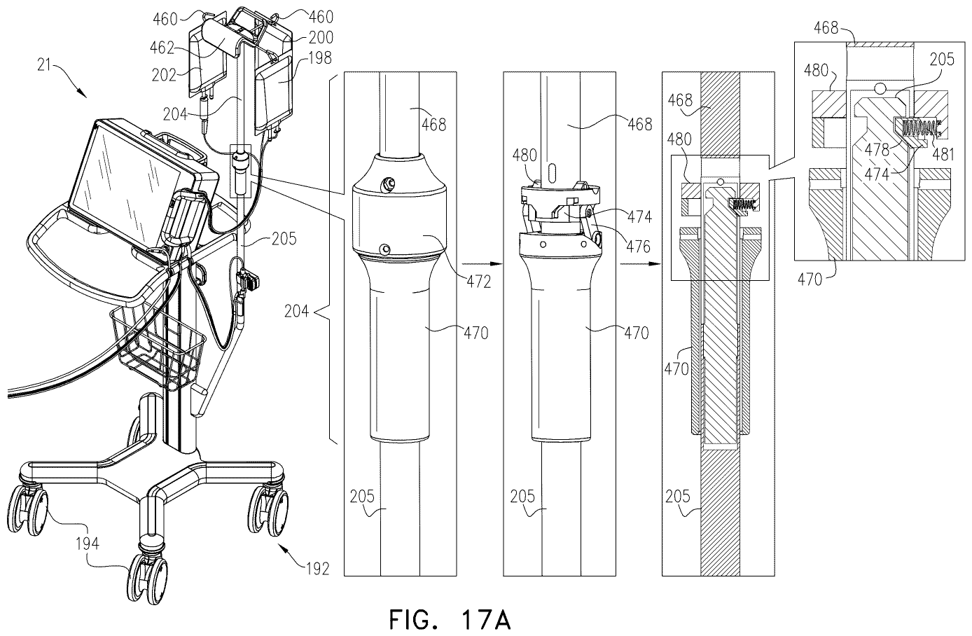

Reference is now made to FIG. 16A, which is a schematic illustration of console 21 as viewed from behind, and to FIG. 16B, which is a schematic illustration of the console as viewed from the side, in accordance with some embodiments.

In some embodiments, console 21 comprises a hook 422 coupled to chassis 420. The console is removable from cart 192 and is hangable, via hook 422, from a bedrail of the subject's bed, as shown in FIG. 17B, which is described below. Typically, for embodiments in which the console comprises display 228, the display is at the front of the chassis, while hook 422 is at the rear of the chassis.

Typically, hook 422 is rotatably coupled to chassis 420 such that the hook is rotatable from a closed position, in which the hook does not protrude from the chassis, to an open position, in which the hook protrudes from the chassis, as indicated by a rotation indicator 432 in FIG. 16B. In some embodiments, as shown in FIG. 16A, the chassis is shaped to define a groove 424, and hook 422 fits into the groove in the closed position.

Typically, chassis 420 comprises a ratchet 426 and a release mechanism 428 configured to release ratchet 426. (Ratchet 426 is typically an internal component of the chassis, and is thus hidden from view in FIGS. 16A-16B.) Hook 422 is coupled to the chassis via ratchet 426 such that an activation of release mechanism 428 (i.e., a releasing of the ratchet) causes the hook to rotate from the closed position to the open position. Following the rotation to the open position, the user rotates the hook, as indicated by another rotation indicator 434 in FIG. 16B, from the open position to a partially-closed position in which the hook secures the console on the bedrail. Following the rotation of the hook to the partially-closed position, ratchet 426 maintains the hook in the partially-closed position.

In some embodiments, release mechanism 428 comprises a handle 430 coupled to ratchet 426, and the activation of the release mechanism includes lifting handle 430. Thus, when the user lifts handle 430 so as to carry the console, via the handle, from the cart to the bedrail, hook 422 rotates to the open position. In other words, no separate ratchet-release operation is required, but rather, the user may simply lift the console via the handle.

Reference is now made to FIG. 16C, which is a schematic illustration of console 21, and to FIG. 16D, which is a schematic illustration of a portion of cart 192, in accordance with some embodiments.

Typically, chassis 420 is shaped to define one or more indentations 436, and cart 192 comprises one or more protrusions 438 configured to fit into the indentations while the cart carries the console, such that the console does not fall from the cart. For example, in some embodiments, protrusions 438 protrude upward from tray 437, which is configured to carry the console. In some such embodiments, the cart further comprises a backstop 439, which is behind the tray, the tray slants downward toward backstop 439, and protrusions 438 are spaced from the backstop so as to facilitate a backward tilt of the console while the console is on the tray. This backward tilt facilitates use of the console, particularly display 228 of the console.

Alternatively or additionally to the fitting of protrusions 438 into indentations 436, the console is magnetically held in place on the cart. For example, in some embodiments, protrusions 438 are at least partly magnetic, and chassis 420 comprises respective ferromagnetic elements adjacent to indentations 436. Alternatively, protrusions 438 are at least partly ferromagnetic, and chassis 420 comprises respective magnetic elements adjacent to indentations 436. Alternatively or additionally, the console is lockable in place via a mechanical lock.

Reference is now made to FIG. 17A, which schematically shows several enlarged views of a portion of cart adjunct 204 and post 205, in accordance with some embodiments. Reference is also made to FIG. 17B, which is a schematic illustration of cart adjunct 204 and console 21 hanging from a bedrail 464 of the subject's bed 466, in accordance with some embodiments. (For ease of illustration, the tubes connected to the fluid bags are omitted from FIG. 17B.)

Cart adjunct 204 comprises one or more bag-holding appendages 460, which in some embodiments comprise hooks or clips, configured to hold the fluid bags. Cart adjunct 204 further comprises a coupling element 462, such as a hook or a clip (e.g., a carabiner). As described above with reference to FIG. 14, typically, cart adjunct 204 is removably couplable to cart 192. Following the uncoupling of the cart adjunct from the cart, the cart adjunct can be coupled, via coupling element 462, to bedrail 464, e.g., the cart adjunct can be hung from the bedrail. As described above with reference to FIGS. 16A-16B, console 21 is also removable from the cart. Thus, advantageously, even while the device is in use, bed 466 can be moved independently from the cart.

Typically, as illustrated in the leftmost enlarged view of FIG. 17A, cart adjunct 204 comprises a pole 468, the top portion of which is coupled to bag-holding appendages 460 and to coupling element 462. In some embodiments, the cart adjunct is coupled to the cart by inserting post 205 into pole 468, such that the bottom portion of the pole is disposed over the top portion of the post. In other embodiments, pole 468 is inserted into post 205.

In some embodiments, cart adjunct 204 further comprises a slider 470, the function of which is described below. Typically, for embodiments in which pole 468 is disposed over post 205, slider 470 is at the bottom of the cart adjunct.

In some embodiments, cart adjunct 204 further comprises a non-functional cap 472, which is hidden from view in the middle enlarged view of FIG. 17A so as to expose additional components of cart adjunct 204, the functions of which are described below. The rightmost enlarged view of FIG. 17A shows a longitudinal cross section through the middle enlarged view of FIG. 17A.

In some embodiments, post 205 is shaped to define a groove 478. Cart adjunct 204 comprises a latch 474 and a spring 481, which is configured to lock the cart adjunct to the post by pushing latch 474 into groove 485. Cart adjunct 204 further comprises a latch release configured to unlock the cart adjunct from post 205 by releasing latch 474 from groove 485. To decouple the cart adjunct from the cart, the user activates the latch release and lifts the cart adjunct.

In some embodiments, the latch release comprises slider 470. Slider 470 is coupled to latch 474 and is configured to release the latch by sliding over post 205. Typically, the slider is configured to release the latch by sliding upward, such that an upward sliding of the slider releases the latch and also lifts the cart adjunct from the cart. Thus, advantageously, the user can release the latch and lift the cart adjunct with a single lifting action.

For example, in some embodiments, slider 470 is coupled to latch 474 via one or more arms 476. As slider 470 slides vertically toward the latch, arms 476 rotate, thereby horizontally sliding the latch out of groove 478. In some embodiments, pole 468 is coupled to a latch frame 480, which defines a track in which latch 474 slides.

It is noted that the scope of the present disclosure includes the use of the latch and slider mechanism, as described above, to couple any cart adjunct to any cart, regardless of the functions of these two elements.

Reference is now made to FIG. 18A, which is a schematic illustration of a guide 446 for facilitating repositioning an intracorporeal device, which includes a valve 448 at the proximal end of the device, within a body of a subject, in accordance with some embodiments. As a particular example of an intracorporeal device, FIG. 18A shows the proximal end of ventricular assist device 20, which comprises driven-magnet unit 310. Reference is also made to FIG. 18B, which shows a schematic longitudinal cross-section through guide 446 and driven-magnet unit 310, in accordance with some embodiments.

In some cases, an intracorporeal device requires repositioning within the body of the subject. For example, in the case of ventricular assist device 20, the distal-tip portion of the device, which, it will be recalled, is typically supposed to be positioned at the apex of the left ventricle, may be incorrectly positioned. Alternatively, the pump-head portion of the device may migrate from the left ventricle into the aorta. In such cases, for safety, it is typically preferable to radially constrain the pump-head portion within the delivery catheter and then reposition the device over guidewire 10 (FIG. 1B). However, to reinsert the guidewire without completely withdrawing the device from the body of the subject, the distal end of the guidewire must be inserted through the proximal end of the device. This insertion can be challenging due to the presence of valve 448, and because, typically, the distal end of the guidewire is soft and atraumatic. (In contrast, prior to inserting the device into the body, the proximal end of the guidewire, which is typically firmer than the distal end of the guidewire, is inserted via the distal end of the device.)

To address this challenge, guide 446 facilitates insertion of the distal end of the guidewire through valve 448. Guide 446 comprises a tube 450, which is configured to radially constrain the guidewire by virtue of the inner diameter of tube 450 being only slightly greater than the diameter of the guidewire. Guide 446 further comprises a tube shell 452, which contains tube 450. Tube shell 452 is shaped to define a proximal shell opening 454, which is in communication with the proximal end of tube 450. For example, opening 454 may open directly into the proximal end of tube 450, or may open into a lumen of the tube shell that opens into tube 450. Tube shell 452 comprises a hollow distal shell portion 456, which contains the distal end of tube 450.

To reposition the intracorporeal device, the intracorporeal device is partially withdrawn from the subject's body. For example, in some embodiments, the distal end of ventricular assist device 20 is withdrawn to the descending aorta. Subsequently, distal shell portion 456 is placed over the proximal end of the device, as indicated by a placement indicator 458, until the distal end of tube 450 passes through valve 448. Subsequently, the distal end of the guidewire is inserted into the proximal end of the intracorporeal device via proximal shell opening 454 and tube 450.

Typically, for ventricular assist device 20, prior to placing distal shell portion 456 over the proximal end of the device (and, optionally, prior to partially withdrawing the device), the proximal end of the device is decoupled from motor unit 23 (FIG. 1).

Typically, the inner diameter of tube 450 is between 0.4 and 0.8 mm, such as between 0.5 and 0.7 mm. As a specific example, in some embodiments, the inner diameter of the tube is approximately 0.6 mm, which is slightly greater than the diameter of a 0.018 inch guidewire.

Typically, to facilitate the insertion of the guidewire, proximal shell opening 454 is conical.

In some embodiments, for ease of manufacture, tube shell 452 comprises two parts, which are coupled together: a proximal part 452p, which is shaped to define opening 454, and a distal part 452d, which comprises hollow distal shell portion 456.

In some embodiments, distal shell portion 456 comprises an O-ring 482. O-ring 482 has an inner diameter that is slightly smaller than the diameter of the proximal end of the device, such that the O-ring, by fitting snugly around the proximal end of the device, stabilizes and centers tube 450 with respect to valve 448.

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description.

1. An apparatus for use with a purging-fluid bag containing purging fluid and an intracorporeal device including an inlet port, the apparatus comprising:
a proximal air-eliminating filter, configured to remove air from the purging fluid;
a distal air-eliminating filter, configured to remove air from the purging fluid; and
a purging-fluid tube, configured to connect to the purging-fluid bag via the proximal air-eliminating filter, and to connect to the inlet port via the distal air-eliminating filter, such that the purging fluid flows from the purging-fluid bag, via the proximal air-eliminating filter, purging-fluid tube, and distal air-eliminating filter, into the inlet port.

2. The apparatus according to claim 1, wherein the proximal air-eliminating filter comprises a proximal air-filtering membrane shaped to define proximal pores, and wherein the distal air-eliminating filter comprises a distal air-filtering membrane shaped to define distal pores, which are smaller than the proximal pores.

3. The apparatus according to claim 1,
wherein the intracorporeal device further includes a self-expandable element,
wherein the apparatus further comprises packaging comprising a tray shaped to define a chamber in which the self-expandable element is packageable in a non-radially-constrained configuration, and
wherein the tray comprises a compartment configured to hold the distal air-eliminating filter in an upright position while the purging fluid flows from the purging-fluid bag, via the purging-fluid tube and distal air-eliminating filter, into the inlet port.

4. The apparatus according to claim 3,
wherein the intracorporeal device further includes a delivery catheter coupled to the inlet port and an elongate element passing through the delivery catheter,
wherein the self-expandable element is coupled to the elongate element distally to the elongate element and is configured for percutaneous delivery to a portion of a body of a subject while the self-expandable element is in a radially-constrained configuration within the delivery catheter, and
wherein the packaging further comprises a securement piece coupled to the tray adjacently to the chamber, and configured to secure a distal end of the delivery catheter while the self-expandable element is retracted into the delivery catheter via retraction of the elongate element.

5. The apparatus according to claim 4, wherein a portion of the tray underneath the securement piece slopes downwardly in a direction of the chamber.

6. The apparatus according to claim 4,
wherein the device further includes a proximal element, which is disposed proximally to the delivery catheter and is wider than the delivery catheter,
wherein the elongate element passes through the proximal element, and
wherein the tray is configured to stabilize the proximal element while the self-expandable element is retracted into the delivery catheter.

7. The apparatus according to claim 6, wherein the proximal element includes a fixation unit configured to fix a position of the elongate element relative to the delivery catheter.

8. The apparatus according to claim 6,
wherein the tray is further shaped to define a track in which the delivery catheter is packageable, and
wherein the packaging further comprises a detachable element reversibly coupled to the tray over the track and configured to stabilize the proximal element when the proximal element is pushed against the detachable element.

9. The apparatus according to claim 6,
wherein the tray is further shaped to define a track in which the delivery catheter is packageable, and
wherein the track comprises a widened portion configured to stabilize the proximal element when the proximal element is pushed against a wall of the widened portion.

10. A method, comprising:
connecting a proximal end of a purging-fluid tube to a purging-fluid bag, which contains purging fluid, via a proximal air-eliminating filter configured to remove air from the purging fluid; and
connecting a distal end of the purging-fluid tube to an inlet port of an intracorporeal device via a distal air-eliminating filter configured to remove air from the purging fluid, such that the purging fluid flows from the purging-fluid bag, via the proximal air-eliminating filter, purging-fluid tube, and distal air-eliminating filter, into the inlet port.

11. The method according to claim 10, wherein the proximal air-eliminating filter includes a proximal air-filtering membrane shaped to define proximal pores, and wherein the distal air-eliminating filter includes a distal air-filtering membrane shaped to define distal pores, which are smaller than the proximal pores.

12. The method according to claim 10,
wherein the device includes:
a delivery catheter coupled to the inlet port,
an elongate element passing through the delivery catheter, and
a self-expandable element coupled to the elongate element distally to the elongate element and configured for percutaneous delivery to a portion of a body of a subject while the self-expandable element is in a radially-constrained configuration within the delivery catheter, and
wherein the method further comprises, subsequently to connecting the purging-fluid tube to the purging-fluid bag and to the inlet port, retracting the self-expandable element into the delivery catheter by retracting the elongate element.

13. The method according to claim 12,
wherein the device further includes a first component and a second component, and
wherein the purging fluid purges an interface between the first component and the second component by flowing through the device via the inlet port.

14. The method according to claim 13,
wherein the device further includes an inflatable element, and
wherein the purging fluid inflates the inflatable element by flowing through the device via the inlet port.

15. The method according to claim 14, further comprising, prior to retracting the elongate element, deflating the inflatable element by suctioning the purging fluid from the device.

16. The method according to claim 14, wherein the inflatable element surrounds the elongate element.

17. The method according to claim 16, wherein a wall of the elongate element is shaped to define one or more inflation-fluid openings, wherein the inflatable element surrounds the inflation-fluid openings, and wherein the purging fluid inflates the inflatable element via the inflation-fluid openings.

18. The method according to claim 13,
wherein the self-expandable element includes:
a pump-outlet tube, which is configured for insertion, through an aorta of the subject, into a left ventricle of a heart of the subject such that a proximal portion of the pump-outlet tube is disposed within the aorta and a distal portion of the pump-outlet tube is disposed within the left ventricle, and
an impeller configured to pump blood of the subject proximally through the pump-outlet tube, such that the blood exits the proximal portion of the pump-outlet tube,
wherein the first component of the intracorporeal device includes an axial shaft coupled to the impeller and configured to rotate such that the impeller pumps the blood, and
wherein the second component of the intracorporeal device includes at least one bearing configured not to rotate with the axial shaft.

19. The method according to claim 12,
wherein the device defines a pressure-sensing channel, and
wherein the method further comprises, prior to retracting the elongate element, flushing the pressure-sensing channel, with a flushing fluid, at a flow rate higher than a usual flow rate at which the flushing fluid flows through the pressure-sensing channel while the device is in use within the body of the subject.

20. The method according to claim 19, wherein the pressure-sensing channel is between the delivery catheter and the elongate element.

21. The method according to claim 19,
wherein the device is packaged in a tray shaped to define a chamber, the self-expandable element being packaged in the chamber, and
wherein the method further comprises, prior to flushing the pressure-sensing channel, at least partly filling the chamber with a liquid.

22. The method according to claim 12,
wherein the device is packaged in a tray,
wherein the device further includes a proximal element, which is disposed proximally to the delivery catheter and is wider than the delivery catheter, and
wherein retracting the elongate element comprises retracting the elongate element while stabilizing the proximal element using the tray.

23. The method according to claim 22, wherein the tray is shaped to define a track in which the delivery catheter is packaged, wherein the track includes a widened portion, and wherein stabilizing the proximal element comprises stabilizing the proximal element by pushing the proximal element against a wall of the widened portion.
